SEQUENCE LISTING (96 sequences)

SEQ ID NO: 1 - RNA, 24 nt, Artificial sequence
  Synthetic oligo
aacccaggaggcggaacuugcagu

SEQ ID NO: 2 - RNA, 20 nt, Artificial sequence
  Synthetic oligo
aauagccacugcacuccagc

SEQ ID NO: 3 - RNA, 19 nt, Artificial sequence
  Synthetic oligo
acccaggaggcagagguug

SEQ ID NO: 4 - RNA, 25 nt, Artificial sequence
  Synthetic oligo
acugcacuccagccugggcaacaua

SEQ ID NO: 5 - RNA, 18 nt, Artificial sequence
  Synthetic oligo
aggcuggagugcaauggc

SEQ ID NO: 6 - RNA, 22 nt, Artificial sequence
  Synthetic oligo
agguggaucaccugaggucagg

SEQ ID NO: 7 - RNA, 17 nt, Artificial sequence
  Synthetic oligo
aguacuuggaugggaga

SEQ ID NO: 8 - RNA, 22 nt, Artificial sequence
  Synthetic oligo
aguucgagaccagccugggcaa

SEQ ID NO: 9 - RNA, 21 nt, Artificial sequence
  Synthetic oligo
cacugcacuccagccugggca

SEQ ID NO: 10 - RNA, 22 nt, Artificial sequence
  Synthetic oligo
cacugcacucuagccugggcga

SEQ ID NO: 11 - RNA, 18 nt, Artificial sequence
  Synthetic oligo
caggcuggucucgaacuc

SEQ ID NO: 12 - RNA, 20 nt, Artificial sequence
  Synthetic oligo
cugggcaacauagcgagacc

SEQ ID NO: 13 - RNA, 20 nt, Artificial sequence
  Synthetic oligo
cugggcaacauagcgagacu

SEQ ID NO: 14 - RNA, 18 nt, Artificial sequence
  Synthetic oligo
gcaguggcgcgaucucgg

SEQ ID NO: 15 - RNA, 18 nt, Artificial sequence
  Synthetic oligo
gccgagaucgcgccacug

SEQ ID NO: 16 - RNA, 23 nt, Artificial sequence
  Synthetic oligo
ggaggaucgcuugaguccaggag

SEQ ID NO: 17 - RNA, 22 nt, Artificial sequence
  Synthetic oligo
ggggauguagcucagugguaga

SEQ ID NO: 18 - RNA, 24 nt, Artificial sequence
  Synthetic oligo
ggggguguagcucagugguagagc

SEQ ID NO: 19 - RNA, 16 nt, Artificial sequence
  Synthetic oligo
gucccggcggagucgc

SEQ ID NO: 20 - RNA, 18 nt, Artificial sequence
  Synthetic oligo
guugcccaggcuggagug

SEQ ID NO: 21 - RNA, 23 nt, Artificial sequence
  Synthetic oligo
uacuugaccuugacucucccuca

SEQ ID NO: 22 - RNA, 22 nt, Artificial sequence
  Synthetic oligo
ugaccucgugaucugcccgccu

SEQ ID NO: 23 - RNA, 25 nt, Artificial sequence
  Synthetic oligo
uuuacugacacuguucuuuuucuag

SEQ ID NO: 24 - RNA, 23 nt, Artificial sequence
  Synthetic oligo
uuuaguagagacgggguuucacc

SEQ ID NO: 25 - RNA, 22 nt, Artificial sequence
  Synthetic oligo
uuuuguauuuuuaguagagaca

SEQ ID NO: 26 - RNA, 22 nt, Artificial sequence
  Synthetic oligo
aauuucugcauaguccaccucu

SEQ ID NO: 27 - RNA, 23 nt, Artificial sequence
  Synthetic oligo
acugcaaccucugccucccgggu

SEQ ID NO: 28 - RNA, 18 nt, Artificial sequence
  Synthetic oligo
agacggagucucgcucug

SEQ ID NO: 29 - RNA, 23 nt, Artificial sequence
  Synthetic oligo
agagucuugcucuguugccuagg

SEQ ID NO: 30 - RNA, 26 nt, Artificial sequence
  Synthetic oligo
aggcaggagaaucacuugaacccggg

SEQ ID NO: 31 - RNA, 22 nt, Artificial sequence
  Synthetic oligo
aggcggaucaccugaggucagg

SEQ ID NO: 32 - RNA, 28 nt, Artificial sequence
  Synthetic oligo
aggcugaggcaggagaaucacuugaacc

SEQ ID NO: 33 - RNA, 18 nt, Artificial sequence
  Synthetic oligo
aggcuggagugcacuggc

SEQ ID NO: 34 - RNA, 22 nt, Artificial sequence
  Synthetic oligo
aguucgagaccagccugaccaa

SEQ ID NO: 35 - RNA, 21 nt, Artificial sequence
  Synthetic oligo
caacauagcaagacuccaucu

SEQ ID NO: 36 - RNA, 20 nt, Artificial sequence
  Synthetic oligo
cacccaggcuggagagcagu

SEQ ID NO: 37 - RNA, 25 nt, Artificial sequence
  Synthetic oligo
cacugcaaccuccgccucccagguu

SEQ ID NO: 38 - RNA, 21 nt, Artificial sequence
  Synthetic oligo
cacugcacuccagccugggcg

SEQ ID NO: 39 - RNA, 23 nt, Artificial sequence
  Synthetic oligo
cagccugggcaacagagcgagac

SEQ ID NO: 40 - RNA, 20 nt, Artificial sequence
  Synthetic oligo
cagggucucgcucugucgcc

SEQ ID NO: 41 - RNA, 25 nt, Artificial sequence
  Synthetic oligo
caugugucuuuauaguagaaugauu

SEQ ID NO: 42 - RNA, 21 nt, Artificial sequence
  Synthetic oligo
ccacugcacuccagccugggc

SEQ ID NO: 43 - RNA, 18 nt, Artificial sequence
  Synthetic oligo
ccaggcuggagugcagug

SEQ ID NO: 44 - RNA, 20 nt, Artificial sequence
  Synthetic oligo
cccagcuacucgggaggcug

SEQ ID NO: 45 - RNA, 23 nt, Artificial sequence
  Synthetic oligo
cccgggaggcggagcuugcagug

SEQ ID NO: 46 - RNA, 23 nt, Artificial sequence
  Synthetic oligo
cgggcacaguggcucacgccugu

SEQ ID NO: 47 - RNA, 23 nt, Artificial sequence
  Synthetic oligo
cucacgccuguaaucccagcacc

SEQ ID NO: 48 - RNA, 19 nt, Artificial sequence
  Synthetic oligo
cucacugcaaccucugccu

SEQ ID NO: 49 - RNA, 23 nt, Artificial sequence
  Synthetic oligo
cugaagaucuaaaggucccuggu

SEQ ID NO: 50 - RNA, 24 nt, Artificial sequence
  Synthetic oligo
cugaagaucuaaaggucccugguu

SEQ ID NO: 51 - RNA, 18 nt, Artificial sequence
  Synthetic oligo
cugaccucgugauccgcc

SEQ ID NO: 52 - RNA, 23 nt, Artificial sequence
  Synthetic oligo
gaggacuguauuugugacuaauu

SEQ ID NO: 53 - RNA, 25 nt, Artificial sequence
  Synthetic oligo
gagguguuuauaguauucucugaug

SEQ ID NO: 54 - RNA, 19 nt, Artificial sequence
  Synthetic oligo
gagucucgcucugucgccc

SEQ ID NO: 55 - RNA, 24 nt, Artificial sequence
  Synthetic oligo
gccaagaucgcgccacugcacucc

SEQ ID NO: 56 - RNA, 19 nt, Artificial sequence
  Synthetic oligo
gcgcgcggcggcggcggcg

SEQ ID NO: 57 - RNA, 18 nt, Artificial sequence
  Synthetic oligo
gcucacgccuguaauccc

SEQ ID NO: 58 - RNA, 26 nt, Artificial sequence
  Synthetic oligo
gcugaggcaggagaaucgcuugaacc

SEQ ID NO: 59 - RNA, 22 nt, Artificial sequence
  Synthetic oligo
ggggguauagcucagugguaga

SEQ ID NO: 60 - RNA, 20 nt, Artificial sequence
  Synthetic oligo
gguuaguacuuggaugggag

SEQ ID NO: 61 - RNA, 18 nt, Artificial sequence
  Synthetic oligo
gucgagaucgcgccacug

SEQ ID NO: 62 - RNA, 23 nt, Artificial sequence
  Synthetic oligo
uaauuuuuuguauuuuuaguaga

SEQ ID NO: 63 - RNA, 18 nt, Artificial sequence
  Synthetic oligo
ucacugcaaccuccgccu

SEQ ID NO: 64 - RNA, 24 nt, Artificial sequence
  Synthetic oligo
ucacugcaagcuccgccucccggg

SEQ ID NO: 65 - RNA, 25 nt, Artificial sequence
  Synthetic oligo
ugaggcaggagaaucgcuugaaccu

SEQ ID NO: 66 - RNA, 22 nt, Artificial sequence
  Synthetic oligo
uugaaagagaggaaaagaagcu

SEQ ID NO: 67 - RNA, 22 nt, Artificial sequence
  Synthetic oligo
uuggccaggcuggucucgaacu

SEQ ID NO: 68 - RNA, 21 nt, Artificial sequence
  Synthetic oligo
aauuuuuguauuuuugguaga

SEQ ID NO: 69 - RNA, 20 nt, Artificial sequence
  Synthetic oligo
acccaggcuggagugcagug

SEQ ID NO: 70 - RNA, 23 nt, Artificial sequence
  Synthetic oligo
aggcaggagaauugcuugaaccc

SEQ ID NO: 71 - RNA, 22 nt, Artificial sequence
  Synthetic oligo
agugcaguggcgcgaucucggc

SEQ ID NO: 72 - RNA, 18 nt, Artificial sequence
  Synthetic oligo
cccaggcuggaguacagu

SEQ ID NO: 73 - RNA, 21 nt, Artificial sequence
  Synthetic oligo
cgacauagcaagacuccaucu

SEQ ID NO: 74 - RNA, 21 nt, Artificial sequence
  Synthetic oligo
cgcccaggcuggagugcagug

SEQ ID NO: 75 - RNA, 21 nt, Artificial sequence
  Synthetic oligo
cgccccaggucucggucccug

SEQ ID NO: 76 - RNA, 26 nt, Artificial sequence
  Synthetic oligo
cugaggugggaggaucgcuugagccu

SEQ ID NO: 77 - RNA, 18 nt, Artificial sequence
  Synthetic oligo
cugggacuacaggcaccc

SEQ ID NO: 78 - RNA, 18 nt, Artificial sequence
  Synthetic oligo
gagucuugcucugucgcc

SEQ ID NO: 79 - RNA, 17 nt, Artificial sequence
  Synthetic oligo
gcgcggcggcggcggcg

SEQ ID NO: 80 - RNA, 16 nt, Artificial sequence
  Synthetic oligo
gcuacucgggaggcug

SEQ ID NO: 81 - RNA, 18 nt, Artificial sequence
  Synthetic oligo
gcugagaucacaccacug

SEQ ID NO: 82 - RNA, 20 nt, Artificial sequence
  Synthetic oligo
ggccgggcaugguggcucac

SEQ ID NO: 83 - RNA, 22 nt, Artificial sequence
  Synthetic oligo
ggguguagcucagugguagagc

SEQ ID NO: 84 - RNA, 18 nt, Artificial sequence
  Synthetic oligo
gucucgaacuccugaccu

SEQ ID NO: 85 - RNA, 22 nt, Artificial sequence
  Synthetic oligo
uccugaccucgugauccgcccg

SEQ ID NO: 86 - RNA, 22 nt, Artificial sequence
  Synthetic oligo
ugcugggauuacaggugugagc

SEQ ID NO: 87 - RNA, 27 nt, Artificial sequence
  Synthetic oligo
uguagucccagcuacucgggaggcuga

SEQ ID NO: 88 - RNA, 24 nt, Artificial sequence
  Synthetic oligo
uugauuugcauuucucugauggcc

SEQ ID NO: 89 - RNA, 18 nt, Artificial sequence
  Synthetic oligo
uuuguauuuuuaguagag

SEQ ID NO: 90 - DNA, 26 bp, Artificial sequence
  Synthetic oligo
ttaaccttttggtgtttgatgagtgt

SEQ ID NO: 91 - DNA, 23 bp, Artificial sequence
  Synthetic oligo
ggacatgatggacaattttcaca

SEQ ID NO: 92 - RNA, 22 nt, Artificial sequence
  Synthetic oligo
ggucguaugugugacgccauuu

SEQ ID NO: 93 - RNA, 22 nt, Artificial sequence
  Synthetic oligo
ucgacagcacgacacugccuuc

SEQ ID NO: 94 - RNA, 21 nt, Artificial sequence
  Synthetic oligo
uaauuuuauguauaagcuagu

SEQ ID NO: 95 - RNA, 23 nt, Artificial sequence
  Synthetic oligo
ugauuguagccuuuuggaguaga

SEQ ID NO: 96 - DNA, 21 bp, Artificial sequence
  Synthetic oligo
gtgcagtgctgcatctctggt

(19) United States
(12) Patent Application Publication
(10) Pub. No.: US 2020/0165609 A1
(43) Pub. Date: May 28, 2020

(54) METHODS OF IDENTIFYING MIRNAS AND APPLICATIONS THEREOF

(71) Applicant: THE CHILDREN'S HOSPITAL OF PHILADELPHIA, Philadelphia, PA (US)

(72) Inventors: Dimitri MONOS, Merion, PA (US); Peter CLARK, Philadelphia, PA (US)

(73) Assignee: THE CHILDREN'S HOSPITAL OF PHILADELPHIA, Philadelphia, PA (US)

(21) Appl. No.: 16/611,293

(22) PCT Filed: May 10, 2018
(86) PCT No.: PCT/US2018/031966

§ 371 Date: Nov. 6, 2019

Related U.S. Application Data
(60) Provisional application No. 62/504,267, filed on May 10, 2017.

(51) Int. Cl. C12N 15/113; G16B 15/10; G16B 30/20; C12Q 1/6886

(52) U.S. Cl. CPC C12N 15/113; G16B 15/10; G16B 30/20; C12Q 2600/178; C12N 2310/141; C12N 2320/10; C12Q 1/6886

(57) ABSTRACT

Accurate mapping of RNA-Seq reads to the reference genome is critical for accurate identification and quantification of miRNA transcripts. The miRNA transcriptome of two homozygous lymphoblastoic cell lines with completely characterized MHC haplotypes (PGF and COX) was analyzed and revealed 89 novel mature miRNA transcripts originating from within the MHC, including three mature miRNA that are haplotype specific, one of which originates from within intron 5 of HLA-DRB5. This is a novel way to identify miRNA transcripts originating from the MHC in a variety of tissue types and disease states, and can be utilized to prepare personal genome assemblies.

This application claims benefit of priority to U.S. Provisional Application Ser. No. 62/504,267, filed May 10, 2017, the entire contents of which are hereby incorporated by reference.

### BACKGROUND

### I. Field

The present disclosure relates generally to the fields of genetics, medicine and gene regulation. More particularly, they relate to the identification of miRNA sequences.

### II. Related Art

A. miRNAs

MicroRNAs (miRNAs) are an abundant class of short non-coding RNAs (ncRNAs), −22 nucleotides (nts) in length, which play a significant role in the regulation of gene expression (Bartel, 2004 and Bartel, 2009). The first animal miRNA, lin-4, was discovered accidentally during a genetic screen in Caenorhabditis elegans (C. elegans) where it was previously reported to repress the expression of a protein-coding gene lin-14 (Ambros, 1989; Ruvkun et al., 1989). Down-regulation of lin-14 led to a change in pattern formation in C. elegans larvae. In 2000, a second miRNA, let-7, was reported to be an important miRNA in C. elegans development (Reinhart et al., 2000). Other miRNAs have been previously reported in various species, including worms, flies, and mammals. See, e.g., Chang et al., 2004; Johnston & Hobert, 2003; Brennecke & Cohen, 2003. See also the miRNA database accessible online at mirbase.org. Currently, more than two thousand miRNAs are known for the human genome and contained in miRBase Release 19 (Griffiths-Jones 2006).

miRNAs interact with their target RNAs in a sequence-dependent manner (Miranda et al., 2006; Bartel, 2004; Bartel, 2009; Rigoutsos et al., 2009). MiRNAs can function as post-transcriptional regulators of gene expression. In mammals, over 90% of all protein-coding genes are generally believed to be regulated by miRNAs (Miranda et al., 2006). Even though the entire miRNA sequence can bind to the mRNA target, the sequence spanning positions 2-7 inclusive from the 5′ end of a miRNA, known as the “seed,” is previously reported to be important for target determination and coupling (Bartel, 2004; Reinhart et al., 2000; Lee et al., 1993; Wightman et al., 1993; Tay et al., 2008; Brodersen, 2009; Lai et al., 2009; Rigoutsos, & Tsirigos, 2010 and Xia et al., 2012). Originally it was assumed that the presence of the reverse complement of the seed in mRNAs was both a sufficient and necessary condition for targeting (Bartel, 2009). However, genetic studies and numerous subsequent efforts showed miRNA functionality in the presence of inexact matches and/or bulges in the seed region (Wightman et al., 1993; Tay et al., 2008; Lai et al.; Ha et al., 1996; Vella et al., 2004; Farh et al., 2005; Stark et al., 2005; Didiano & Hobert, 2006; Easow et al., 2007; Baek et al., 2008; Selbach et al., 2008; Chi et al., 2009; Fabian et al., 2010; Hafner et al., 2010; Thomas 2010; Zisoulis et al., 2010; Chi et al., 2012 and Skalsky et al., 2012). The importance of such inexact matches has been previously reported to have direct relevance to answering the question of how many targets a miRNA can have.

The prevalence of miRNA-driven regulatory interactions across a very wide spectrum of human conditions and diseases makes the question of how many miRNAs existing a very important one. Recent advances in next generation sequencing have further complicated the picture by revealing that multiple distinct mature miRNA species can arise from the same miRNA precursor arm: these mature miRNAs are termed “isomiRs.” These isomiRs typically differ from the mature miRNA sequences currently in public databases such as miRBase (Griffiths-Jones 2006) on either their 5′ or 3′ ends thereby increasing the diversity and complexity of the miRNA-ome. While the biological relevance of isomiRs is not fully understood, they have been shown to associate with the Argonaute complex (Ameres & Zamore, 2013) which in turn suggests a functional role. Recent studies of isomiR expression have either focused on isomiRs of a single miRNA or on the isomiR expression patterns within a specific tissue. For example, a 5′-isomiR of miR-101 was observed to be ubiquitously expressed in several human tissues and cell lines (Llorens et al., 2013). There is a continuous demand for more accurate diagnostic and prognostic tools and improved therapies. As such, there is still a need to identify novel miRNA and isomiR sequences that can be used for diagnosis, prognosis, and therapy of human conditions or diseases or to identify the provenance (tissue or cell) of a biological sample.

Despite the discovery of thousands of novel miRNA transcripts located throughout the genome, the identification and quantification of miRNA transcripts originating from within polymorphic loci such as the MHC remains a significant challenge due to inherent sequence differences between the reference genome and the transcripts originating from any given individual. These differences are particularly problematic when mapping miRNA transcripts, since most miRNA mapping pipelines allow for no more than one mismatch between a sequenced read and the reference genome. Although stringent read mapping parameters are necessary to reduce the number of spuriously mapped reads, such an approach is inherently unable to align short reads that differ from the locus of origin within the reference genome by more than one base.

B. MHC

The gene dense MHC, located on the short arm of chromosome 6, is well known for the role its encoded genes play in numerous immunological processes. The clinical importance of this ˜4 Mbp region is emphasized by the maximal density of disease associated variants, as compared to any other 4 Mbp region of the human genome (Londin et al., 2015). However, despite the clinical importance of the MHC in human health and disease, the role of many disease associated variants within the MHC have yet to be fully elucidated, with ˜90% of causal autoimmune disease variants residing within non-coding regions of the human genome (Farh et al., 2015). Thus, the identification and characterization of functional non-coding elements within the MHC is a logical step towards elucidating the etiology of the numerous diseases associated with the MHC.

The MHC is known to encode numerous non-coding transcripts, including 12 annotated precursor miRNA hairpin (pre-miRNA) loci (Kozomara and Griffiths-Jones 2014). MicroRNAs (miRNAs) are a class of single stranded, non-coding RNA (ncRNA) transcripts, approximately 22 nucleotides in length that are known to attenuate the translation of targeted mRNA transcripts through the formation of a miRNA-mRNA heteroduplex, which is subsequently loaded onto the RNA induced silencing complex (RISC) (Jonas and Izaurralde 2015). Although the seminal database of annotated miRNA, miRBase (Kozomara and Griffiths-Jones 2014), contains entries for 2,813 mature human miRNA transcripts (database release 21), recent research has demonstrated the existence numerous novel, previously unannotated miRNA transcripts utilizing deep sequencing of short RNA transcripts (Jima et al., 2010; Meiri et al., 2010; Ladewig et al., 2012; Ple et al., 2012; Friedlander et al., 2014; Londin et al., 2015; Karali et al., 2016). Despite the advancement of miRNA discovery throughout the genome, the identification of novel miRNA within the MHC has lagged behind, due in part to the inability to accurately map short RNA read fragments originating from the highly variable MHC to the reference genome. The inherent sequence diversity present within the MHC makes mapping short read fragments to this particular portion of the reference genome difficult, especially since most mappers require only one mismatch between the read and the reference genome. New approaches to miRNA are therefore needed.

### SUMMARY

Thus, in accordance with the present disclosure, there is provided a method of designing an miRNA comprising (a) providing a target nucleic acid sequence; (b) generating in silico a population of putative pre-miRNA hairpin sequences having sequences of 58 to about 110 bases; (c) performing in silico folding of said putative pre-miRNA hairpin sequences to determine secondary structure and Gibbs minimum free energy (MFE); (d) filtering in silico said putative pre-miRNA hairpin sequences based on linearity of secondary structure and Gibbs MFE of less than about 20 Kcal/mol; (e) filtering in silico the putative pre-miRNA hairpin sequences remaining after step (d) to remove pre-miRNA hairpin sequences lacking resemblance with annotated putative pre-miRNA hairpin sequences from the genome of the target nucleic acid sequence; (f) filtering in silico the putative pre-miRNA hairpin sequences remaining after step (e) to remove sequences that overlap annotated exons of protein coding genes from the genome of the target nucleic acid sequence; and (g) merging overlapping the pre-miRNA hairpin sequences remaining after step (f) into a single locus.

The method may further comprise synthesizing at least one miRNA sequence from step (g). The method may further comprise introducing said at least one miRNA sequence into a cell, and assessing the effect of said at least one miRNA on expression of said target nucleic acid sequence. Assessing may comprise measuring transcript levels for said target nucleic acid sequence, measuring protein levels for the product encoded by said target nucleic acid sequence, measuring the activity of a protein encoded by said target nucleic acid sequence, determining the interaction of said at least one miRNA sequence with an miRNA-interacting molecule (e.g., Argonaute), or assessing the presence, absence or change of a pathologic phenotype. The pathologic phenotype may be a disease set forth in Table S3 and Table S4. The cell may be located in vitro, or in vivo. The cell may be a human cell or a non-human animal cell. The target nucleic acid sequence may be an MHC sequence.

In another embodiment, there is provided a method of determining whether a subject has, or is at risk of developing, or is at a given stage of a condition afflicting a tissue of interest, comprising measuring in a biological sample from the tissue of interest expression the level of one or more of the miRNAs, wherein the one or miRNAs comprise a sequence selected from SEQ ID NOS: 1-89, wherein the alteration of the level of said one or more miRNAs as compared to the level of the same one or more miRNA in a reference sample is indicative of the subject either having, or being at risk of developing, or is at a given stage of the condition. Isoforms of the miRNAs may be examined. The reference sample may represent a normal condition of the tissue, and/or a recognizable stage of an abnormal condition of the tissue. The expression level of 2, 3, 4, 5, 10, 15, 20, 25, 30, 40, 50, 60, 70, 80 or all 90 miRNAs may be measured.

In still another embodiment, there is provided a method of identifying a subject having or at risk of developing an immune or inflammatory disorder comprising (a) assessing the expression level of one or more miRNAs selected from SEQ ID NOS: 1-89 in a sample from said subject, and (b) comparing the expression level of said one or more miRNAs in said sample with a normal sample or predetermined control level, wherein an altered expression level of said one or more miRNAs indicates the existence of or increased risk for an immune or inflammatory disorder. The miRNA level may be elevated, reduced or a mixture of elevated and reduced. The sample may be a blood sample. The inflammatory disorder may be cancer, such as a cancer of the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. The immune disorder may be an autoimmune disorder, such as obesity, Crohn's disease, rheumatoid arthritis, asthma, autoimmune thyroid disease, blastic crisis, alopecia areata, multiple sclerosis, autoimmune hepatitis, Addison's disease, type 1 diabetes, type 2 diabetes, bladder cancer, chronic obstructive pulmonary disease, Grave's disease, systemic lupus erythematosus, lung cancer, or Alzheimer's disease, or may be IgA nephropathy or IgA deficiency. The subject may be a non-human animal or a human.

The method may further comprise treating a subject having or at risk of developing an immune or inflammatory disorder comprising administering to said subject an agonist or antagonist of an miRNA selected from SEQ ID NOS: 1-89. The antagonist may be a miR antagomir or antisense molecule. The agonist/antagonist may be formulated in a lipid delivery vehicle. The agonist/antagonist may be a nucleic acid containing at least one non-natural base. The agonist/antagonist may be administered multiple times. The agonist/antagonist may be administered daily, every other day, every third day, every fourth day, every fifth day, every sixth day, weekly or monthly. The agonist/antagonist may be administered continuously over a time period exceeding 24 hours.

In one aspect, provided herein relates to methods or assays of determining a given state of a cell and/or a tissue. The method or assay comprises detecting in a biological sample the presence or absence of one or more miRNAs described herein. In some embodiments, the biological sample can be derived from a subject suspected of being at risk of or having a given stage of a disease or disorder. Accordingly, methods or assays described herein can also be used to determine whether a subject has, or is at risk of developing, or is at a given stage of a disease or disorder, e.g., a condition afflicting a tissue of interest. In one embodiment, the condition afflicting a tissue of interest includes cancer.

In some embodiments, the method or assay described herein can comprise detecting in the biological sample the presence or absence of a plurality of the miRNAs described herein.

In some embodiments of the methods or assays described herein, detection of the presence or absence of at least one of the novel miRNAs described herein can include measuring an expression level of the miRNA of interest in the biological sample. The expression level of the miRNA of interest can be detected by any methods known in the art, including, but not limited to, sequencing, next-generation sequencing (e.g., deep sequencing), polymerase chain reaction (PCR), and real-time quantitative PCR, northern blot, microarray, in situ hybridization, serial analysis of gene expression (SAGE), cap analysis gene expression (CAGE), massively parallel signature sequencing (MPSS), direct multiplexed measurements of the type employed in the Nanostring platform, and any combinations thereof. In such embodiments, the methods or assays can further comprise comparing with a reference sample the determined expression level of the miRNA of interest in the biological sample. When there is a discrepancy in the expression level or amount of at least one miRNA molecule between the biological sample and the reference sample, the discrepancy can be indicative of the cell or the tissue in a state different from the reference sample. For example, in some embodiments, the discrepancy can be indicative of a subject either having, or being at risk of developing, or being at a given stage of a disease or disorder, e.g., a condition afflicting the tissue. In alternative embodiments, the discrepancy can be indicative of a subject lacking of a disease or disorder to be evaluated.

The reference sample used in the methods, assays and systems described herein can be a sample derived from the same type of cell or tissue with a known condition. For example, the reference sample can represent a normal condition of a cell or tissue to be detected. The normal reference sample can be obtained from the test subject or a different subject. Alternatively, the reference sample can represent a recognizable stage of a possibly abnormal condition of a cell or a tissue to be detected.

A biological sample for evaluation in the methods, assays, and systems described herein can include one or more cells derived from any tissue or fluid in a subject. In one embodiment, the biological sample can be a tissue suspected of being at risk of, or being afflicted with a given stage of a disease or a disorder. Non-limiting examples of sample origins can include, but are not limited to, breast, pancreas, blood, prostate, colon, lung, skin, brain, ovary, kidney, oral cavity, throat, cerebrospinal fluid, and liver.

Different embodiments of the methods, assays and systems described herein can be used for diagnosis and/or prognosis of a disease or disorder (including a given stage of a disease or disorder) in a subject, e.g., a disease or disorder afflicting a certain tissue in a subject. For example, the disease or disorder to be diagnosed and/or prognosed in a subject can be associated with breast, pancreas, blood, prostate, colon, lung, skin, brain, ovary, kidney, oral cavity, throat, cerebrospinal fluid, liver, and any combination thereof. In some embodiments, the disease or disorder to be diagnosed and/or prognosed with the methods, assays or systems described herein can be a blood disorder, e.g., associated with diseased or abnormal platelets. In other embodiments, the disease or disorder to be diagnosed and/or prognosed with the methods, assays or systems described herein can be any cancer, e.g., but not limited to breast cancer and pancreatic cancer.

For a subject who is determined to have, or is at risk of developing, or is at a given stage of the disease or disorder, the subject can be administered or prescribed with a specific treatment. For example, in some embodiments where the subject is diagnosed with cancer (e.g., breast carcinoma or pancreatic carcinoma) or progression thereof, the method can further comprise administering or prescribing the subject a treatment, e.g., chemotherapy, radiation therapy, surgery, engineered transcripts that can “sponge” various combinations of the novel miRNAs described herein, or any combinations thereof.

Another aspect provided herein relates to systems for analyzing a biological sample, e.g., to determine a given state of a cell or a tissue, and/or to diagnose and/or prognose a disease or disorder, or a given state of a disease or disorder in a subject. In one embodiment, the system comprises: (a) a determination module configured to receive a biological sample and to determine sequence information and, optionally quantity estimate information, wherein the sequence information comprises a sequence of one or more miRNAs described herein; and wherein the quantity estimate information comprises at least an estimate Of the abundance of said sequence, with said abundance optionally scaled with regard to the abundance of a reference molecule; (b) a storage device configured to store sequence information and optionally the quantity estimate information from the determination module; (c) a comparison module adapted to compare the sequence information and optionally the quantity estimate information stored on the storage device with reference data, and to provide a comparison result, wherein the comparison result identifies the presence or absence of the miRNA molecule, and optionally how its quantity estimate is related to the reference data, wherein a discrepancy in a quantity estimate level is indicative of the biological sample having an increased likelihood of having, or being at a cellular or tissue state different from a state represented by the reference data; and (d) a display module for displaying a content based in part on the comparison result for the user, wherein the content is a signal indicative of a subject having, or being at risk of developing, or being at a given stage of a disease or disorder, or a signal indicative of lacking a disease or disorder.

A computer-readable physical medium for determination of a given state of a cell or a tissue, including diagnosis and/or prognosis of a disease or disorder, or a state of a disease or disorder in a subject, is also provided herein. The computer-readable physical medium having computer readable instructions recorded thereon to define software modules includes a comparison module and a display module for implementing a method on a computer, wherein the method comprises: (a) comparing with the comparison module the data stored on a storage device with reference data to provide a comparison result, wherein the comparison result captures the presence or absence of the miRNA molecule and/or the difference between its quantity estimate and the reference data, wherein a discrepancy in a quantity estimate level is indicative of a biological sample having an increased likelihood of having, or being at a cellular or tissue state different from a state represented by the reference data; and (b) a display module for displaying a content based in part on the comparison result for the user, wherein the content is a signal indicative of a subject having, or being at risk of developing, or being at a given stage of a disease or disorder, or a signal indicative of lack of a disease or disorder.

In some embodiments, kits and/or assays for expressing, silencing, and/or quantitating one or more of the novel miRNAs described herein from the model organism of choice (e.g., but not limited to, human, mouse, and rat) are also encompassed within the scope of various aspects described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description of specific embodiments presented herein.

FIG. 1. Computational pipeline to discover novel miRNA expressed within two lymphoblastoid cell lines (PGF and COX). RNA-Seq was performed on two biological replicates of two homozygous BLCLs with completely characterized MHC haplotypes, PGF and COX. Mapped reads were utilized to discover significantly expressed novel mIRNA from each RNA-seq run using mirdeep*. In total 89, unique miRNA were discovered from all four datasets, with 87 of them having additional functional evidence.

FIG. 2. Sequence conservation of identified novel mature and pre-miRNA hairpin sequences across all known MHC haplotypes.

FIG. 3. Identified novel miRNAs that share sequence homology with annotated oncomiRs. The seed region of the miRNAs is defined by the red hashed line.

FIG. 4. Computational prediction pipeline and putative miRNA loci identified from the annotated MHC haplotype sequences of PGF and COX lymphoblastoid cell lines.

FIG. 5. Dicer was knocked-down in COX and PGF cells as described in materials and methods. Dicer expression was analyzed using q-PCR. Dicer expression in dicer knock-down cells is in reference to scrambled control set at 1. Dicer knock-down was tested 50 hours after first transduction. Cells were transduced for a total of 3 times as described in Methods. Cox dicer Scrambled Vs. knock-down T-test p=0.000222. PGF dicer scrambled vs. knock-down T-test p=0.001134

### DETAILED DESCRIPTION OF THE DISCLOSURE

As mentioned above, the inherent sequence diversity present within the MHC makes mapping short read fragments to this particular portion of the reference genome difficult, especially since most mappers require only one mismatch between the read and the reference genome. In order to overcome this limitation, the inventors have performed deep RNA sequencing of two homozygous lymphoblastoid cell lines, with completely characterized MHC haplotype sequences, PGF and COX (Stewart et al., 2004; Horton et al., 2008). This approach facilitates accurate mapping of short RNA-seq reads derived from PGF and COX to their respective reference MHC haplotype sequence, revealing haplotype specific expression patterns of novel miRNAs throughout the MHC, including the highly polymorphic HLA genes.

Given the limited availability of tissue samples derived from patients with completely characterized MHC haplotypes, and evidence demonstrating the tissue and phenotype dependent expression pattern of miRNAs (Fehlmann et al., 2016; Ludwig et al., 2016), the inventors' efforts to characterize the plethora of possible MHC encoded miRNAs through the analysis of RNA-seq data alone remains limited to the characterization of only those miRNA transcribed within two lymphoblastoid cell lines, PGF and COX. For this reason, the inventors employed a developed miRNA annotation pipeline using only the annotated MHC haplotype sequences of both PGF and COX as input. As a result, the they have generated an atlas of putative pre-miRNA encoding loci throughout the MHC.

Taken together, these efforts to characterize MHC derived miRNA transcripts lays the foundation for studying allele and haplotype specific miRNA expression patterns across sample populations with diverse MHC haplotypes, and the interpretation of disease associated variants throughout the MHC. Accordingly, provided herein relates to methods, compositions, assays and systems for determining a given state or identity of a cell and/or a tissue, and can be used for diagnosing a disease or disorder, and/or prognosing a given stage and/or progression of a disease or disorder, and/or treating a disease or disorder. The identified miRNAs can also be used to identify novel and unexpected molecular players in cellular contexts of interest and also to reveal unexpected molecular interplays that underlie the onset and progression of diseases. The miRNAs may also be used for improved means for therapeutic interventions.

Thus, by analyzing short RNA sequence profiles of sequenced material from various human tissues and/or cell types using their particular analytic methods, the inventors have demonstrated that miRNA identification/comparison is not only possible, but can be applied to a variety of targets, cells, tissues and organisms. These and other aspects of the disclosure are set out in detail below.

### I. Methods and Assays for Determining a Given State of a Cell or Tissue

One aspect described herein provides methods and assays for determining a specific state or condition of a cell or a tissue. As the cell or tissue can be derived from a biological sample of a subject suspected of being at risk of or having a given stage of a disease or disorder, e.g., a condition afflicting a tissue, methods and assays for determining whether a subject has or is at risk of developing, or is at a given stage of a disease or disorder, e.g., a condition afflicting a tissue, are also provided herein. The methods or assays of any aspects described herein comprise measuring in a biological sample from a tissue of interest expression level of one or more miRNAs disclosed herein. In some embodiments, the methods or assays of any aspects described herein comprise measuring in a biological sample from a tissue of interest expression level of one or more miRNAs disclosed herein. An alteration of the level of one or more miRNAs as compared to the level of the same miRNA sequence(s) in a reference sample is indicative of the subject either having, or being at risk of developing, or being at a given stage of the condition.

As shown in the Examples, some of the novel miRNAs/isomiRs are preferentially present in some tissues but not in other tissues. This indicates that signatures of one or more miRNAs can be used to answer the question of tissue of origin. Thus, in some embodiments, the methods and/or assays described herein can also be used to determine tissue of origin by comparing the level(s) of one or more miRNAs in the biological sample to that of reference samples of different tissues.

As used herein, the term “alteration of the level of one or more miRNAs” refers to a change in the level of one or more miRNAs in a sample relative to the corresponding level(s) in a reference sample. In some embodiments, the alteration or change in the level of one or more miRNAs can refer to an increase in the level of one or more miRNAs in a sample relative to the corresponding level(s) in a reference sample. For example, in some embodiments where there is an alteration in the level of one or more miRNAs, the level of one or more miRNAs in a sample can be increased by at least about 10% or more, including, e.g., at least about 20%, at least about 30%, at least about 40%, at least about 50%, at least about 60%, at least about 70%, at least about 80%, at least about 90%, at least about 95%, at least about 97%, at least about 99% or more, relative to the corresponding level(s) in a reference sample. In some embodiments where there is an alteration in the level of one or more miRNAs, the level of one or more miRNAs in a sample can be increased by at least about 1.1-fold or more, including, e.g., at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold or more, relative to the corresponding level(s) in a reference sample.

In some embodiments, the level(s) of one or more miRNAs are considered to be altered (or differentially expressed) relative to reference sample(s) if it has a mean expression of at least 25 or more sequenced reads, and a log 2-change in expression relative to the reference sample(s) of at least 0.5 or higher, or no more than −0.5 or lower.

In other embodiments, the alteration or change in the level of one or more miRNAs can refer to a decrease in the level of one or more miRNAs in a sample relative to the corresponding level(s) in a reference sample. For example, in some embodiments where there is an alteration in the level of one or more miRNAs, the level of one or more miRNAs in a sample can be decreased by at least about 10% or more, including, e.g., at least about 20%, at least about 30%, at least about 40%, at least about 50%, at least about 60%, at least about 70%, at least about 80%, at least about 90%, at least about 95%, at least about 97%, at least about 99% or more (including 100%), relative to the corresponding level(s) in a reference sample.

In some embodiments, the “alteration of the level of one or more miRNAs” can refer to the presence (e.g., a detectable level) of one or more miRNAs in a sample, as compared to the absence (e.g., no detectable level) of those miRNA(s) in a reference sample.

In alternative embodiments, the “alteration of the level of one or more miRNAs” can refer to the absence (e.g., no detectable level) of one or more miRNAs in a sample, as compared to the presence (e.g., a detectable level) of those miRNA(s) in a reference sample.

In some embodiments of any aspects described herein, the method or assay can comprise detecting in the biological sample the level of a plurality (e.g., at least 2, at least 3, at least 4, at least 5, at least 6, at least 7, at least 8, at least 9, at least 10, at least 15, at least 20, at least 25, at least 30, at least 40, at least 50, or more) of the miRNAs disclosed herein.

An amount of a miRNA sequence in the biological sample can be measured or quantified by any known RNA detection methods. By way of example only, the miRNA(s) in a biological sample can be detected or read by a sequencing method (including Sanger sequencing, next-generation sequencing or deep sequencing, direct multiplexing, and any art-recognized sequencing method) and a read count of each miRNA sequence can be generated to determine its amount present in the biological sample. Alternatively, where the miRNA sequence(s) in a biological sample are determined by PCR-based methods (e.g., real-time PCR), the amount of the miRNA sequence(s) present in the biological sample can be represented by a Cₜ number, which can be compared to that of a reference sample. As a person having ordinary skill in the art would appreciate, a larger Cₜ number generally indicates a lower amount of a nucleic acid sequence present in a sample. In some embodiments, the quantitative amount of the miRNA sequence(s) detected by PCR-based methods (e.g., real-time PCR) can also be determined from a calibration curve generated with known amounts of a nucleic acid sequence.

Any other art-recognized methods detecting or measuring the level of a miRNA sequence, e.g., but not limited to the methods and kits for miRNA isolation and quantitation as described in U.S. Pat. No. 8,574,838, can also be used herein. Additional information about measuring or quantifying one or more miRNA sequences is described in the section “Exemplary methods for detecting or measuring the level of a miRNA sequence” below.

As the amount of the miRNA sequence(s) is determined in the biological sample, in some embodiments, the methods or assays described herein can further comprise comparing with a reference sample the level of one or more miRNA sequences described herein in the biological sample. When there is a difference (e.g., at least about 10% difference or higher) or a statistically significant difference in an amount of at least one or more (e.g., at least 2 or more) or in the profile of miRNA sequences between the biological sample and the reference sample, the difference or significant difference can be indicative of the cell or the tissue in a state different from the reference sample. If the cell or the tissue is derived from a biological sample of a subject, the results of the comparison can be used for diagnosing or prognosing a disease or disorder, or a state of a disease or disorder. Depending on the choice of a reference sample, in some embodiments, the difference or significant difference can be indicative of a subject either having, or being at risk of developing, or being at a given stage of a disease or disorder, e.g., a condition afflicting the tissue; while in other embodiments, the difference or significant difference can be indicative of a subject free of a disease or disorder, e.g., a condition afflicting the tissue. For example, if a subject's miRNA sequence level or profile has no significant difference from that of a normal sample, it indicates that the subject is free of a condition or disease. Additionally or alternatively, if a subject's miRNA sequence level or profile is significantly different from that of a sample of a known disease or disorder to be diagnosed, it indicates that the subject does not likely have that disease or disorder.

If the miRNA sequence level or profile of a biological sample contain miRNA(s) that are preferentially present in a certain tissue but not in other tissues, the tissue origin of the biological sample can be determined based on the presence of those tissue-specific miRNA(s).

The threshold level selected to distinguish a given state of a cell or tissue from another, and/or to determine if a subject has, or is at risk of developing, or is at a given stage of a condition afflicting a tissue of interest can be determined experimentally. For example, by comparing the levels and/or profiles of one or more miRNA molecules detected in a number of references samples of known conditions in a specific tissue (e.g., a normal breast sample vs. a cancerous breast sample), e.g., by deep sequencing and/or quantitative RT-PCR, one of skill in the art can determine a threshold level for levels of one or more miRNA sequences required to distinguish one condition from another (e.g., to distinguish a normal breast sample from a cancerous breast sample).

In some embodiments, the threshold level needs not be pre-determined. For example, cluster analysis can be computationally performed to classify miRNA expression profiles of a biological sample and a set of reference samples into groups such that samples in the same group (called a cluster) have more similar miRNA expression profiling to each other than to those in other groups (clusters). Accordingly, when a biological sample is categorized into the same group with a subset of reference samples of similar miRNA expression profiles, the biological sample is considered to have similar properties (e.g., genotype or phenotype) as the subset of reference samples. For example, when a breast tissue sample from a subject is categorized into the same group representing cancerous breast tissue reference samples, or more specifically, into a sub-group representing invasive breast cancer, the subject is determined to likely to have breast cancer, or more specifically an invasive breast cancer. Various clustering algorithms for classification and clustering are known in the art and can be used for the purposes described herein. Examples of clustering algorithms and/or models include, but are not limited to connectivity-based clustering (e.g., hierarchical clustering), centroid-based clustering (e.g., k-means clustering), distribution-based clustering (e.g., multivariate normal distributions used by the expectation-maximization algorithm), density-based clustering (e.g., DBSCAN and OPTICS), subspace models (e.g., biclustering), and any combinations thereof.

The reference sample used in the methods and assays described herein can be a sample derived from the same type of cell or tissue as a biological sample, and with a known condition. For example, the reference sample can represent a normal condition of a cell or tissue in a biological sample to be analyzed. The normal reference sample can be collected from a subject whose biological sample is being analyzed or from a different subject. Alternatively, the reference sample can represent a recognizable stage of an abnormal condition of a cell or a tissue in a biological sample to be analyzed. By way of example only, if a disease or disorder to be diagnosed or prognosed in a subject is breast cancer, a reference sample can include a normal breast tissue, a ductal carcinoma in situ breast tissue sample, an invasive ductal carcinoma tissue sample or subtype, an invasive lobular carcinoma tissue sample, a lobular carcinoma in situ tissue sample, and any combinations thereof.

In some embodiments, the reference sample can present a known condition of a known tissue origin. Depending on applications, the reference sample can have the same or different tissue origin from the biological sample. For example, in order to determine the tissue origin of a biological sample, the level(s) and/or profile of one or more miRNAs described herein can be compared to reference samples of different tissues.

By way of example only, in some embodiments, the methods described herein can be used to determine a primary origin of an unknown tumor or cancer. Thus, in some embodiments, the methods described herein can be used to determine whether the tumor is a primary tumor or a secondary tumor (i.e., a metastasis). For example, a biopsy of an unknown tumor can be subjected to the methods or assays described herein to determine the tissue origin of the tumor, wherein if the tissue origin of the tumor is determined to be the same tissue type as from where the biopsy is collected, the tumor is diagnosed as a primary tumor, or if the tissue origin of the tumor is determined to be different from the type of the tissue from where the biopsy is collected, the tumor is diagnosed as a secondary tumor (i.e., a metastasis). Thus, the methods described herein can be used to fingerprint a biological sample, e.g., whether it is a normal sample or a diseased sample (e.g., a cancerous sample).

In some embodiments, more than one reference samples can be used, wherein the reference samples can represent a variety of different conditions (e.g., normal condition, different stages of a disease or disorder, different tissue origins). By way of example only, if a biological sample of a subject generates a similar or comparable miRNA expression profile (e.g., in terms of levels and/or locations of miRNA sequences) to that of a normal sample or a group of normal samples (reference samples), the subject can be considered normal with respect to the normal sample or the group normal samples. Similarly, if a biological sample of a subject generates a similar or comparable miRNA expression profile (e.g., in terms of levels and/or locations of miRNA sequences) to that of a sample or a group of samples derived from a given tissue (reference samples), the subject's biological sample can be considered to have the same tissue type as the reference samples.

The miRNA expression profile can be generated based on one or more genes. In some embodiments, the miRNA expression profile can be generated based on a specific gene, e.g., a gene that is associated with a condition or disorder. In some embodiments, miRNA sequences can be detected from at least two genes in a biological sample and compared to the corresponding reference sample to determine if similar conclusions are obtained.

For a subject who is determined to have, or is at risk of developing, or is at a given stage of the disease or disorder, the subject can be administered or prescribed with an appropriate treatment. For example, in some embodiments where the subject is diagnosed with cancer (e.g., breast carcinoma or pancreatic carcinoma) or progression thereof, the method can further comprise administering or prescribing the subject an anti-cancer treatment, e.g., chemotherapy, radiation therapy, surgery, immunotherapy, RNA therapeutics (e.g., siRNAs, miRNAs), or any combinations thereof.

In some embodiments, a subject in need thereof can be administered with an antagonist or a mimic of one or more (e.g., at least one, at least two, at least three, at least four, at least five, or more) of the miRNA disclosed herein, depending on whether the subject is diagnosed to have an overexpression or a deficiency of one or more miRNA sequences determined to be associated with a condition or disorder. For example, in some embodiments where the level(s) of one or more miRNAs described herein are determined to represent a “causal event” for the disease or disorder, the level(s) of the miRNAs can be controlled in order to return their levels to what would be considered “normal” levels and thus alleviate the impact that can result from the changes in their amount. Examples of the techniques that can be used to control the level(s) of one or more miRNAs described herein include, but are not limited to, antisensing or sponging (e.g., microRNA sponges as described in Ebert & Shape, 2010 and Ebert et al., Nat. Methods, 4: 721-726, 2007, decoying (e.g., as described in Swami, Nature Reviews Genetics, 11:530-531, 2010), overexpression, and/or any art-recognized techniques.

Additional information about antagonists or mimics of miRNAs described herein is described in the section “Pharmaceutical Compositions” below.

### II. miRNA Sequences

The inventors have discovered a collection of novel microRNAs (miRNAs) in a human genome, based on deep sequencing data using different tools and/or design methodology. The cardinality of the class of non-coding RNAs (ncRNAs) known as microRNAs or miRNAs has been a controversial issue for more than a decade. It was previously estimated that the number of human miRNAs would not exceed 300 (Lim et al., 2003). However, different methods that were used later provided higher, yet concordant, figures for the number of human miRNAs and their cardinality was estimated to be in the several tens of thousands. The miRBase repository has served as a widely-accepted public clearinghouse of miRNA sequences for vertebrates, invertebrates, viruses and plants.

Novel miRNAs are provided herein. Oligonucleotides comprising sequences complementary to the novel miRNAs are also encompassed within the scope of the disclosures described herein. By “complementary” is meant that an oligonucleotide can form hydrogen bond(s) with another oligonucleotide by either traditional Watson-Crick or other non-traditional types. The complementary oligonucleotides can be completely or partially complementary to the novel miRNA sequences. In some embodiments, partial complementarity is indicated by the percentage of contiguous residues in an oligonucleotide that can form hydrogen bonds (e.g., Watson-Crick base pairing) with a second oligonucleotide sequence (e.g., 5, 6, 7, 8, 9, 10 out of 10 being 50%, 60%, 70%, 80%, 90%, and 100% complementary). “Completely complementary” or 100% complementarity means that all the contiguous residues of an oligonucleotide sequence will form hydrogen bond with the same number of contiguous residues in a second oligonucleotide sequence. Less than perfect complementarity refers to the situation in which some, but not all, nucleotide units of two strands can hydrogen bond with each other.

In some instances, both the “miRNA” and the “isomiR” can be found in the same tissue but the isomiR has a lower expression than the miRNA. In some instances, tissues where what is commonly called as “miRNA” has lower expression than an “isomiR” from the same locus: in such examples, the labels of miRNA and isomiR can be used interchangeably. Accordingly, as used herein, the terms “miRNA” and “isomiR” are used herein interchangeably and will be making a distinction only if necessary to remove any ambiguities.

While the novel miRNA sequences disclosed herein are from human genomes, many of these sequences are conserved in other species, e.g., but not limited to other primates such as Chimpanzee, Gorilla, Orangutan, and Macaque. Some human miRNA sequences as described herein are also conserved in mice, fruit-fly, and worms.

MicroRNAs (miRNAs) are small, non-coding RNA molecules that generally function as regulators of a genome. The terms “microRNA” or “miRNA” are used interchangeably herein, are generally endogenous RNAs, some of which are known to regulate the expression of protein-coding genes at the posttranscriptional level. As used herein, the term “microRNA” refers to any type of micro-interfering RNA, including but not limited to, endogenous microRNA and artificial or synthetic microRNA. Typically, endogenous microRNA are small RNAs encoded in the genome which are capable of modulating the productive utilization of mRNA. A mature miRNA is a single-stranded RNA molecule of about 21-23 nucleotides in length that is complementary to a target sequence and hybridizes to the target RNA sequence to inhibit expression of a (protein-coding or non-coding) gene which encodes a miRNA target sequence. miRNAs themselves are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression. MicroRNA sequences have been described in publications such as, (Lim, et al., 2003a; Lim et al., 2003b; Lee & Ambros, 2001; Lau et al., 2001); Lagos-Quintana et al., 2002; Lagos-Quintana et al., 2001 and Lagos-Quintana et al., 2003), which are incorporated by reference. Multiple microRNAs can also be incorporated into the precursor molecule.

A mature miRNA is produced as a result of a series of miRNA maturation steps; first a gene encoding the miRNA is transcribed. The gene encoding the miRNA is typically much longer than the processed mature miRNA molecule; miRNAs are first transcribed as primary transcripts or “pri-miRNA” with a cap and poly-A tail, which is subsequently processed to short, about 70-nucleotide “stem-loop structures” known as “pre-miRNA” in the cell nucleus. This processing is performed in animals by a protein complex known as the Microprocessor complex, consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). This complex is responsible for the gene silencing observed due to miRNA expression and RNA interference. The pathway is different for miRNAs derived from intronic stem-loops; these are processed by Drosha but not by Dicer. In some instances, a given region of DNA and its complementary strand can both function as templates to give rise to at least two miRNAs. Mature miRNAs can direct the cleavage of mRNA or they can interfere with translation of the mRNA, either of which results in reduced protein accumulation, rendering miRNAs capable of modulating gene expression and related cellular activities.

As discussed above, recent advances in next generation sequencing have complicated this picture by revealing that multiple distinct mature miRNA species, the isomiRs, can arise from the same miRNA precursor arm (“pre-miR”). The isomiRs typically differ from the mature miRNA sequences currently in public databases such as miRBase on either their 5′ or 3′ ends, thereby increasing the diversity and complexity of the miRNA-ome. IsomiRs, just like miRNAs, associate with the Argonaute complex. Accordingly, the terms “miRNA” and “isomiR” are used herein interchangeably and will be making a distinction only if necessary to remove any ambiguities.

The term “pri-miRNA” refers to a precursor to a mature miRNA molecule which comprises: (i) a microRNA sequence and (ii) stem-loop component which are both flanked (i.e., surrounded on each side) by “microRNA flanking sequences,” where each flanking sequence typically ends in either a cap or poly-A tail. A pri-microRNA, (also referred to as large RNA precursors), are composed of any type of nucleic acid based molecule capable of accommodating the microRNA flanking sequences and the microRNA sequence. Examples of pri-miRNAs and the individual components of such precursors (flanking sequences and microRNA sequence) are provided herein. The nucleotide sequence of the pri-miRNA precursor and its stem-loop components can vary widely. In one embodiment, a pre-miRNA molecule can be an isolated nucleic acid; including microRNA flanking sequences and comprising a stem-loop structure and a microRNA sequence incorporated therein. A pri-miRNA molecule can be processed in vivo or in vitro to an intermediate species caller “pre-miRNA,” which is further processed to produce a mature miRNA.

The term “pre-miRNA” refers to the intermediate miRNA species in the processing of a pri-miRNA to mature miRNA, where pri-miRNA is processed to pre-miRNA in the nucleus, whereupon pre-miRNA translocates to the cytoplasm where it undergoes additional processing in the cytoplasm to form mature miRNA. Pre-miRNAs are generally about 70 nucleotides long, but can be less than 70 nucleotides or more than 70 nucleotides.

The term “microRNA flanking sequence” as used herein refers to nucleotide sequences including microRNA processing elements. MicroRNA processing elements are the minimal nucleic acid sequences which contribute to the production of mature miRNA from precursor microRNA. Often these elements are located within a 40 nucleotide sequence that flanks a microRNA stem-loop structure. In some instances the microRNA processing elements are found within a stretch of nucleotide sequences of between 5 and 4,000 nucleotides in length that flank a microRNA stem-loop structure. Thus, in some embodiments the flanking sequences are 5-4,000 nucleotides in length. As a result, the length of the precursor molecule can be, in some instances at least about 150 nucleotides or 270 nucleotides in length. The total length of the precursor molecule, however, can be greater or less than these values. In other embodiments the minimal length of the microRNA flanking sequence is 10, 20, 30, 40, 50, 60, 70, 80, 90, 100, 150, 200 and any integer there between. In other embodiments the maximal length of the microRNA flanking sequence is 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900 4,000 and any integer there between.

MicroRNA flanking sequences can be native microRNA flanking sequences or artificial microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Artificial microRNA flanking sequences are nucleotides sequences that are not found to be flanking to microRNA sequences in naturally existing systems. miRNA flanking sequences within the pri-miRNA molecule can flank one or both sides of the stem-loop structure encompassing the microRNA sequence. Thus, one end (i.e., 5′) of the stem-loop structure can be adjacent to a single flanking sequence and the other end (i.e., 3′) of the stem-loop structure cannot be adjacent to a flanking sequence. Preferred structures have flanking sequences on both ends of the stem-loop structure. The flanking sequences can be directly adjacent to one or both ends of the stem-loop structure or can be connected to the stem-loop structure through a linker, additional nucleotides or other molecules.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the disclosure as long as the secondary structure is present. As is known in the art, the secondary structure does not require exact base-pairing. Thus, the stem can include one or more base mismatches. Alternatively, the base-pairing can be exact, i.e., not include any mismatches. In some instances the precursor microRNA molecule can include more than one stem-loop structure. The multiple stem-loop structures can be linked to one another through a linker, such as, for example, a nucleic acid linker or by a microRNA flanking sequence or other molecule or some combination thereof.

Furthermore, miRNA-like stem-loops can be expressed in cells as a vehicle to deliver artificial or synthetic miRNAs for the purpose of modulating the expression of endogenous genes through the miRNA and/or RNAi pathways.

In some embodiments, the miRNAs described herein are miRNA mimetic. As used herein, the term “miRNA mimetic” refers to an artificial miRNA which is flanked by the appropriate sequences that will allow it to form the stem-loop like structures that are typical of a pri-miRNA. The term “artificial microRNA” includes any type of RNA sequence, other than endogenous microRNA, which is capable of modulating the productive utilization of mRNA, For instance, the term artificial microRNA also encompasses a nucleic acid sequence which would be previously identified as siRNA, where the siRNA is incorporated into a vector and surrounded by miRNA flanking sequences as described herein.

In some embodiments, the term “miRNA” as used herein can encompass an oligonucleotide having a hairpin sequence and a mature miRNA sequence. In some embodiments, the term “miRNA” can encompass isomiR.

In some embodiments, the novel miRNA sequences described herein can encompass at least one or more nucleotide modifications, e.g., addition, deletion and/or substitutions of nucleotides, and/or modifications to the side groups and/or backbone of the nucleotides. Unmodified miRNA sequences can be less than optimal in some applications, e.g., unmodified miRNA sequences can be prone to degradation by, e.g., cellular nucleases. However, chemical modifications to one or more of the subunits of the miRNA sequences can confer improved properties, e.g., can render miRNA sequences more stable to nucleases. Typical miRNA sequence modifications can include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone, e.g., peptide nucleic acid (PNA); (vi) modification of the 3′ end or 5′ end of the oligonucleotide, e.g., removal, modification or replacement of a terminal phosphate group or conjugation of a moiety, e.g., conjugation of a ligand, to either the 3′ or 5′ end of oligonucleotide; and (vii) modification of the sugar, e.g., six membered rings.

The terms “replacement,” “modification,” “alteration,” and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule. As described below, modifications, e.g., those described herein, can be provided as asymmetrical modifications.

Diseases or disorders that can be diagnosed, prognosed, and/or treated using one or more novel miRNAs described herein or complementary sequences thereof.

In some aspects, different embodiments of the methods, assays, compositions, and systems described herein can be used for diagnosis and/or prognosis of a disease or disorder, and/or the state of the disease or disorder in a subject, e.g., a condition afflicting a certain tissue in a subject. In other aspects, different compositions described herein can be used for treatment of a disease or disorder in a subject. For example, the disease or disorder in a subject can be associated with breast, pancreas, blood, prostate, colon, lung, skin, brain, ovary, kidney, oral cavity, throat, cerebrospinal fluid, liver, or other tissues, and any combination thereof.

In some embodiments, diseases or disorders that can be diagnosed, prognosed, and/or treated using one or more novel miRNAs described herein or complementary sequences thereof can include conditions that are not terminal but can cause an interruption, disturbance, or cessation of a bodily function, system, or organ. Such examples of diseases or disorders can include, e.g., but are not limited to developmental disorders (e.g., autism), brain disorders (e.g., epilepsy), mental disorders (e.g., depression), endocrine disorders (e.g., diabetes), heart diseases (e.g., cardiomyopathy), or skin disorders (e.g., skin inflammation).

In some embodiments, diseases or disorders that can be diagnosed, prognosed, and/or treated using one or more novel miRNAs described herein or complementary sequences thereof can include breast diseases or disorders. Exemplary breast disease or disorder includes breast cancer.

In some embodiments, diseases or disorders that can be diagnosed, prognosed, and/or treated using one or more novel miRNAs described herein or complementary sequences thereof can include pancreatic diseases or disorders. Non-limiting examples of pancreatic diseases or disorders include acute pancreatitis, chronic pancreatitis, hereditary pancreatitis, pancreatic cancer (e.g., endocrine or exocrine tumors), etc., and any combinations thereof.

In some embodiments, diseases or disorders that can be diagnosed, prognosed, and/or treated using one or more novel miRNAs described herein or complementary sequences thereof can include blood diseases or disorders. Examples of blood diseases or disorders include, but are not limited to, platelet disorders, von Willebrand diseases, deep vein thrombosis, pulmonary embolism, sickle cell anemia, thalassemia, anemia, aplastic anemia, fanconi anemia, hemochromatosis, hemolytic anemia, hemophilia, idiopathic thrombocytopenic purpura, iron deficiency anemia, pernicious anemia, polycythemia vera, thrombocythemia and thrombocytosis, thrombocytopenia, and any combinations thereof.

In some embodiments, diseases or disorders that can be diagnosed, prognosed, and/or treated using one or more novel miRNAs described herein or complementary sequences thereof can include prostate diseases or disorders. Non-limiting examples of prostate diseases or disorders can include prostatis, prostatic hyperplasia, prostate cancer, and any combinations thereof.

In some embodiments, diseases or disorders that can be diagnosed, prognosed, and/or treated using one or more novel miRNAs described herein or complementary sequences thereof can include colon diseases or disorders. Exemplary colon diseases or disorders can include, but are not limited to, colorectal cancer, colonic polyps, ulcerative colitis, diverticulitis, and any combinations thereof.

In some embodiments, diseases or disorders that can be diagnosed, prognosed, and/or treated using one or more novel miRNAs described herein or complementary sequences thereof can include lung diseases or disorders. Examples of lung diseases or disorders can include, but are not limited to, asthma, chronic obstructive pulmonary disease, infections, e.g., influenza, pneumonia and tuberculosis, and lung cancer.

In some embodiments, diseases or disorders that can be diagnosed, prognosed, and/or treated using one or more novel miRNAs described herein or complementary sequences thereof can include skin diseases or disorders, or skin conditions. An exemplary skin disease or disorder can include skin cancer, e.g., melanoma; and psoriasis.

In some embodiments, diseases or disorders that can be diagnosed, prognosed, and/or treated using one or more novel miRNAs described herein or complementary sequences thereof can include brain diseases or disorders. Examples of brain diseases or disorders can include, but are not limited to, brain infections (e.g., meningitis, encephalitis, brain abscess), brain tumor, glioblastoma, stroke, ischemic stroke, multiple sclerosis (MS), vasculitis, and neurodegenerative disorders (e.g., Parkinson's disease, Huntington's disease. Pick's disease, amyotrophic lateral sclerosis (ALS), dementia, and Alzheimer's disease), and any combinations thereof.

In some embodiments, diseases or disorders that can be diagnosed, prognosed, and/or treated using one or more novel miRNAs described herein or complementary sequences thereof can include liver diseases or disorders. Examples of liver diseases or disorders can include, but are not limited to, hepatitis, cirrhosis, liver cancer, biliary cirrhosis, primary sclerosing cholangitis, Budd-Chiari syndrome, hemochromatosis, transthyretin-related hereditary amyloidosis, Gilbert's syndrome, and any combinations thereof.

In other embodiments, diseases or disorders that can be diagnosed, prognosed, and/or treated using one or more novel miRNAs described herein or complementary sequences thereof can include cancer. Examples of cancers can include, but are not limited to, bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS associated leukemias and adult T-cell leukemia lymphoma; intraepithelial neoplasms including Bowen's disease and Paget's disease, liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilm's tumor.

In some embodiments, the methods, assays, kits, and systems described herein can be used for determining a given stage of cancer in a subject. The stage of a cancer generally describes the extent the cancer has progressed and/or spread. The stage usually takes into account the size of a tumor, how deeply the tumor has penetrated, whether the tumor has invaded adjacent organs, how many lymph nodes the tumor has metastasized to (if any), and whether the tumor has spread to distant organs. Staging of cancer is generally used to assess prognosis of cancer as a predictor of survival, and cancer treatment is primarily determined by staging. Thus, methods, systems, kits, and assays for determining a given stage of cancer in a subject are also provided herein. For example, such methods and assays can comprise detecting in a biological sample (e.g., a biopsy) the presence, absence or level of one or more miRNA sequences described herein.

In some embodiments, the cancer to be diagnosed or prognosed can be breast carcinoma. In such embodiments, the methods or assays described herein can be used to distinguish a cancerous breast tissue from a normal breast tissue, or identify a given stage of a cancerous breast tissue, e.g., ductal carcinoma in situ, lobular carcinoma in situ, invasive ductal carcinoma or a subtype, invasive lobular carcinoma, etc.

In some embodiments, the cancer to be diagnosed or prognosed can be pancreatic cancer. In such embodiments, the methods or assays described herein can be used to distinguish a cancerous pancreas tissue from a normal pancreas tissue, or identify a given state of a cancerous pancreas tissue, e.g., early-stage pancreatic cancer or late-stage pancreatic cancer.

In some embodiments, the methods, assays, kits, and systems described herein can be used for determining tissue origin of a biological sample from a subject. For example, the tissue origin of a biological sample from a subject can be determined based on comparison of the level(s) and/or profile of one or more miRNAs described herein in the biological sample with reference samples of different tissues.

By way of example only, in some embodiments, the methods, assays, kits, and systems described herein can be used to determine a primary origin of an unknown tumor or cancer. Thus, in some embodiments, the methods described herein can be used to determine whether the tumor is a primary tumor or a secondary tumor (i.e., a metastasis). For example, a biopsy of an unknown tumor can be subjected to the methods or assays described herein to determine the tissue origin of the tumor, wherein if the tissue origin of the tumor is determined to be the same tissue type as from where the biopsy is collected, the tumor is diagnosed as a primary tumor, or if the tissue origin of the tumor is determined to be different from the type of the tissue from where the biopsy is collected, the tumor is diagnosed as a secondary tumor (i.e., a metastasis). Thus, the methods described herein can be used to fingerprint a biological sample, e.g., whether it is a normal sample or a diseased sample (e.g., a cancerous sample).

For a subject who is determined to have, or is at risk of developing, or is at a given stage of cancer (e.g., breast carcinoma or pancreatic carcinoma), the subject can be administered or prescribed with an anti-cancer treatment, e.g., chemotherapy, radiation therapy, surgery, immunotherapy, RNA therapeutics (e.g., siRNAs, miRNAs), or any combinations thereof.

In some embodiments, a subject in need thereof can be administered with an antagonist or a mimic of one or more of the miRNA sequences disclosed herein, depending on whether the subject is diagnosed to have an overexpression or a deficiency of one or more miRNA sequences determined to be associated with a condition or disorder. Additional information about antagonists or mimics of miRNAs described herein is described in the section “Pharmaceutical Compositions” below.

### III. Exemplary Methods for Detecting Target and miRNA Sequences

One or more nucleic acid sequence(s) can be determined/detected by any methods known in the art, including, but not limited to, Sanger sequencing, nucleic acid amplification (e.g., polymerase chain reaction (PCR), and real-time quantitative PCR), northern blot, nucleic acid hybridization (e.g., microarray), in situ hybridization, serial analysis of gene expression (SAGE), cap analysis gene expression (CAGE) and massively parallel signature sequencing (MPSS), next generation sequencing (including deep sequencing, e.g., sequencing with deep coverage), direct multiplexing, etc., and any combinations thereof.

Methods for performing SAGE to detect RNAs have been previously described in Velculescu et al., 1995 and Saha et al., 1997 and exemplary SAGE protocols can be accessed at sagenet.org/protocollindex.htm. Methods for performing CAGE to detect RNAs has been previously described, e.g., in Kodzius et al., 2006. Methods for performing MPSS to detect RNAs can be found, e.g., in Brenner et al., 2000.

In some embodiments, the INVADER® assay (Third Wave Technologies Inc., Madison, Wis.) can be modified and used to detect miRNA sequences in a biological sample. The INVADER® assay is generally a homogeneous, isothermal, signal amplification system for the quantitative detection of nucleic acids. The assay can directly detect either DNA or RNA without target amplification or reverse transcription. It is based on the ability of Cleavase® enzymes to recognize as a substrate and cleave a specific nucleic acid structure generated through the hybridization of two oligonucleotides to the target sequence. Modification of the INVADER® assay for miRNA sequence detection has been previously described, e.g., in de Arruda et al., 2002; Eis et al., 2001; and Allawi et al., 2004.

Next-generation sequencing (NGS) is a novel approach for the detection and sequencing of DNA or RNA molecules as reviewed, e.g., in Voelkerding et al., 2009; Metzker 2010; Zhang et al., 2011 and Pareek et al., 2011. Various commercial NGS instruments and reagent kits for high-throughput next-generation sequencing have been developed and used for RNA-sequencing. For example, exemplary NGS instruments that can be used for RNA-sequencing or deep sequencing of RNA can include, but are not limited to, the GS FLX sequencer (based on pyrosequencing) from 454 Life Sciences now part of ROCHE Diagnostics (world-wide-web at 454.com/), the Genome Analyzer (based on polymerase-based sequence-by-synthesis) from Illumina (world-wide-web at illumina.com), the SOLiD™ System (based on ligation-based sequencing) from Applied Biosystems (world-wide-web at appliedbiosystems.com/absite/us/en/home/applications-technologies/solid-next-generation-sequencing/next-generation-systems.html), and the HeliScope™ Single Molecule Sequencer from Helicos Bioscience (world-wide-web at helicosbio.com/).

Other NGS or higher-generation sequencing methods based on single-molecule sequencing (without PCR amplification) can also be used to detect miRNA sequences or molecules in some embodiments of the methods, assays, and systems described herein. Examples of single-molecule sequencing methods can include, but are not limited to, Ion Torrent (pH sensing), nanopore sequencing, and transmission electron microscope (TEM) for sequencing. See, e.g., Perkel 2011.

### IV. Systems and Computer Readable Media

Another aspect provided herein relates to systems (and non-transitory computer readable media for causing computer systems), e.g., to perform a method for determining a given state and/or condition of a cell or a tissue sample, and/or to perform the methods of various aspects described herein, based on determining presence and/or absence and/or level(s) of one or more miRNA sequence described herein. In some embodiments, the systems and computer readable media described herein can be used to diagnose and/or prognose a condition or a stage of the condition in a subject.

In some embodiments, the device or computer system can further comprise a non-transitory computer-readable storage medium storing the one or more programs for execution by the one or more processors of the device or computer system.

In some embodiments, the device or computer system can further comprise one or more input devices, which can be configured to send or receive information to or from any one from the group consisting of: an external device (not shown), the one or more processors, the memory, the non-transitory computer-readable storage medium, and one or more output devices.

In some embodiments, the device or computer system can further comprise one or more output devices, which can be configured to send or receive information to or from any one from the group consisting of: an external device (not shown), the one or more processors, the memory, and the non-transitory computer-readable storage medium.

In some embodiments, the device or computer system for determining a given state and/or condition of a cell or tissue sample (biological sample) comprises one or more processors and memory to store one or more programs, the one or more programs comprising instructions for: (i) measuring in a biological sample the level(s) of one or more miRNA sequences; (ii) comparing the measured level(s) of one or more miRNA sequences to that of at least one or more reference samples to determine any significant alteration or difference in the level(s) of the miRNAs between the biological sample and the reference sample(s); and (iii) displaying a content based in part on the data output from (ii), wherein the content comprises a signal indicative of a subject having, or being at risk of developing, or being at a given stage of a disease or disorder, or a signal indicative of no sign of the disease or disorder.

A device or a system (e.g., a computer system) for obtaining data from at least one biological sample obtained from one or more subjects is disclosed. Accordingly, a system for analyzing a biological sample is provided herein. The system can be used to diagnose or prognose a condition or state of a condition in a subject. The system comprises: (a) a determination module configured to receive a biological sample and to determine sequence information, wherein the sequence information comprises a sequence of at least one or more miRNA molecules from the biological sample; (b) a storage device configured to store sequence information from the determination module; (c) a comparison module adapted to compare the sequence information stored on the storage device with reference data, and to provide a comparison result, wherein the comparison result identifies the presence or absence or level(s) of the miRNA molecule(s), wherein a discrepancy in an expression level of the miRNA molecule(s) from the reference data is indicative of the biological sample having an increased likelihood of having or being at a cellular or tissue state different from a state represented by the reference data; and (d) a display module for displaying a content based in part on the comparison result for the user, wherein the content is a signal indicative of a subject having, or being at risk of developing, or being at a given stage of a disease or disorder, or a signal indicative of lacking a disease or disorder.

A tangible and non-transitory (e.g., no transitory forms of signal transmission) computer readable medium having computer readable instructions recorded thereon to define software modules for implementing a method on a computer is also provided herein. In some embodiments, the software modules can include a comparison module and a display module for implementing a method on a computer. In some embodiments, the computer-readable medium stores one or more programs for determining a given state of a condition of a cell or tissue (a biological sample). In some embodiments, the computer-readable medium stores one or more programs for determining a condition or state of a condition of a subject. The one or more programs for execution by one or more processors of a computer system comprises (a) instructions for comparing the measured miRNA data (from a biological sample) stored on a storage device with reference data to provide a comparison result, wherein the comparison result the comparison result identifies the presence or absence (or difference in levels) of the miRNA molecule(s), wherein a discrepancy in level(s) of the miRNA molecule(s) from the reference data is indicative of the biological sample having an increased likelihood of having or being at a cellular or tissue state different from a state represented by the reference data; and (b) instructions for displaying a content based in part on the comparison result for the user, wherein the content is a signal indicative of a subject having, or being at risk of developing, or being at a given stage of a disease or disorder, or a signal indicative of lacking a disease or disorder.

Embodiments provided herein have been described through functional modules, which are defined by computer executable instructions recorded on computer readable media and which cause a computer to perform method steps when executed. The modules have been segregated by function for the sake of clarity. However, it should be understood that the modules need not correspond to discrete blocks of code and the described functions can be carried out by the execution of various code portions stored on various media and executed at various times. Furthermore, it should be appreciated that the modules may perform other functions, thus the modules are not limited to having any particular functions or set of functions.

Computing devices typically include a variety of media, which can include computer-readable storage media and/or communications media, in which these two terms are used herein differently from one another as follows. Computer-readable storage media or computer readable media can be any available tangible media (e.g., tangible storage media) that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM (random access memory), ROM (read only memory), EEPROM (erasable programmable read only memory), flash memory or other memory technology, CD-ROM (compact disc read only memory), DVD (digital versatile disk) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In some embodiments, the computer readable storage media can include the “cloud” system, in which a user can store data on a remote server, and later access the data or perform further analysis of the data from the remote server.

Computer-readable data embodied on one or more computer-readable media, or computer readable medium, can define instructions, for example, as part of one or more programs, that, as a result of being executed by a computer, instruct the computer to perform one or more of the functions described herein (e.g., in relation to a system, or computer readable medium), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of system 10, or computer readable medium described herein, can be distributed across one or more of such components, and may be in transition there between.

Computer executable instructions can be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Baxevanis Bioinformatics: A Practical Guide for Analysis of Gene and Proteins (Wiley & Sons, Inc., 2nd ed., 2001).

The functional modules of certain embodiments provided herein include a determination module, a storage device, a comparison module and a display module. The functional modules can be executed on one, or multiple, computers, or by using one, or multiple, computer networks. The determination module has computer executable instructions to provide sequence information in computer readable form. As used herein, “sequence information” refers to any nucleotide sequence, including but not limited to full-length sequence, partial sequence, or mutated sequences. Moreover, information “related to” the sequence information includes detection of the presence or absence of a miRNA sequence, determination of the expression level of a miRNA sequence in a biological sample, and the like. In some embodiments, the sequence information can include sequences of any miRNA molecules present in a biological sample. In some embodiments, the sequence information can include sequences of miRNA molecules. In other embodiments, the sequence information can include sequences of miRNA molecules described herein, miRNA molecules, piRNA molecules, mRNA molecules, or any combinations thereof. In some embodiments, the sequence information can include sequences of miRNA molecules present in a biological sample, and a genomic sequence of one or more protein-coding genes.

As an example, determination modules for determining sequence information can include known systems for automated sequence analysis, including but not limited to, Hitachi FMBIO® and Hitachi FMBIO® II Fluorescent Scanners (available from Hitachi Genetic Systems, Alameda, Calif.); Spectrumedix® SCE 9610 Fully Automated 96-Capillary Electrophoresis Genetic Analysis Systems (available from SpectruMedix LLC, State College, Pa.); ABI PRISM® 377 DNA Sequencer, ABI® 373 DNA Sequencer, ABI PRISM® 310 Genetic Analyzer, ABI PRISM® 3100 Genetic Analyzer, and ABI PRISM® 3700 DNA Analyzer (available from Applied Biosystems, Foster City, Calif.); Molecular Dynamics Fluorlmager™ 575, SI Fluorescent Scanners, and Molecular Dynamics Fluorlmager™ 595 Fluorescent Scanners (available from Amersham Biosciences UK Limited, Little Chalfont, Buckinghamshire, England); GenomyxSC™ DNA Sequencing System (available from Genomyx Corporation (Foster City, Calif.); and Pharmacia ALF™ DNA Sequencer and Pharmacia ALFexpress™ (available from Amersham Biosciences UK Limited, Little Chalfont, Buckinghamshire, England); any next- or higher-generation sequencing instruments such as, but not limited to, GF GLX Titanium, GS Junior (available from 454 Life Sciences, part of Roche Diagnostic Corporation, Branford, Conn.); HiSeq 2000, Genome Analyzer ILX, Genome Analyzer IIE, iScan SQ (available from Illumina, San Diego, Calif.); ABI SOLiD™ system (e.g., SOLiD4 platform available from Life Technologies, Applied Biosystems, Carlsbad, Calif.); HeliScope™ Single Molecule Sequencer (available from Helicos Biosciences Corporation, Cambridge, Mass.); and PACBIO RS (available from Pacific Biosciences, Menlo Park, Calif.).

Alternative methods for determining sequence information, i.e., determination modules 40, include systems for nucleic acid analysis. For example, mass spectrometry systems including Matrix Assisted Laser Desorption Ionization-Time of Flight (MALDI-TOF) systems and SELDI-TOF-MS ProteinChip array profiling systems; systems for analyzing gene expression data (see, for example, published U.S. Patent Pub. No. U.S. 2003/0194711); systems for array based expression analysis: e.g., HT array systems and cartridge array systems such as GeneChip® AutoLoader, Complete GeneChip® Instrument System, GeneChip® Fluidics Station 450, GeneChip® Hybridization Oven 645, GeneChip® QC Toolbox Software Kit, GeneChip® Scanner 3000 7G plus Targeted Genotyping System, GeneChip® Scanner 3000 7G Whole-Genome Association System, GeneTitan™ Instrument, and GeneChip® Array Station (each available from Affymetrix, Santa Clara, Calif.); Densitometers (e.g., X-Rite-508-Spectro Densitometer® (available from RP Imaging™, Tucson, Ariz.), The HYRYS™ 2 HIT densitometer (available from Sebia Electrophoresis, Norcross, Ga.); automated Fluorescence in situ hybridization systems (see for example. U.S. Pat. No. 6,136,540); 2D gel imaging systems coupled with 2-D imaging software; microplate readers; Fluorescence activated cell sorters (FACS) (e.g., Flow Cytometer FACSVantage SE, (available from Becton Dickinson, Franklin Lakes, N.J.); and radio isotope analyzers (e.g., scintillation counters).

The sequence information determined in the determination module can be read by the storage device. As used herein the “storage device” is intended to include any suitable computing or processing apparatus or other device configured or adapted for storing data or information. Examples of electronic apparatus suitable for use with various embodiments described herein can include stand-alone computing apparatus, data telecommunications networks, including local area networks (LAN), wide area networks (WAN), Internet, Intranet, and Extranet, and local and distributed computer processing systems. Storage devices 30 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon sequence information or expression level information. Such information may be provided in digital form that can be transmitted and read electronically, e.g., via the Internet, on diskette, via USB (universal serial bus) or via any other suitable mode of communication.

As used herein, “expression level information” refers to any nucleotide expression level information, including but not limited to full-length nucleotide sequences, partial nucleotide sequences, or mutated sequences. Moreover, information “related to” the expression level information includes detection of the presence or absence of a sequence (e.g., presence or absence of a nucleotide sequence), determination of the concentration of a sequence in the sample (e.g., nucleotide (RNA or DNA) expression levels), and the like.

As used herein, “stored” refers to a process for encoding information on the storage device. Those skilled in the art can readily adopt any of the presently known methods for recording information on known media to generate manufactures comprising the sequence information or expression level information.

A variety of software programs and formats can be used to store the sequence information or expression level information on the storage device. Any number of data processor structuring formats (e.g., text file or database) can be employed to obtain or create a medium having recorded thereon the sequence information or expression level information.

By providing sequence information or expression level information in computer-readable form, one can use the sequence information or expression level information in readable form in the comparison module 80 to compare a specific sequence or expression profile with the reference data within the storage device 30. In some embodiments, the comparison module 80 can also include bioinformatics analysis tools for next-generation sequencing data (e.g., short-read sequence data). Examples of bioinformatics analysis tools for next-generation sequencing (NGS) data can include any commercial NGS analysis packages that are compatible with the sequenced reads obtained from the NGS instrument. The NGS analysis package can include a sequence mapping tool for mapping sequences (e.g., short-read sequences) to a reference genome, sequence assembly tool for de novo assembly of overlapping reads to form contiguous nucleic acid sequence, a genome browser, and any combinations thereof. Examples of short-read alignment tools for mapping miRNA sequences to a reference genome can include, without limitations, Bfast, BioScope, Bowtie, Burrows-Wheeler Aligner (BWA), CLC bio, CloudBurst, Eland/Eland2, Exonerate, GenomeMapper, GnuMap, Karma, MAQ, MOM, Mosaik, MrFAST/MrsFAST, NovoAlign, PASS, PerM, RazerS, RMAP, SSAHA2, Segemehl, SeqMap, SHRiMP, Slider/Sliderll, SOAP/SOAP2, Srprism, Stampy, vmatch, ZOOM and any art-recognized alignment tools that can be used to align short-read sequences to a reference genome. In one embodiment, Burrows-Wheeler Aligner (BWA) can be used to map miRNA sequences to a reference genome (e.g., a human genome). Examples of sequence assembly tools include, but are not limited to, ABySS, ALLPATHS, Edena, Euler-SR, SHARCGS, SHARP, SSAKE, Velvet and any other art-recognized assembly tools. Different genome browsers can be used to visualize genomic maps, e.g., generated after sequence alignment to a reference genome.

In one embodiment, the comparison module uses sequence information alignment programs such as BLAST (Basic Local Alignment Search Tool) or FAST (using the Smith-Waterman algorithm) that may be employed individually or in combination. These algorithms determine the alignment between similar regions of sequences and a percent identity between sequences.

In some embodiments, the comparison module can include a pattern recognition pattern that can be pre-trained with different reference data sets such as data sets comprising profiles of miRNA sequences obtained from different state of a tissue (e.g., normal data set vs. diseased or abnormal data set; or data sets corresponding to different stages of a disease or disorder and a normal data set).

Accordingly, in some embodiments, the comparison module can compare a profile of miRNA sequences of a biological sample determined by the determination module 40 to reference data stored on the storage device, and classify the biological sample into a specific state (e.g., normal, diseased or abnormal, and/or a given stage of a disease or disorder). For example, comparison programs can be used to compare an expression level of a miRNA sequence in a biological sample to a reference data expression level (e.g., sequence data from a control/reference sample described herein) and/or profiles of miRNA sequences in a biological sample to reference data expression profiles (e.g., sequence data from a control/reference sample described herein). The comparison made in computer-readable form provides a computer readable comparison result, which can be processed by a variety of means. Content based on the comparison result can be retrieved from the comparison module to indicate a given state of a cell or a tissue, and/or whether a subject has, or is at risk of developing of a disease or disorder, or a given state of the disease or disorder.

In one embodiment, the reference data stored in the storage device to be read by the comparison module 80 is sequence information data obtained from a reference sample described herein or a control biological sample of the same type as the biological sample to be tested.

Alternatively, the reference data are a database, e.g., a collection of sequence information data obtained from a plurality of reference samples described herein and control biological samples of the same type as the biological sample to be tested. For example, reference data can include profiles of miRNA sequences that are indicative of a given state of a cell or tissue and/or a disease or disorder of interest or a given state of the disease or disorder. In one embodiment, the reference data can include sequence information of miRNA sequences and/or profiles of miRNA sequences that are indicative of a disease or disorder of interest, e.g., a disease or disorder afflicting a tissue, and or different stages of a disease or disorder of interest, e.g., different stages of cancer. By way of example only, reference data stored in a system for diagnosing and/or prognosing breast cancer can include, but not limited to, (a) profile(s) of miRNA sequences obtained from one or a group of normal subjects, (b) profile(s) of miRNA sequences obtained from one or a group of subjects having a given stage of breast cancer (e.g., DCIS, lobular carcinoma in situ, INV, etc.); (c) profile(s) of miRNA obtained from a normal tissue of the test subject, profile(s) of miRNA sequences obtained from a diseased or abnormal tissue of the test subject that was previously diagnosed, and any combinations thereof.

In one embodiment, the reference data are electronically or digitally recorded and annotated from databases including, but not limited to GenBank (NCBI) protein and DNA databases such as genome, ESTs, SNPS, Traces, Celara, Ventor Reads, Watson reads, HGTS, and the like; Swiss Institute of Bioinformatics databases, such as ENZYME, PROSITE, SWISS-2DPAGE, Swiss-Prot and TrEMBL databases; the Melanie software package or the ExPASy web server, and the like; the SWISS-MODEL, Swiss-Shop and other network-based computational tools; the Comprehensive Microbial Resource database (available from The Institute of Genomic Research). The resulting information can be stored in a relational database that may be employed to determine homologies between the reference data or genes or proteins within and among genomes.

The “comparison module” can use a variety of available software programs and formats for the comparison operative to compare sequence information determined in the determination module to reference data. In one embodiment, the comparison module is configured to use pattern recognition techniques to compare sequence information from one or more entries to one or more reference data patterns. The comparison module can be configured using existing commercially-available or freely-available software for comparing patterns, and may be optimized for particular data comparisons that are conducted. The comparison module provides computer readable information related to the sequence information that can include, for example, detection of the presence or absence of a miRNA sequence; determination of the concentration of a miRNA sequence in the sample, or determination of an expression profile.

The comparison module, or any other module described herein, may include an operating system (e.g., UNIX) on which runs a relational database management system, a world-wide-web application, and a world-wide-web server. The world-wide-web application includes the executable code necessary for generation of database language statements (e.g., Structured Query Language (SQL) statements). Generally, the executables will include embedded SQL statements. In addition, the World Wide Web application may include a configuration file, which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the world-wide-web server supports a TCP/IP protocol. Local networks such as this are sometimes referred to as “Intranets.” An advantage of such Intranets is that they allow easy communication with public domain databases residing on the world-wide-web (e.g., the GenBank or Swiss Pro world-wide-web site). Thus, in a particular preferred embodiment provided herein, users can directly access data (via hypertext links for example) residing on Internet databases using a HTML interface provided by web browsers and web servers. In one embodiment, users can access data residing on Cloud storage.

Various algorithms or software packages are available which are useful for comparing and analyzing sequence information and/or expression data determined in the determination module 40. For example, various software packages for next-generation sequencing (NGS) analysis are available in the commercial and/or public domains. Exemplary software packages for NGS analysis can include, without limitations, sequence alignment tools as discussed above; de novo alignment and/or assembly tools as discussed above; integrated solutions, such as CLCbio Genomics Workbench, Galaxy, Genomatix, JMP Genomics, NExtGENE, SeqMan Genome Analyzer, SHORE, SlimSearch; genome browser (including alignment viewer and/or assembly database) such as EagleView, LookSeq, MapView, Sequence Assembly Manager, STADEN, XMatchView; software packages for transciptomics such as ERANGE, S-Mo.R-Se, MapNext, QPalma, RSAT, TopHat; or any combinations thereof.

In some embodiments, when the sequence information is determined by microarray-based methods, various software packages for microarray analysis can be used, e.g., but not limited to, GeneChip® Sequence Analysis Software (GSEQ), GeneChip® Targeted Genotyping Analysis Software (GTGS) and Expression Console™ Software. Accordingly, depending on methods used to produce sequence information in the determination module, various sequence analysis software can be used.

In one embodiment described herein, pattern comparison software is used to compare an expression profile of miRNA sequences to a reference data for determining a given state of a cell or tissue, or whether the expression profiled obtained from a test subject is indicative of a disease or disorder, or a given state of a disease or disorder.

The comparison module provides computer readable comparison result that can be processed in computer readable form by predefined criteria, or criteria defined by a user, to provide a content based in part on the comparison result that may be stored and output as requested by a user using a display module. The display module enables display of a content based in part on the comparison result for the user, wherein the content is a signal indicative of a subject having, or being at risk of developing or being at a given stage of a disease or disorder, or a signal indicative of the subject having no risk of the disease or disorder. Such signal, can be for example, a display of content indicative of the presence or absence of increased risk for a disease or disorder, or a given state of a disease or disorder on a computer monitor, a printed page of content indicating the presence or absence of increased risk for a given state of a disease or disorder from a printer, or a light or sound indicative of the presence or absence of increased risk for a given state of a disease or disorder.

The content based on the comparison result can include an expression profile of one or more miRNA sequences determined from the test subject. In one embodiment, the content based on the comparison result can include a comparison of the miRNA expression profile between the test subject and one or more reference samples described herein. In one embodiment, the content based on the comparison result is merely a signal indicative of the presence or absence of an increased risk of a given state of a disease or disorder.

In one embodiment provided herein, the content based on the comparison result is displayed a on a computer monitor. In one embodiment, the content based on the comparison result is displayed through printable media. The display module can be any suitable device configured to receive from a computer and display computer readable information to a user. Non-limiting examples include, for example, general-purpose computers such as those based on INTEL® processor, QUALCOMM® processors, Sun Microsystems processors, Hewlett-Packard processors, any of a variety of processors available from Advanced Micro Devices (AMD) of Sunnyvale, Calif., or any other type of processors (including mobile processors), visual display devices such as tablet computers, flat panel displays, cathode ray tubes and the like, as well as computer printers of various types.

In one embodiment, a world-wide-web browser is used for providing a user interface for display of the content based on the comparison result. It should be understood that other modules described herein can be adapted to have a web browser interface. Through the web browser, a user may construct requests for retrieving data from the comparison module. Thus, the user will typically point and click to user interface elements such as buttons, pull down menus, scroll bars and the like conventionally employed in graphical user interfaces. The requests formulated with the user's web browser are transmitted to a web application which formats them to produce a query that can be employed to extract the pertinent information related to the sequence information, e.g., but not limited to, display of nucleotide (RNA or DNA) expression levels; or display of information based thereon. In one embodiment, the sequence information of the reference sample data is also displayed.

In one embodiment, the display module displays the comparison result based on sequence information and whether the comparison result is indicative of a disease or disorder, or a given stage of a disease or disorder. For example, in the case of diagnosis of breast cancer, the display module can display the comparison result based on determined sequence information and whether the comparison results is indicative of breast cancer, or a particular stage of breast cancer (e.g., ductal carcinoma in situ, lobular carcinoma in situ, invasive, etc.).

In one embodiment, the content based on the comparison result that is displayed is a signal (e.g., positive or negative signal) indicative of the presence or absence of an increased risk for a disease or disorder, or a given stage of the disease or disorder, thus only a positive or negative indication may be displayed.

In any embodiments, the comparison module can be executed by a computer implemented software as discussed earlier. In such embodiments, a result from the comparison module can be displayed on an electronic display. The result can be displayed by graphs, numbers, characters or words. In additional embodiments, the results from the comparison module can be transmitted from one location to at least one other location. For example, the comparison results can be transmitted via any electronic media, e.g., internet, fax, phone, a “cloud” system, and any combinations thereof. Using the “cloud” system, users can store and access personal files and data or perform further analysis on a remote server rather than physically carrying around a storage medium such as a DVD or thumb drive.

Each of the above identified modules or programs corresponds to a set of instructions for performing a function described above. These modules and programs (i.e., sets of instructions) need not be implemented as separate software programs, procedures or modules, and thus various subsets of these modules may be combined or otherwise re-arranged in various embodiments. In some embodiments, memory may store a subset of the modules and data structures identified above. Furthermore, memory may store additional modules and data structures not described above.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described herein can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

What has been described above includes examples of the embodiments of the present disclosure. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples arc described herein for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described herein. Furthermore, not all illustrated acts may be required to implement the methodologies in accordance with the disclosed subject matter. In addition, those skilled in the art will understand and appreciate that the methodologies could alternatively be represented as a series of interrelated states via a state diagram or events. Additionally, it should be appreciated that the methodologies disclosed in this specification are capable of being stored on an article of manufacture to facilitate transporting and transferring such methodologies to computing devices. The term article of manufacture, as used herein, is intended to encompass a computer program accessible from any computer-readable device or storage media.

Provided herein therefore relates to systems (and computer readable medium for causing computer systems) to perform methods for determining a given stage of a cell or a tissue, and/or whether a subject has, or is at risk of developing, or is at a given stage of a disease, e.g., cancer, or disorder, based on expression profiles of miRNA sequences.

System and computer readable medium, are merely an illustrative embodiments provided herein for performing methods of determining whether an individual has a specific disease or disorder or a pre-disposition, for a specific disease or disorder based on expression profiles or sequence information, and are not intended to limit the scope described herein. Variations of system, and computer readable medium, are possible and are intended to fall within the scope described herein.

The modules of the machine, or used in the computer readable medium, may assume numerous configurations. For example, function may be provided on a single machine or distributed over multiple machines.

### V. Reference Sample(s) or Reference Data

As used herein, a reference sample can include a normal or negative control, alternatively a disease (or disorder) or positive control, against which biological samples can be compared. Therefore, it can be determined whether the biological sample to be evaluated for a specific disease or disorder, or a stage of a disease or disorder, has measurable difference or substantially no difference, as compared to a reference sample. A normal or healthy sample or tissue refers to a sample or tissue that does not have a disease or disorder to be evaluated.

The reference sample can be obtained from the patient to be diagnosed or prognosed, or from a different subject, who is preferably of same age and/or race.

In one embodiment, the reference sample can be obtained from the same patient at the same time that the biological sample is taken. In one embodiment, the reference sample can be taken from a normal and/or healthy tissue of the same patient. In one embodiment, the reference sample can be taken from a normal and/or healthy tissue, for example tissue taken adjacent to the cancer, such as within 1 or 2 cm diameter from the leading front of the tumor. Alternatively, the reference sample can be taken from an equivalent position in the subject's body, for example in the case of breast cancer, a reference sample can be taken from any area of the breast which is not cancerous. In another embodiment, the reference sample can be a disease or abnormal sample taken previously from the same patient, against which a new biological sample can be compared to provide an evaluation of the therapeutic treatment efficacy.

In one embodiment, the reference sample can be a sample taken previously, e.g., a sample of the same or a different cancer/tumor, the comparison of which can, for example, provide characterization of the source of the new tumor, and/or progression or development of an existing cancer, such as before, during or after therapeutic treatment. For example, the reference sample can be obtained from a different patient, e.g., it can be a control sample, or a collection of control samples, representing different stages or different types of diseases or disorders. In one embodiment, the reference sample can be a control sample or a collection of control samples, representing different stages of a specific cancer (e.g., cancer staging samples) or different types of cancer, for example those listed herein (i.e., cancer reference samples). Comparison of the biological sample data with data obtained from such cancer staging or cancer reference samples can, for example, allow for the characterization of the assessed cancer to a specific stage and/or type of cancer.

Depending on various applications, the reference sample can comprise a sample derived from a tissue type that is same as or different from the tissue type of a biological sample. In some embodiments wherein the tissue origin of a biological sample is unknown, the level(s) and/or profile of the miRNA present in the biological sample can be compared to a set of reference samples of different tissue origins in order to identify the tissue origin of the biological sample. In some embodiments where the tissue origin of a biological sample is known or believed to be known (e.g., while a tissue biopsy is known to be collected from a lung tissue, the sample can comprise cells originated from breast due to metastasis), the reference sample(s) can comprise a sample of the same tissue type as the biological sample, and/or a sample of a different tissue type from the biological sample. By comparison between the biological sample and reference sample(s), tissue origin of the biological sample can be validated and/or identified.

As used herein, the term “reference data” refers to data obtained from a reference sample as described herein, or a collection of reference samples as described herein.

### VI. Biological Samples and Preparation Thereof

A “biological sample” subjected to analysis using the methods, assays and systems described herein generally refers to a sample taken or isolated from a subject or a biological organism. In some embodiments, the biological sample contains one or more cells, e.g., tissue culture mammalian cells, cell lysate, a tissue sample from a subject, a homogenate of a tissue sample from a subject or a fluid sample from a subject. Exemplary biological samples include, but are not limited to, blood (including whole blood, serum, cord blood, and plasma), sputum, urine, spinal fluid, pleural fluid, nipple aspirates, lymph fluid, the external sections of the skin, respiratory, intestinal, and genitourinary tracts, tears, saliva, milk, feces, sperm, cells or cell cultures, serum, leukocyte fractions, smears, tissue samples of all kinds, embryos, etc. The term also includes both a mixture of the above-mentioned samples such as whole human blood containing a cell. The term “biological sample” also includes untreated or pretreated (or pre-processed) biological samples.

A “biological sample” can contain at least one cell or a plurality of cells from a subject. In some embodiments, the biological sample can contain one or more somatic cells from a subject. In other embodiments, the biological sample can contain one or more germ cells from a subject. In other embodiments, the biological sample can contain one or more stem cells from a subject.

In one embodiment, the biological sample can contain one or more cells from a subject's biological fluid sample. Examples of biological fluids include, but are not limited to, saliva, bone marrow, blood, serum, plasma, urine, sputum, cerebrospinal fluid, an aspirate, tears, and any combinations thereof. The biological sample can contain one or more circulating tumor cells from a subject's blood (including whole blood, serum, cord blood, and plasma). In some embodiments, the biological sample can contain at least one type of blood cells (e.g., red blood cells, white blood cells, platelets).

In one embodiment, the biological sample can contain one or more cells derived from any tissue of a subject, e.g., a tissue suspected of being at risk of, or being afflicted with a given stage of a disease or a disorder. Non-limiting examples of a tissue can include, but are not limited to, breast, pancreas, blood, prostate, colon, lung, skin, brain, ovary, kidney, oral cavity, throat, liver, and any combinations thereof. In some embodiments, the tissue can be obtained from a resection, biopsy, or core needle biopsy. In addition, fine needle aspirate samples can be used. Samples can be either paraffin-embedded or frozen tissue.

The biological sample can be obtained by removing a sample of cells from a subject, but can also be accomplished by using previously isolated cells (e.g., isolated by another person). In addition, the biological sample can be freshly collected or a previously collected sample.

In some embodiments, the biological sample is a frozen biological sample, e.g., a frozen tissue or fluid sample such as urine, blood, serum or plasma. The frozen sample can be thawed before employing methods, assays and systems described herein. After thawing, a frozen sample can be centrifuged before being subjected to methods, assays and systems described herein.

In some embodiments, a biological sample can be a nucleic acid product derived from a tissue (e.g., fresh/frozen and paraffin-embedded) or a fluid sample (e.g., blood) of a subject or cultured cells. The nucleic acid product can include DNA, RNA, mRNA, miRNA, piRNA, siRNA, snRNA, miRNA molecules described herein, and any combinations thereof. In some embodiments, the nucleic acid product can comprise one or more miRNA molecules described herein.

In some embodiments, a biological sample can include RNA isolated from a tissue (e.g., fresh or frozen or paraffin-embedded) or a fluid sample (e.g., blood) of a subject or cultured cells. Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al., PCR: Clinical Diagnostics and Research, Springer (1994)).

In one embodiment, a biological sample can include RNA isolated from a tissue (e.g., fresh or frozen or paraffin-embedded) by any known methods in the art. When the RNA sample is deemed to be of good quality (according to one of skill in the art), the sample can be subjected to further treatment, following recommended instructions as provided by various commercial RNA preparation kits available for RNA sequencing (e.g., the kits from Life Technologies). Depending on the length of RNA molecules of interest, in some embodiments, the RNA sample can be subjected to miRNA sequencing. In other embodiments, the RNA sample can be subjected to long RNA sequencing.

In some embodiments, a biological sample can be an enriched RNA fraction derived from a tissue (e.g., fresh/frozen and paraffin-embedded) or a fluid sample (e.g., blood) of a subject or cultured cells, e.g., an RNA fraction enriched for non-coding RNAs. This can be achieved, for example, by removing mRNAs by use of affinity purification, e.g., using an oligodT column or any other art-recognized methods such as using commercial small RNA isolation kits.

In some embodiments, a biological sample can be a nucleic acid product or an RNA fraction amplified after polymerase chain reaction (PCR) or after reverse transcription-PCR. The nucleic acid product can include DNA (e.g., cDNA), RNA and mRNA and can be isolated from a particular biological sample using any of a number of procedures, which are well known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russell, Cold Spring Harbor Laboratory Press, 2001.

In some embodiments, the biological sample can be treated with a chemical and/or biological reagent. Chemical and/or biological reagents can be employed to protect and/or maintain the stability of the sample, including biomolecules (e.g., nucleic acids) therein, during processing. One exemplary reagent is an RNase inhibitor or RNA stabilizer, which is generally used to protect or maintain the stability of RNA during processing. In addition, or alternatively, chemical and/or biological reagents can be employed to release nucleic acid (e.g., miRNA molecules) from the biological sample.

The skilled artisan is well aware of methods and processes appropriate for pre-processing of biological samples required for determination of nucleic acid including miRNA molecules as described herein.

In some embodiments, a biological sample used for determining the level of one or more miRNAs can be a sample containing circulating miRNAs, e.g., extracellular miRNAs. Extracellular miRNAs freely circulate in a wide range of biological material, including bodily fluids, such as fluids from the circulatory system, e.g., a blood sample or a lymph sample, or from another bodily fluid such as cerebrospinal fluid (CSF), urine or saliva. Accordingly, in some embodiments, the biological sample used for determining the level of one or more miRNAs can be a bodily fluid, for example, blood, fractions thereof, serum, plasma, urine, saliva, tears, sweat, semen, vaginal secretions, lymph, bronchial secretions, CSF, etc. In some embodiments, the sample is a sample that is obtained non-invasively.

Circulating miRNAs include miRNAs in cells (cellular miRNA), extracellular miRNAs in microvesicles (microvesicle-associated miRNA), and extracellular miRNAs that are not associated with cells or microvesicles (extracellular, non-vesicular miRNA).

The biological sample is generally obtained from a subject who has or is suspected of having a disease or disorder, e.g., a condition afflicting a tissue, or who is suspected of having a risk of developing a disease or disorder, e.g., a condition afflicting a tissue. In some embodiments, the biological sample can be obtained from a subject who has or is suspected of having cancer, or who is suspected of having a risk of developing cancer. By way of example only, in one embodiment, the biological sample can be obtained from a subject who has or is suspected of having breast cancer, or who is suspected of having a risk of breast cancer. In another embodiment, the biological sample can be obtained from a subject who has or is suspected of having pancreatic cancer, or who is suspected of having a risk of pancreatic cancer.

In some embodiments, the biological sample can be obtained from a subject who is being treated for the disease or disorder, e.g., but not limited to, cancer such as breast cancer or pancreatic cancer. In other embodiments, the biological sample can be obtained from a subject whose previously-treated disease or disorder, e.g., but not limited to, cancer such as breast cancer or pancreatic cancer, is in remission. In other embodiments, the biological sample can be obtained from a subject who has a recurrence of a previously-treated disease or disorder, e.g., but not limited to, cancer such as breast cancer or pancreatic cancer.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, and avian species, e.g., chicken, emu, ostrich. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female. The term “patient” and “subject” does not denote a particular age. Thus, any mammalian subjects from adult to newborn subjects, as well as fetuses, are intended to be covered.

In one embodiment, the subject or patient is a mammal. The mammal can be a human, non-human primate, mouse, rat, dog, cat, horse, or cow, but are not limited to these examples. In one embodiment, the subject is a human being. In another embodiment, the subject can be a domesticated animal and/or pet. In some embodiments, the subject is a human.

### VII. Pharmaceutical Compositions and Administration Thereof

In some embodiments, a subject in need thereof, e.g., whose level(s) of one or more miRNAs disclosed herein in a biological sample are determined to be over-expressed or under-expressed, can be administered with a miRNA-related therapeutic. In some embodiments, the miRNA-related therapeutic is an antagonist against a target miRNA that inhibit the function of the target miRNA. As used herein, the term “antagonist” is used in the broadest sense, and includes any molecule that partially or fully blocks, inhibits, or neutralizes a biological activity of a microRNA disclosed herein. Suitable antagonist molecules specifically include, but are not limited to antisense oligonucleotides, small organic molecules, aptamers, etc. Methods for identifying antagonists of a microRNA can comprise contacting a target microRNA with a candidate antagonist molecule and measuring a detectable change in one or more biological activities normally associated with the microRNA. Accordingly, one aspect provides a pharmaceutical composition comprising an antagonist against one or more miRNAs disclosed herein.

In some embodiments, the miRNA-related therapeutic is a mimic of a target miRNA that induces or restores the function of the target miRNA. As used herein, the term “mimic” refers to a molecule (e.g., an oligonucleotide) that is capable of mimicking the activity of a miRNA molecule. In some embodiments, a miRNA mimic is a molecule (e.g., an oligonucleotide) that is capable of mimicking at least about 30% or above (including, e.g., at least about 40%, at least about 50%, at least about 60%, at least about 70%, at least about 80%, at least about 90%, at least about 95%, at least about 97%, at least about 99%, or up to 100%) of the activity of a miRNA molecule.

Accordingly, a pharmaceutical composition comprising a mimic of one or more miRNAs disclosed herein. In some embodiments, the pharmaceutical composition can comprise engineered transcripts that can “sponge” various combinations of the miRNAs described herein, or any combinations thereof.

In some embodiments, the miRNA mimics can be a double-stranded and/or blunt-ended oligonucleotide, which means the oligonucleotide is double-stranded throughout the molecule and/or blunt-ended on both ends. In some embodiments, the miRNA mimics can be a single-strand oligonucleotide. In some embodiments, the single-stranded oligonucleotide can comprise a hairpin structure. Methods of making miRNA mimics are known in the art, e.g., as described in the International Patent Publication No. WO 2012/106586, which is incorporated herein by reference.

In some embodiments where the miRNA antagonists or miRNA mimics comprise an oligonucleotide, the oligonucleotide can be modified. For example, in some embodiments, the backbone of the oligonucleotides described herein can be modified, e.g., to stabilize the oligonucleotides for in vivo delivery. For example, the modified backbone of the oligonucleotides can comprise morpholino subunits, phosphorothioate subunits, locked nucleic acid (LNA), peptide nucleic acid (PNC), hexitol nucleic acid (HNA), or any combinations thereof. See, e.g., Singh et al., 2010 for additional information about various examples of oligonucleotide backbones.

Additionally or alternatively, the oligonucleotides can be modified for a desired property, e.g., improved cell delivery, cell-targeting delivery, and/or enhanced stability. By way of example only, the oligonucleotides can be modified by conjugating it to a delivery-targeting moiety. As used herein, the term “delivery-targeting moiety” refers to a moiety that can facilitate binding of an oligonucleotide to the outer surface of a cell and/or uptake or endocytosis of the oligonucleotide into the cell. Any art-recognized delivery-targeting moiety, e.g., but not limited to, antibodies, peptides, proteins, aptamers, dendrimers, and molecules, can be used. In some embodiments, the delivery-targeting moiety is a cell surface receptor ligand. As used herein, a “cell surface receptor ligand” refers to a molecule that can bind to the outer surface of a cell. Exemplary, cell surface receptor ligand includes, for example, a cell surface receptor binding peptide, a cell surface receptor binding glycopeptide, a cell surface receptor binding protein, a cell surface receptor binding glycoprotein, a cell surface receptor binding organic compound, and a cell surface receptor binding drug.

Cell surface receptor ligands include, but are not limited to, cytokines, growth factors, hormones, antibodies, and angiogenic factors.

In some embodiments, the delivery-targeting moiety can also refer to a molecule that binds to or interacts with a target molecule. Typically the nature of the interaction or binding is noncovalent, e.g., by hydrogen, electrostatic, or van der Waals interactions, however, binding may also be covalent.

In some embodiments, the oligonucleotides described herein can be conjugated to a molecule in any methods known in the art. By way of example only, the oligonucleotides can be conjugated in the following methods: (i) peptide-oligonucleotide conjugates (POCs); (ii) carbohydrate-oligonucleotide conjugates (COCs); (iii) lipophilic oligonucleotide conjugates (LOCs); (iv) metal complex-oligonucleotide conjugates (MCOCs); (v) nanoparticle-oligonucleotide conjugates (NOCs); (vi) these can include bifunctional oligonucleotides, which have one domain that hybridizes to target RNA and a second domain that attracts activator proteins; (vii) peptide-conjugated oligonucleotides, which can be fused to a peptide domain consisting of cationic and hydrophobic residues that facilitates cell entry; (viii) cell penetrating peptide-oligonucleotide; and (ix) any combinations thereof. See, e.g., Singh et al., 2010 for additional information about some conjugation methods of oligonucleotides to a molecule.

In some embodiments, the oligonucleotides can be conjugated to a carrier molecule. In some embodiments, a carrier molecule can be a natural or synthetic polymer. For example, a carrier molecule can be cholesterol or an RNA aptamer and the like. A carrier molecule can be conjugated to the oligonucleotides at the 5′ and/or 3′ end, or at the 5′ and/or 3′ end of one of the strands, or at an internal nucleotide position.

In some embodiments, one or two strands of the oligonucleotides can be encoded by or delivered with a viral vector. A variety of viral vectors know in the art can be modified to express or carry an oligonucleotide into a target cell, for example herpes simplex virus-1 or lentiviral vectors have been used to enhance the delivery of miRNA.

In some embodiments, an oligonucleotide can be associated with a non-viral vector. Non-viral vectors can be coupled to targeting and delivery enhancing moieties, such as antibodies, various polymers (e.g., PEG), fusogenic peptides, linkers, cell penetrating peptides and the like. Non-viral vectors include, but are not limited to liposomes and lipoplexes, polymers and peptides, synthetic particles and the like. In some embodiments, a liposome or lipoplex has a neutral, negative or positive charge and can comprise cardolipin, anisamide-conjugated polyethylene glycol, diolcoyl phosphatidylcholine, or a variety of other neutral, anionic, or cationic lipids or lipid conjugates. miRNAs can be complexed to cationic polymers (e.g., polyethylenimine (PEI)), biodegradable cationic polysaccharide (e.g., chitosan), or cationic polypeptides (e.g., atelocollagen, poly lysine, and protamine).

In some embodiments, oligonucleotide delivery can be enhanced by targeting the oligonucleotide to a cell. Targeting moieties can be conjugated to a variety of delivery compositions and provide selective or specific binding to a target cell(s). Targeting moieties can include, but are not limited to moieties that bind to cell surface receptors, cell specific extracellular polypeptide, saccharides or lipids, and the like. For example, small molecules such as folate, peptides such as RGD containing peptides, and antibodies such as antibodies to epidermal growth factor receptor can be used to target specific cell types.

In some embodiments, oligonucleotide delivery can be enhanced by moieties that interact with cellular mechanisms and machinery, such as uptake and intracellular trafficking. In certain aspects cell penetrating peptides (CPPs) (e.g., TAT and MPG from HIV-1, penetratin, polyarginine can be coupled with an miRNA or a delivery vector to enhance delivery into a cell. Fusogenic peptides (e.g., endodomain derivatives of HIV-1 envelope (HGP) or influenza fusogenic peptide (diINF-7)) can also be used to enhance cellular delivery.

A variety of delivery systems such as cholesterol-miRNA, RNA aptamers-miRNA, adenoviral vector, lentiviral vector, stable nucleic acid lipid particle (SNALP), cardiolipin analog-based liposome, DSPE-polyethylene glycol-DOTAP-cholesterol liposome, hyaluronan-DPPE liposome, neutral DOPC liposome, atelocollagen, chitosan, polyethylenimine, poly-lysine, protamine, RGD-polyethylene glycol-polyethylenimine, HER-2 liposome with histidine-lysine peptide, HIV antibody-protamine, arginine, oligoarginine(9R) conjugated water soluble lipopolymer (WSLP), oligoarginine (15R), TAT-PAMAM, cholesterol-MPG-8, DOPE-cationic liposome, GALA peptide-PEG-MMP-2 cleavable peptide-DOPE and the like have been used to enhance the delivery of miRNA.

As described in detail below, the pharmaceutical compositions described herein can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; (9) nasally; or (10) intrathecally. Additionally, one or more REST E2- and/or E3-skipping modulating agents can be implanted into a patient or injected using a drug delivery system. See, for example, Urquhart, et al., 1984; Lewis, ed. “Controlled Release of Pesticides and Pharmaceuticals” (Plenum Press, New York, 1981); U.S. Pat. Nos. 3,773,919; and 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C 12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The amount of oligonucleotide which can be combined with a carrier material to produce a single dosage form will generally be that amount of the compound which produces a therapeutic effect. Generally out of one hundred percent, this amount will range from about 0.1% to 99% of oligonucleotide, preferably from about 5% to about 70%, most preferably from 10% to about 30%.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred. As used herein, the term ED denotes effective dose and is used in connection with animal models. The term EC denotes effective concentration and is used in connection with in vitro models.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized.

The therapeutically effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay.

The dosage can be determined by a physician and adjusted, as necessary, to suit observed effects of the treatment. For example, a therapeutic dose range for the miRNA mimics can be 0.01-5.0 mg of miRNA per kg of patient body weight (mg/kg).

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered every day or every third, fourth, fifth, or sixth day. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In some embodiments, the pharmaceutical composition further includes at least a second therapeutic agent (e.g., an agent other than a miRNA antagonist or mimic described herein).

Exemplary therapeutic agents that can formulated with a miRNA antagonist or mimic described herein include, but are not limited to, those found in Harrison's Principles of Internal Medicine, 17th Edition, 2008, McGraw-Hill N.Y., NY; Physicians' Desk Reference, 63rd Edition, 2008, Thomson Reuters, N.Y., N.Y.; Goodman & Gilman's The Pharmacological Basis of Therapeutics, 11th Edition, 2005, McGraw-Hill N.Y., NY; United States Pharmacopeia, The National Formulary, USP—32 NF-27, 2008, U.S. Pharmacopeia, Rockville, Md., the complete contents of all of which are incorporated herein by reference.

### VIII. Kits

Based on the identification of miRNA sequences associated with a condition or disease, one aspect described herein also provides for the design and preparation of detection reagents needed to identify or detect one or more novel miRNAs disclosed herein in a biological sample of a subject. Examples of detection reagents that can be used to identify the disclosed miRNA sequences in a biological sample can include a primer and a probe, wherein the probe can selectively hybridize the miRNA of interest.

Accordingly, provided herein include kits for determining whether a subject has, or is at risk of developing, or is at a given stage of condition afflicting a tissue of interest. In some embodiments, the kits can be used for monitoring the response of a subject to a therapeutic treatment. The kits can include at least one reagent specific for detecting for the presence or absence of at least one novel miRNAs described herein, and instructions for use.

In one embodiment, a kit can comprise an oligonucleotide array affixed with a plurality of oligonucleotide probes that interrogate no more than 100 novel miRNAs described herein (including no more than 75 miRNAs, no more than 50 miRNAs, no more than 25 miRNAs, no more than 20 miRNAs, no more than 15 miRNAs, no more than 10 miRNAs, no more than 5 miRNAs or less), wherein the miRNAs comprise at least two or any combinations of the novel miRNAs disclosed herein, and an optional container containing a detectable label (e.g., comprising a fluorescent molecule) to be conjugated to a nucleotide molecule derived from a test sample of a human subject; and at least one reagent. Examples of a reagent that can be included in the kit can include, without limitations, a restriction enzyme, a universal adaptor to be conjugated to a nucleotide molecule, a primer complementary to the universal adaptor, a wash agent, and any combinations thereof.

In some embodiments, the plurality of oligonucleotide probes affixed to an oligonucleotide array can interrogate about 2-100 miRNAs, e.g., about 3-50 miRNAs, about 3-25 miRNAs, about 3-10 miRNAs, or about 3-5 miRNAs, wherein the miRNAs comprise at least two or any combinations of the novel miRNAs disclosed herein.

Additional reagents included in the kit can vary with the selection of a sequencing method described herein.

### IX. Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this disclosure belongs.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

Other than in the operating examples, or where otherwise indicated, all numbers expressing quantities of ingredients or reaction conditions used herein should be understood as modified in all instances by the term “about.” The term “about” when used to describe the present disclosure, in connection with percentages means±1%.

In one respect, the present disclosure relates to the herein described compositions, methods, and respective component(s) thereof, as essential to the disclosure, yet open to the inclusion of unspecified elements, essential or not (“comprising”). In some embodiments, other elements to be included in the description of the composition, method or respective component thereof are limited to those that do not materially affect the basic and novel characteristic(s) of the disclosure (“consisting essentially of). This applies equally to steps within a described method as well as compositions and components therein. In other embodiments, the disclosures, compositions, methods, and respective components thereof, described herein are intended to be exclusive of any element not deemed an essential element to the component, composition or method (“consisting of).

The term “statistically significant” or “significantly” or “significant” refers to statistical significance and generally means a one standard deviation (1SD) above or below a reference level. Alternatively, statistical significance can be measured by means of a “false discovery rate” threshold, e.g., 0.05. The term refers to statistical evidence that there is a difference. It is defined as the probability of making a decision to reject the null hypothesis when the null hypothesis is actually true. The decision is often made using the p-value. Alternatively, the decision is made using the estimated false discovery rate.

The term “deep sequencing” as used herein generally refers to next- or higher-generation sequencing known to a skilled artisan.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, a uracil “U” or a C). The term “nucleic acid” encompasses the terms “oligonucleotide” and “polynucleotide,” each as a subgenus of the term “nucleic acid.” The term “oligonucleotide” refers to a molecule of between about 2 and about 100 nucleobases in length. The term “polynucleotide” refers to at least one molecule of greater than about 100 nucleobases in length.

The term “non-coding” refers to sequences of nucleic acid molecules that cannot be translated in a sequence-specific manner to produce into a particular polypeptide or peptide. In some embodiments, the term “non-coding” in reference to RNA can refer to a RNA sequence that is not translated in a sequence-specific manner to produce a particular polypeptide or peptide. In some embodiments, a non-coding RNA can comprise a sequence corresponding to a fragment of a protein-coding region, but which is not translated into a functional peptide or protein when it forms part of a non-coding RNA. Non-coding sequences include but are not limited to introns or parts thereof, promoter regions or parts thereof, 3′ untranslated regions (3′ UTR) or parts thereof, 5′ untranslated regions (5′ UTR) or parts thereof, as well as intergenic regions. In general, a 3′ or 5′ untranslated region is part of or spans one or more exons.

The term “coding region” or “protein-coding region” as used herein, refers to a portion of the nucleic acid sequence, which is transcribed and translated in a sequence-specific manner to produce a particular polypeptide or protein when placed under the control of appropriate regulatory sequences and appropriate molecular machinery. The coding region of a protein-coding gene is said to encode one, or more, such polypeptide or protein.

The term “oligonucleotide,” as used herein is defined as a nucleic acid molecule, or its sequence representation, comprised of at least two or more ribo- or deoxyribonucleotides. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and the method used. For example, for diagnostic applications, depending on the complexity of the target sequence, an oligonucleotide probe typically contains 15-25 or more nucleotides, although it may contain fewer nucleotides. The probes as disclosed herein are selected to be “substantially complementary” to different strands of a particular target nucleic acid sequence. This means that the probes must be sufficiently complementary so as to be able to “specifically hybridize” or anneal with their respective target strands. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarily with the sequence of the target nucleic acid to anneal therewith specifically.

In the context of this disclosure, the term “probe” refers to a molecule that can detectably distinguish among target molecules differing in sequence composition and also in structure (e.g., nucleic acid or protein sequence). Detection can be accomplished in a variety of different ways depending on the type of probe used and the type of target molecule. Thus, for example, detection may be based on discrimination on detection of specific binding. Examples of such specific binding include antibody binding and nucleic acid, antibody binding to protein, nucleic acid binding to nucleic acid, or aptamer binding to protein or nucleic acid. Thus, for example, probes can include enzyme substrates, antibodies and antibody fragments, and preferably nucleic acid hybridization probes.

The term “specifically hybridize” refers to the association between two single-stranded nucleic acid molecules of sufficient complementary sequence to permit such hybridization under pre-determined conditions generally used in the art (sometimes the sequences are referred to as “substantially complementary”). In particular, the term specifically hybridize also refers to hybridization of an oligonucleotide with a substantially complementary sequence as compared to non-complementary sequence.

The term “specifically” as used herein with reference to a probe which is used to specifically detect a given sequence of contiguous nucleotides, refers to a probe that identifies the particular sequence based on preferential hybridization to the sequence under consideration stringent hybridization conditions and/or on exclusive amplification or replication of molecules of interest.

The term “specifically” as used herein with reference to a probe which is used to specifically detect a sequence difference, refers to a probe that identifies a particular sequence difference based on exclusive hybridization to the sequence difference under stringent hybridization conditions and/or on exclusive amplification or replication of the sequence difference.

In its broadest sense, the term “substantially” as used herein in respect to “substantially complementary”, or when used herein with respect to a nucleotide sequence in relation to a reference or a target nucleotide sequence, means a nucleotide sequence having a percentage of identity between the substantially complementary nucleotide sequence and the exact complementary sequence of said reference or target nucleotide sequence of at least 60%, at least 70%, at least 80% or 85%, at least 90%, at least 93%, at least 95% or 96%, at least 97% or 98%, at least 99% or 100% (the latter being equivalent to the term “identical” in this context). For example, identity is assessed over a length of at least 10 nucleotides, or at least 11, 12, 13, 14, 15, 16, 17, 18, 19, 20, 21, 22 or up to 50 nucleotides of the entire length of the nucleic acid sequence to said reference sequence (if not specified otherwise below). Sequence comparisons can be carried out using default GAP analysis with the University of Wisconsin GCG, SEQWEB application of GAP, based on the algorithm of Needleman and Wunsch (1970, J Mol. Biol. 48: 443-453), or any of the tools that have been used for this purpose by the skilled artisan. A nucleotide sequence “substantially complementary” to a reference nucleotide sequence hybridizes to the reference nucleotide sequence under low stringency conditions, preferably medium stringency conditions, most preferably high stringency conditions.

In its broadest sense, the term “substantially identical,” when used herein with respect to a nucleotide sequence, means a nucleotide sequence corresponding to a reference or target nucleotide sequence, wherein the percentage of identity between the substantially identical nucleotide sequence and the reference or target nucleotide sequence is at least 60%, at least 70%, at least 80% or 85%, at least 90%, at least 93%, at least 95% or 96%, at least 97% or 98%, at least 99% or 100% (the latter being equivalent to the term “identical” in this context). For example, identity is assessed over a length of 10-40 nucleotides, such as at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or up to 50 nucleotides of a nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (1970, J Mol. Biol. 48: 443-453), or similar tools, as mentioned above. A nucleotide sequence “substantially identical” to a reference nucleotide sequence hybridizes to the exact complementary sequence of the reference nucleotide sequence (i.e., its corresponding strand in a double-stranded molecule) under low stringency conditions, preferably medium stringency conditions, most preferably high stringency conditions (as defined above). Homologues of a specific nucleotide sequence include nucleotide sequences that is at least 24% identical, at least 35% identical, at least 50% identical, at least 65%> identical to the reference sequence, as measured using the parameters described above, wherein the molecule represented by the homologous sequence is considered to have the same biological activity as the molecule encoded by the specific nucleotide sequence. The term “substantially non-identical” refers to a nucleotide sequence that does not hybridize to the nucleic acid sequence under stringent conditions.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length, but can be longer as needed. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

In some embodiments, the term “complementary” as used herein refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine (for DNA) or uracil (for RNA). Similarly, it is known that a cytosine residue of a first nucleic acid strand is capable of base pairing with a residue of a second nucleic acid strand which is anti-parallel to the first strand if the residue is guanine. A cytosine residue of a first nucleic acid strand is also capable of base pairing with a residue of a second nucleic acid strand which is anti-parallel to the first strand if the residue is uracil—such interactions are referred to as “non-Watson-Crick” or “G:U wobbles.” A first region of a nucleic acid is complementary to a second region of the same or a different nucleic acid if at least one nucleotide residue of the first region is capable of base pairing with a residue of the second region, when the two regions are arranged in an anti-parallel fashion.

In particular, the first region comprises a first portion and the second region comprises a second portion, whereby, when the first and second portions are arranged in an anti-parallel fashion, such that at least about 50%, and preferably at least about 75%, at least about 90%, or at least about 95% or at least 100% of the nucleotide residues of the first portion are capable of base pairing with nucleotide residues in the second portion. More preferably, all nucleotide residues of the first portion are capable of base pairing with nucleotide residues in the second portion. A first region of a nucleic acid is “near-complementary” to a second region of the same or a different nucleic acid if, at least one nucleotide residue of the first region is capable of base pairing with a residue of the second region, when the two regions are arranged in an anti-parallel fashion, and not all of the nucleotides of the two regions are base-paired. Such interactions are exemplified by heteroduplexes of miRNAs with mRNAs where the typical interaction between the two molecules is effected by only a subset of the residues spanning each region. Additionally, the two interacting regions need not have the same length.

The term “antisense oligonucleotide” refers to a nucleotide sequence including a “region of complementarity” that is substantially complementary to a sequence, for example a target sequence. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus.

As used herein, the term “antisense oligonucleotides comprising a nucleotide sequence” refers to an antisense oligonucleotide comprising a chain of nucleotides that is described by the sequence referred to using the standard nucleotide nomenclature.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured herein by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods described herein.

### X. Examples

The following examples are included to demonstrate particular embodiments. It should be appreciated by those of skill in the art that the techniques disclosed in the examples that follow represent techniques discovered by the inventors to function well in the practice of embodiments, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Cell Culure.

COX cells were obtained from the International Histocompatibility Working Group, Seattle, Wash. [(IHW09022) wordl-wide-web at ihwg.org/hla/index.html]. PGF cells were obtained from the Coriell Biorepository (Cat # GM03107). Cells were cultured in RPMI-1640 medium with 15% FBS (Sigma Cat # F2442-500 ML).

Identifying Novel miRNA Transcripts of the MHC.

Total RNA was extracted from two biological replicates (separate cell cultures collected at two individual time points) of PGF (n=2) and COX (n=2) cells using the Qiagen miRNeasy kit (Cat #217084) per manufacturer's protocol. RNA was quantified on a Nanodrop ND-100 spectrophotometer, followed by RNA quality assessment on an Agilent 2200 TapeStation (Agilent Technologies, Palo Alto, Calif.). Library construction, workflow analysis and sequencing runs were performed following standard Illumina TruSeq Small RNA protocol (15004197 Revision G). 50-base-pair single-end reads were generated on the Illumina NextSeq 500 sequencing platform and stored in FASTQ format.

Raw sequencing reads from each cell line were mapped to a haplotype specific version of the reference genome (HG38) using Novoalign, allowing up to one mismatch per read as compared to the reference genome. Reads generated from PGF were mapped to the reference genome (GRCh38) excluding all other MHC haplotype assemblies. Reads generated from COX were mapped to the reference genome (GRCh38) excluding all other MHC haplotypes, a masked MHC (chr6:28,510,019-33,383,765) and the COX MHC haplotype so as to force reads all relevant reads onto the COX MHC haplotype sequence.

Novel miRNA were identified from mapped reads using miRDeep* (version 36) (An et al., 2013) generated from each biological replicate of PGF (n=2) and COX (n=2) cell lines independently. The following miRDeep* parameters were used: minimum phred score of 15, miRNA length was set to 16-28 (length range of miRNA within miRBase release 21 (Kozomara and Griffiths-Jones, 2014), max multi-map of 500, minimum score of −15 and minimum read depth of 1. Novel miRNA were then further filtered, to include only those that are significantly expressed within each sequencing run, utilizing a previously implemented method (Londin et al., 2015).

Supporting Functional Evidence for Identified Novel mRNAs.

41 Argonaut (Ago) CLIP-seq datasets from 4 independent studies (Boudreau et al., 2014; Erhard et al., 2014; Pillai et al., 2014; Gillen et al., 2016) were interrogated for the presence of the 89 identified novel mature miRNA sequences. The raw data (fastq) files from each dataset were interrogated in order to find the number of Ago supporting reads for each of the 89 novel miRNA identified by our analysis. Only reads containing the exact, ungapped sequence of each mature miRNA were considered as supporting reads. The number of Ago CLIP-seq reads supporting each novel miRNA was tabulated. Only those miRNAs that were supported by 500 reads or more reads within all datasets were considered to be Ago Supported.

Dicer silencing was performed by designing a small hairpin RNA (shRNA) vector, which was subsequently transfected into a lentiviral plasmid for transduction into COX cells, effectively silencing Dicer expression by RNA interference (RNAi) within COX cells. The lentiviral plasmid containing the Dicer shRNA insert (GeneCopoeia catalog # HSH066175) was generated in cultured HEK293T cells by transfecting with a psi-LVRH1GP vector (GeneCopoeia). A lentiviral plasmid containing a “scrambled” sequence insert (i.e., random sequence changes all the bases) was similarly generated in HEK293T cells. Media was discarded after 24 hours post-transfection and packaging media was added to the plate. Scrambled and shRNA Dicer viruses were collected every 24 hours for 2 days. For transduction, 1.5×10⁵ COX cells were plated in 6 well plates, and 2 ml of fresh scrambled or Dicer silencing lentivirus was added along with 4 mg/ml polybrene. The plate was centrifuged at 2500 rpm for 90 minutes. After 10 hours, 2 ml of additional virus with polybrene was added and the plate was centrifuged at 2500 rpm for 90 minutes. After 16 hours, 2 ml of media was discarded and 2 ml of fresh virus and polybrene were added, and the plate was centrifuged at 2500 rpm for 90 minutes. Transduction was allowed to continue for an additional 24 hours before cells were collected for RNA extraction. RNA was extracted using the miRNeasy kit (Qiagen). Total RNA was reverse transcribed using the Qiagen miScript II RT kit (Cat #218160), with either 1) the miScript HiFlex Buffer (for quantification of Dicer mRNA) or 2) MiScript HiSpec Buffer (for specific quantification of mature miRNA only). Q-PCR was performed on cDNA generated by reverse transcription using a miSCRIPT SYBR Green PCR kit (Cat #21803). For the purposes of validating Dicer silencing, cDNA generated using the MiScript HiFlex buffer was quantified with Dicer specific qPCR primers (forward primer sequence: 5′-TAACCTTTTGGTGTTTGATGAGTGT-3′ (SEQ ID NO: 90), reverse primer sequence: 5′-GGACATGATGGACAATTTCACA-3′(SEQ ID NO: 91)). For the purpose of assessing the quantities of mature miRNA in both Dicer silencing and scrambled control conditions, cDNA generated using the MiScript HiSpec Buffer was quantified using miRNA specific primers (mature miRNA DNA sequences). Primers were obtained from IDT. Two biological replicates for each condition were performed (silencing and control) and p-values were calculated using a t-test on normalized (beta-actin) ΔCt values (Yuan et al., 2006). A p-value less than 0.05 was deemed significant.

Haplotype Conservation of Novel miRNAs.

Novel miRNA sequence conservation across annotated MHC haplotype sequences was interrogated using BLAST. All eight available MHC haplotype sequences (PGF, COX, APD, SSTO, QBL, DBB, MANN and MCF) were scanned for the existence of every identified mature and pre-miRNA sequence (Supplemental Table 1) using BLAST (version 2.4.0+; parameters:—task megablast—word_size 7-evalue 1000) (Camacho et al., 2009). Only perfectly matched sequences (100% sequence identity between the query sequence and the reference MHC haplotype) were considered to be conserved.

Sequence Homology of Novel miRNAs to Known miRNAs.

The target specificity of a miRNA transcript is primarily determined by its sequence, which directs the formation of an energetically favorable double stranded RNA heteroduplex between the miRNA and a complementary RNA target sequence (Xia et al., 2012; Helwak et al., 2013). Consequently, sequence homology amongst miRNA transcripts may be indicative of a shared target repertoire and redundant physiological function. In order to evaluate the sequence homology between each of the identified novel miRNA transcripts (n=89) and all annotated miRNA within miRBase (release 21), each novel miRNA sequence was aligned pairwise with every annotated miRNA sequence using the semi-global Needleman-Wunsch algorithm implemented within MATLAB (2014a). For each identified novel miRNA, the closest matched annotated miRNA (highest alignment score) was reported. In the case in which a novel miRNA aligned to multiple annotated miRNA with the same maximal alignment score, every match was reported (Supplemental Table 2).

In Silico Discovery of Putative Pre-mIRNA Encoding Loci.

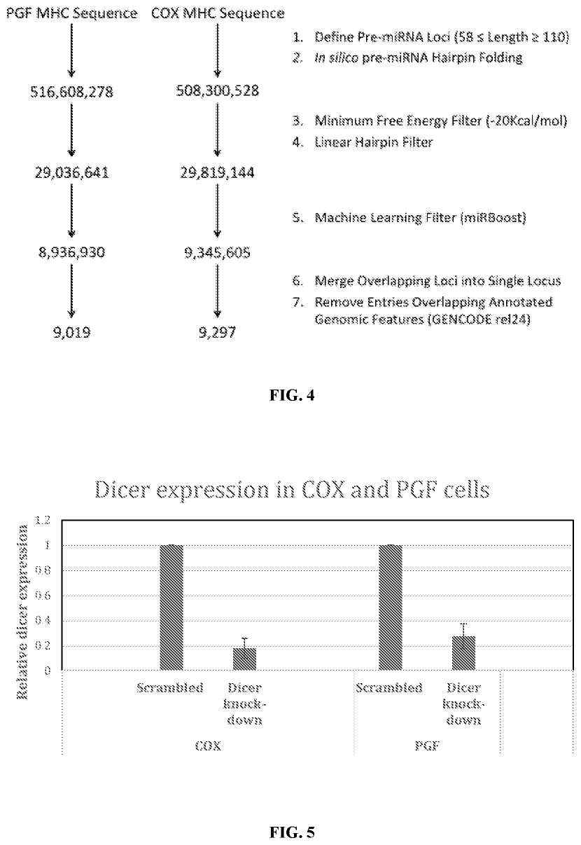

A computational pipeline was developed (FIG. 4) in order to identify every putative pre-miRNA encoding locus present within the reference MHC haplotype sequences of both PGF and COX (Stewart et al., 2004). The developed pipeline takes a FASTA file as input, which was generated from the reference genome (HG19) using BEDtools (Quinlan and Hall, 2010; Quinlan 2014) and ranged from HG38 coordinates chr6:28510019-33383765 and chr6_cox_hap2:1-4795371 for PGF and COX haplotypes respectively. The pipeline begins by first partitioning the FASTA file provided as input into overlapping segments of variable length using a 1 bp sliding window (58≤Length≥110), generating sequences from both the forward and reverse strands (output in the 5′->3′ direction) for each iteration so as to enumerate every possible pre-miRNA encoding locus throughout the MHC. In silico RNA folding was subsequently performed for each putative pre-miRNA transcript to determine the minimum free energy (MFE) and secondary structure of each theoretical transcript using RNAfold (Lorenz et al., 2011). The acceptable parameter range for both window length (pre-miRNA transcript length) and MFE were selected as the 95^{th} and 5^{th} percentile value for both distributions of annotated human pre-miRNA within miRbase release 21 (n=1,881) (Kozomara and Griffiths-Jones, 2014), corresponding to a length of 58-110 bp and a maximum MFE of −20 Kcal/mol respectively. In order to further reduce the search space, pre-miRNA hairpin structures containing bulge loops or multi-stem structures and those with a MFE≥−20 Kcal/mol were removed, leaving only characteristic linear pre-miRNA hairpin with a permissible MFE. The machine-learning algorithm, miRBoost (Tran Vdu et al., 2015) was then utilized to identify high confidence pre-miRNA hairpin structures. The miRBoost training set was first generated by running gFold (Reidys et al., 2011) on positive and negative control datasets, which comprised of the human pre-miRNA present within miRBase (release 21) and the provided internal miRBoost negative Human control dataset respectively. Lastly, the BED formatted output file was subsequently filtered to remove any entries that overlap an annotated exon (GENCODE v24) (Harrow et al., 2012) and were then merged so as to remove overlapping entries and create an atlas of loci throughout the MHC that contain at least one putative pre-miRNA hairpin locus.

BLAST was used to determine sequence homology amongst computationally predicted pre-miRNA encoding loci identified from the in silico analysis of both the PGF (n=9019) and COX (n=9207) MHC haplotypes. The sequences of each putative pre-miRNA encoding loci identified from both haplotypes were aligned pairwise using BLAST and filtered to include only those loci whose sequences matched 100% (minimum overlap defined by the shortest of the two compared sequences).

Novel miRNA Loci within LD of Disease Associated SNPs.

Annotated disease associated SNPs within the MHC were collected from the GWAS catalog (world-wide-web at ebi.ac.uk/gwas, accessed on Mar. 1, 2017) (Welter et al., 2014; MacArthur et al., 2017). The linkage disequilibrium (LD) block of each disease associated SNP was calculated using SNAP (HapMap release 22) with a minimum r² of 0.9 (Johnson et al., 2008). The LD blocks defined by each disease associated SNP were then intersected with the set of empirically derived novel miRNA as well as the set of identified computationally predicted pre-miRNA encoding loci using BEDtools (Quinlan and Hall, 2010; Quinlan, 2014) in order to determine which novel miRNA encoding loci lie within the LD block of each disease associated SNP.

### Example 2—Results

Identifying Novel mIRNA Transcripts of the MHC.

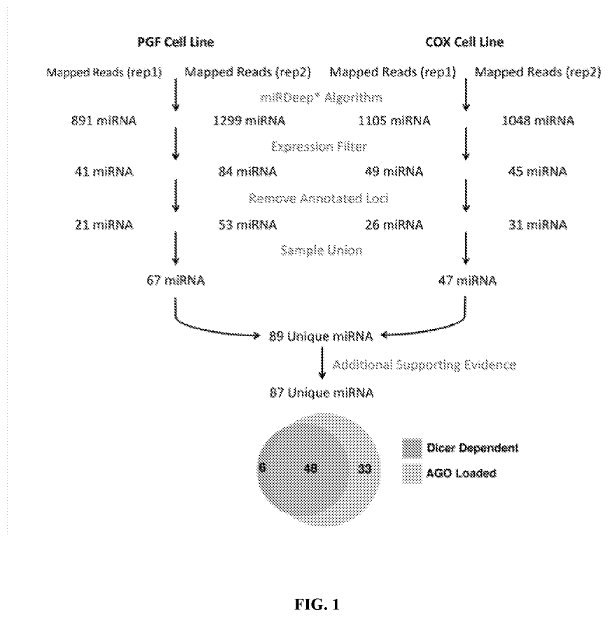

Deep sequencing of the miRNA transcriptome was performed on two BLCLs, PGF and COX. These two cell lines were chosen because they are homozygous for the MHC region and both have distinct and completely sequenced MHC haplotypes, facilitating unambiguous mapping of short RNA-seq reads to polymorphic regions throughout the MHC. Although the genome-wide transcriptional profile of BLCLs has been previously reported (Jima et al., 2010; Londin et al., 2015), these efforts do not adequately describe the profile of haplotype and allele specific miRNA transcripts originating from within the polymorphic MHC. For this reason, our experimental design and analysis pipeline were developed, facilitating the identification and characterization of novel miRNA transcripts within the MHC. A modified version of a previously established analytical pipeline (Londin et al., 2015) was utilized for the discovery of novel miRNA encoded within the MHC using mapped RNA-seq reads from the two biological replicates of each cell line (FIG. 1A).

Two biological replicates of each cell line were sequenced, generating a total of four RNA-seq datasets. The reads from each sample were mapped to the reference genome (HG38) that had modified to include either the PGF or COX MHC haplotype reference sequence, depending on the cell line of origin. Approximately 90% of the raw reads generated by each sequencing run were aligned to their respective haplotype specific reference genome. The set of aligned reads was subsequently used for the identification of novel miRNA transcripts using the miRDeep* algorithm (An et al., 2013). Only the identified, novel miRNA that were significantly expressed within each individual sequencing run and did not overlap with an annotated miRNA (mirBase release 21) or exonic, protein coding sequence were retained for further analysis. In total, 89 novel mature miRNA were identified from the analysis of RNA-Seq data obtained from the two cell lines. The majority (82%) of identified novel miRNA lie within intergenic regions of the MHC, with 16 identified novel mature miRNA residing within the introns of 14 unique genes; ATF6B, C2, CSNK2B, DDX39B, GABBR1, HGC20, HLA-DRB5, LY6G6C, MSH5, NFKBIL1, NOTCH4, SLC39A7, TNXB, and TRIM31.

Supporting Functional Evidence for Identified Novel miRNAs.

Since the majority of mature miRNA transcripts are formed through the canonical Dicer-dependent biogenesis pathway (Chakravarthy et al., 2010; Feng et al., 2012; Taylor et al., 2013), Dicer knockdown or silencing experiments have been used to identify mature miRNA that are formed in a Dicer-dependent manner (Ambros et al., 2003). Our Dicer silencing experiment demonstrates that the expression of 54/89 (60%) of the identified novel miRNAs are significantly attenuated following Dicer silencing (pval≤0.05) as evaluated by qPCR, suggesting that the biogenesis of these mature miRNAs is Dicer dependent.

Mature miRNAs facilitate the suppression of targeted mRNA transcripts following miRNA loading onto the Ago RISC (Kobayashi and Tomari, 2016). For this reason, Ago CLIP-seq datasets, in which the Ago protein is immunoprecipitated and the bound RNA fraction sequenced, have been previously used to identify functional miRNA targets and validate functional novel miRNAs (Friedlander et al., 2014; Londin et al., 2015). The inventors performed a meta-analysis on 41 Ago CLIP-seq datasets from 4 independent studies (Boudreau et al., 2014; Erhard et al., 2014; Pillai et al., 2014; Gillen et al., 2016) in order to provide evidence that our 89 novel miRNA transcripts are functional miRNA that are loaded onto the Ago silencing complex. Our results indicate that 81 of the 89 (91%) identified novel miRNA described by this study are found to be loaded onto the Ago silencing complex. These results along with the Dicer silencing results demonstrate that 48/89 of the novel miRNA were found to be formed in a Dicer dependent manner and also loaded onto the Ago silencing complex. The genomic locus they originate from and supporting evidence (Ago support and Dicer dependency) for each identified novel mature miRNA is provided in Supplemental Table 1.

Haplotype Conservation of Novel miRNAs.

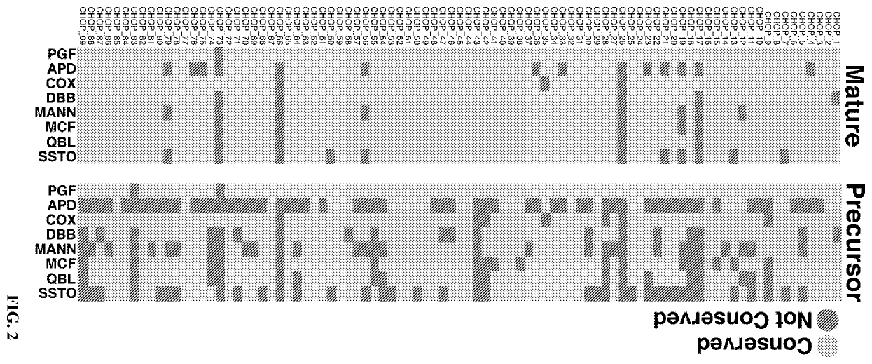

In order to determine the presence of each identified novel mature and pre-miRNA encoding sequence within the set of annotated complete (PGF and COX) as well as partially complete MHC haplotypes (MCF, DBB, MANN, APD, SSTO and QBL), each miRNA sequence was compared with each annotated MHC haplotype sequence using BLAST. Only perfectly matched sequences (100% sequence identity between the query sequence and the reference MHC haplotype) were considered to be conserved within a particular MHC haplotype (FIG. 2). Our results indicate that while the majority of mature miRNA are conserved amongst the set of eight annotated MHC haplotypes analyzed, a subset of mature miRNAs are found to exist within specific MHC haplotypes (FIG. 2). Of note CHOP_66 lies within intron 5 of HLA-DRB5 and is composed of reads that map uniquely to this locus. It was also observed that the identified pre-miRNA sequences are less conserved across the analyzed haplotypes as compared to the mature miRNA sequences (FIG. 2).

Sequence Homology of Novel miRNAs to Known mRNAs.

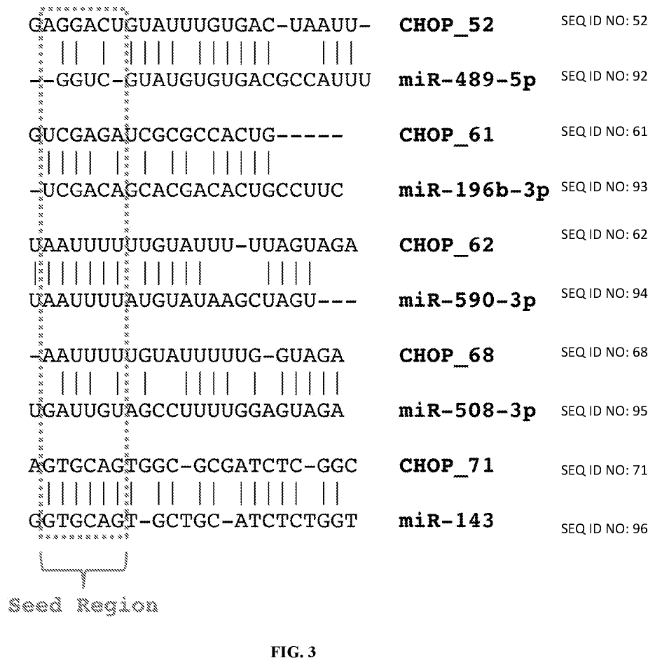

The set of identified 89 novel mature miRNA were compared against all known, previously annotated miRNA (miRBase release 21) in order to identify closely related miRNAs that share partial sequence homology, which may be indicative of a shared target repertoire and physiological function. For this purpose, each identified novel miRNA was aligned pairwise with every annotated mature miRNA sequences (miRBase release 21). Several identified novel miRNAs closely matched the sequences of annotated miRNAs of known physiological function that have been demonstrated to play a role in oncogenesis (FIG. 3), including miR-489 (Chai et al., 2016), miR-196 (Popovic et al., 2009; Lu et al., 2014), miR-590 (Chu et al., 2014), miR-508 (Shang et al., 2014) and miR-143 (Ng et al., 2014).

In Silico Discovery of Putative Pre-miRNA Encoding Loci.

Many recently discovered miRNA, including those described within our current work, rely on the identification of novel miRNA from RNA-seq data derived from a variety of tissue types. However, such an approach is inherently limited to discovering only the set of expressed miRNA transcribed within the interrogated tissue types. Given the demonstrated tissue specific RNA expression patterns and limited number of cell lines with completely characterized MHC haplotypes, the inventors have developed a computational pipeline to identify all putative pre-miRNA encoding loci within the reference MHC haplotype sequences of both PGF and COX that may be expressed by various cell types and developmental stages. The developed pipeline is a multi-step process designed to exhaustively interrogate the propensity of genomic loci to form stable, pre-miRNA hairpin structures (FIG. 4). Our analysis identified 9,019 and 9,297 loci containing at least one pre-miRNA hairpin structure for PGF and COX MHC haplotypes respectively. The sequences of 4,487 of the putative pre-miRNA encoding loci identified within the PGF MHC haplotype are 100% conserved within the COX MHC haplotype (4,487/9,019, ˜50%). Overall, 11 of the 12 annotated pre-miRNAs (miRbase release 21) located within the MHC were also identified by our computational analysis pipeline. One miRNA, hsa-miR-6833 was filtered out because the pre-miRNA hairpin was found to have a minimum free energy (MFE) of −19.1 Kcal/mol, which is greater (less energetically favorable) than the cutoff threshold of −20 Kcal/mol. Furthermore 80 of the 89 (90%) pre-miRNA identified through the analysis of RNA-Seq datasets, were also identified through our ab initio computational pre-miRNA annotation pipeline.

Novel miRNA Loci within LD of Disease Associated SNPs.

Although elucidating the physiological function of each identified novel miRNA is beyond the scope of this work, the inventors seek to provide some insights into the potential role of the identified novel miRNA transcripts in the context of the many diseases reported to be associated with the MHC. Utilizing the wealth of annotated disease associated variants from GWAS studies (Welter et al., 2014; MacArthur et al., 2017), the inventors identified the subset of the miRNAs that lie within LD blocks of annotated disease associated variants within the MHC. Our results indicate that 43 of the 89 (˜48%) identified novel miRNA transcripts from the analysis of RNA-seq datasets are within LD blocks containing 87 unique disease associated SNPs. These 87 SNPs have been associated with 65 unique phenotypes (Supplemental Table 3). In addition, 6,690 computationally derived putative pre-miRNA encoding loci identified from the analysis of the PGF MHC haplotype sequence were found to be in LD with 325 unique associated SNPs (data not shown).

### Example 3—Discussion

Deep RNA sequencing of the miRNA transcriptome from two BLCLs with completely characterized MHC haplotypes (PGF and COX) has enabled the accurate alignment of short RNA-seq reads to each respective MHC haplotype sequence, facilitating the identification of 89 novel miRNA transcripts originating from within the polymorphic MHC region (Supplemental Table 1). Additional experimental validation of the set of identified novel miRNA transcripts demonstrates that 54/89 (˜60%) of the identified novel miRNA transcripts are significantly attenuated (p≤0.05) following Dicer silencing and that 81/89 (˜91%) are loaded onto the Ago silencing complex. Together these data demonstrate that 54% (48/89) of the identified novel miRNA are functional miRNA that are formed through the canonical, Dicer-dependent biogenesis pathway (Ambros et al., 2003) and are loaded onto the Ago silencing complex. The number of identified novel miRNA that undergo Dicer dependent biogenesis may however be an underestimate, since previous research demonstrates that functional mature miRNA transcripts can be formed independently of the Dicer enzyme (Cheloufi et al., 2010; Cifuentes et al., 2010; Yang and Lai, 2010). Alternatively, the lower than expected number of identified miRNAs that undergo Dicer dependent biogenesis may be attributed to 1) accumulation of mature miRNA transcripts prior to Dicer silencing, 2) differential transcription of pre-miRNA hairpin transcripts amongst the two biological replicates or 3) differential Dicer processing of pre-miRNA hairpins (Feng et al., 2012; Ple et al., 2012), resulting in the expression of miRNA isoforms (isomiRs) that may not be an optimal qPCR substrate for amplification using our designed set of primers.

The majority of identified miRNA (73/89) originate from within the intergenic regions of the MHC, while 16/89 are located within an intron of an annotated host gene. The inventors also find that 12/73 identified intergenic miRNAs are antisense to an annotated protein-coding gene. The mature miRNAs that lie within an intron, “mirtrons”, are formed following splicing of the mRNA transcript and have been shown to exist throughout the human genome (Berezikov et al., 2007; Ruby et al., 2007; Ladewig et al., 2012). Our previous research demonstrates that one such mirtron, miR-6891-5p, which is encoded within intron 4 of the HLA-B gene, plays an important physiological role by regulating the post transcriptional expression of nearly 200 mRNA transcripts that are involved in a variety of metabolic and immunological processes (Chitnis et al., 2016). Interestingly, our current work suggests that HLA-DRB5 also harbors an observed mirtron transcript, located within intron 5 of the HLA-DRB5 gene. In addition, the inventors find numerous pre-miRNA hairpins located within other HLA genes as predicted by our in silico analysis. Together these data suggest a novel, secondary function for select HLA transcripts, mediated by their encoded miRNAs.

The existence of MHC encoded miRNA raises many questions related to the existence and prevalence of haplotype specific miRNAs. Although the majority of identified mature miRNA sequences are conserved across all eight known MHC haplotypes, several mature miRNA and pre-miRNA hairpin sequences are found to be unique to specific MHC haplotypes (FIG. 2). It is however important to note that only the MHC haplotypes of PGF and COX have been fully characterized, potentially resulting in a miRNA sequence being absent within a particular MHC haplotype simply because it lies within an uncharacterized region of that particular haplotype. For this reason, mature mIRNA were also intersected with common SNPs (dbSNP build 149), revealing that 27/89 (minor allele frequency—MAF≥0.01) and 16/89 (MAF≥0.05) of the identified novel mature miRNA transcripts contain at least one common SNP and are thus likely to be polymorphic across the population. Although the majority of identified novel mature miRNA sequences are conserved across most MHC haplotypes, the pre-miRNA hairpin sequences are far less conserved across MHC haplotypes. Polymorphisms within the pre-miRNA transcripts can influence the free energy and conformation of the secondary structure of the pre-miRNA hairpin structure, thereby influencing pre-miRNA processing by Dicer, which may result in a variety of expressed isomiRs (Ple et al., 2012; Ma et al., 2016; Liang et al., 2017). Furthermore, an assessment of the haplotype conservation amongst the set of identified computationally predicted putative pre-miRNA encoding loci, reveals that ˜50% (4,487/9,019) of these sequences are 100% conserved between the PGF and COX MHC haplotypes. Together these data suggest a diverse miRNA transcriptome, with the potential for a variety of isomiR transcripts that are defined by inherent differences amongst MHC haplotypes. It is anticipated that each of these isomiRs has a distinct target repertoire governed by the sequence specific interaction of the mature miRNA with targeted mRNA transcripts (Ple et al., 2012; Loher et al., 2014).

The availability of an accurate, reference genome sequence is critical to ensure accurate mapping of short RNA-seq reads for the identification and quantification of novel miRNAs and isomiRs that are transcribed from within highly polymorphic loci such as the MHC. Because there are only two complete MHC haplotype sequences currently available (PGF and COX), our study is limited to the identification and quantification of novel miRNAs expressed by these two cell lines. However, because miRNA and isomiRs are expressed in a tissue and phenotype specific manner (Loher et al., 2014; Fehlmann et al., 2016; Ludwig et al., 2016), the inventors sought to identify every potential miRNA encoding locus throughout the MHC using only the DNA reference haplotype sequences of PGF and COX as a guide. Our computational pipeline has identified thousands of potential pre-miRNA encoding loci throughout the MHC and serves as an atlas for the future identification and quantification of MHC encoded miRNA transcripts, which may be expressed by various tissue types, cellular phenotypes and developmental stages. Previous research suggests that there may be as many as 55,000 pre-miRNA encoding loci throughout the human genome (Miranda et al., 2006), which would mean, if equally distributed, that the MHC would be expected to harbor ˜74 pre-miRNA hairpins. However, our computational estimates suggests the existence of many more pre-miRNA hairpins than anticipated within the MHC, suggesting that the gene dense MHC may harbor more miRNA than previously calculated. It is only after the inventors are able to characterize the totality of miRNA transcripts across a variety of tissue types, diseases and developmental states from diverse populations that they can begin to understand the full spectrum of miRNA diversity within the MHC. In order to properly study allele and haplotype specific miRNA expression patterns of MHC encoded miRNAs, it is first necessary to resolve the sequence of an individual's MHC haplotype so that expressed transcripts can be accurately mapped to an individual's MHC haplotype sequence. Working toward this goal, the inventors have developed a long fragment DNA enrichment method (Dapprich et al., 2016), capable of generating long DNA fragments that may be utilized by single molecule sequencing platforms to generate long reads, which has been previously utilized by our group for de novo assembly of the MHC (Clark et al., 2015a). These efforts lay the foundation for studying allele and haplotype specific transcript expression patterns across diverse sample populations and may be extended to any portion of the genome of interest.

Although determining the functional significance of each identified novel miRNA is beyond the scope of our current work, our data suggest that a subset of identified novel miRNA lie within LD of a disease associated SNP. It should also be noted that the vast majority of disease-associated variants found to be in LD with novel miRNA are located within non-coding regions of the MHC. Considering that 90% of causal autoimmune disease variants reside within non-coding regions of the genome (Farh et al., 2015), it is possible that these miRNA may play a role in the pathophysiology of the associated disease and warrants further experimental investigation. Furthermore, five of the identified novel miRNAs share considerable sequence homology with oncogenic miRNAs (oncomiRs) including miR-590 (Chu et al., 2014), miR-196 (Popovic et al., 2009; Lu et al., 2014), miR-489 (Chai et al., 2016), miR-508 (Shang et al., 2014) and miR-143 (Ng et al., 2014). These novel miRNA are shown to have a high degree of sequence homology with already known and previously described annotated miRNAs (FIG. 3) and may be indicative of a shared target repertoire and redundant physiological function. Together, these data suggest that a subset of the identified novel miRNA may contribute to the pathophysiology of the numerous diseases, laying the groundwork for future studies to elucidate the functional connections of miRNAs to the numerous diseases associated with sequence variants within non-coding regions of the MHC.

SUPPLEMENTAL TABLE 1

| Novel | miRDee | | | | | | SEQ ID | Dicer | AGO |
|---|---|---|---|---|---|---|---|---|---|
| miRNA ID | p* Score | chr | strand | hairPin_loci | mature_loci | Mature miRNA Sequence | NO: | Dependent | Supported |
| CHOP_1 | −1.64 | chr6 | − | 28869349-28869431 | 28869398-28869421 | aacccaggaggcggaacuugcagu | 1 | Yes | Yes |
| CHOP_2 | 0.26 | chr6 | − | 31533661-31533735 | 31533671-31533690 | aauagccacugcacuccagc | 2 | Yes | Yes |
| CHOP_3 | −3.39 | chr6 | − | 30593611-30593683 | 30593621-30593639 | acccaggaggcagagguug | 3 | | Yes |
| CHOP_4 | −1.2 | chr6 | + | 31686084-31686168 | 31686134-31686158 | acugcacuccagccugggcaacaua | 4 | Yes | Yes |
| CHOP_5 | −3.49 | chr6 | − | 33373716-33373786 | 33373759-33373776 | aggcuggagugcaauggc | 5 | Yes | Yes |
| CHOP_6 | 0.08 | chr6 | + | 31922603-31922681 | 31922613-31922634 | agguggaucaccugaggucagg | 6 | | Yes |
| CHOP_7 | −9.36 | chr6 | − | 32078526-32078594 | 32078536-32078552 | aguacuuggaugggaga | 7 | | Yes |
| CHOP_8 | −7.61 | chr6 | + | 30122198-30122276 | 30122245-30122266 | aguucgagaccagccugggcaa | 8 | | Yes |
| CHOP_9 | 109.99 | chr6 | + | 30773502-30773578 | 30773548-30773568 | cacugcacuccagccugggca | 9 | | Yes |
| CHOP_10 | −1.4 | chr6 | + | 33226125-33226203 | 33226172-33226193 | cacugcacucuagccugggcga | 10 | | Yes |
| CHOP_11 | −10.07 | chr6 | − | 32118342-32118412 | 32118385-32118402 | caggcuggucucgaacuc | 11 | | Yes |
| CHOP_12 | −2.83 | chr6 | + | 31787739-31787813 | 31787784-31787803 | cugggcaacauagcgagacc | 12 | | Yes |
| CHOP_13 | 1.34 | chr6 | − | 30111222-30111296 | 30111232-30111251 | cugggcaacauagcgagacu | 13 | Yes | Yes |
| CHOP_14 | 103.45 | chr6 | + | 31757155-31757225 | 31757198-31757215 | gcaguggcgcgaucucgg | 14 | | Yes |
| CHOP_15 | 349.19 | chr6 | + | 32981887-32981957 | 32981930-32981947 | gccgagaucgcgccacug | 15 | Yes | Yes |
| CHOP_16 | −13.02 | chr6 | − | 30530520-30530600 | 30530568-30530590 | ggaggaucgcuugaguccaggag | 16 | | Yes |
| CHOP_17 | −1.37 | chr6 | − | 28673839-28673917 | 28673886-28673907 | ggggauguagcucagugguaga | 17 | | Yes |
| CHOP_18 | −2.12 | chr6 | + | 28719694-28719776 | 28719704-28719727 | ggggguguagcucagugguagagc | 18 | Yes | Yes |
| CHOP_19 | 0.76 | chr6 | + | 30955647-30955713 | 30955688-30955703 | gucccggcggagucgc | 19 | Yes | Yes |
| CHOP_20 | −4.19 | chr6 | − | 29597099-29597169 | 29597142-29597159 | guugcccaggcuggagug | 20 | Yes | Yes |
| CHOP_21 | 229.13 | chr6 | + | 33201670-33201750 | 33201718-33201740 | uacuugaccuugacucucccuca | 21 | Yes | |
| CHOP_22 | −2.86 | chr6 | + | 33368155-33368233 | 33368202-33368223 | ugaccucgugaucugcccgccu | 22 | Yes | Yes |
| CHOP_23 | −7.23 | chr6 | + | 31666746-31666830 | 31666796-31666820 | uuuacugacacuguucuuuuucuag | 23 | Yes | |
| CHOP_24 | −11.91 | chr6 | − | 28822284-28822364 | 28822332-28822354 | uuuaguagagacgggguuucacc | 24 | | Yes |
| CHOP_25 | −6.68 | chr6 | − | 30984488-30984566 | 30984498-30984519 | uuuuguauuuuuaguagagaca | 25 | Yes | Yes |
| CHOP_26 | 1.88 | chr6 | − | 32621704-32621782 | 32621714-32621735 | aauuucugcauaguccaccucu | 26 | Yes | |
| CHOP_27 | −2.15 | chr6 | + | 32060665-32060745 | 32060713-32060735 | acugcaaccucugccucccgggu | 27 | | Yes |
| CHOP_28 | −4.58 | chr6 | + | 31783638-31783708 | 31783681-31783698 | agacggagucucgcucug | 28 | | Yes |
| CHOP_29 | −4.79 | chr6 | − | 29616609-29616689 | 29616657-29616679 | agagucuugcucuguugccuagg | 29 | | Yes |
| CHOP_30 | −4.19 | chr6 | − | 33381575-33381661 | 33381585-33381610 | aggcaggagaaucacuugaacccggg | 30 | Yes | Yes |
| CHOP_31 | −2.83 | chr6 | − | 31705212-31705290 | 31705259-31705280 | aggcggaucaccugaggucagg | 31 | | Yes |
| CHOP_32 | −2.2 | chr6 | + | 31595059-31595149 | 31595112-31595139 | aggcugaggcaggagaaucacuugaacc | 32 | Yes | Yes |
| CHOP_33 | −5.82 | chr6 | + | 31555271-31555341 | 31555281-31555298 | aggcuggagugcacuggc | 33 | | Yes |
| CHOP_34 | −1.61 | chr6 | + | 29983639-29983717 | 29983686-29983707 | aguucgagaccagccugaccaa | 34 | Yes | Yes |
| CHOP_35 | −2.86 | chr6 | − | 30726532-30726608 | 30726578-30726598 | caacauagcaagacuccaucu | 35 | Yes | Yes |
| CHOP_36 | −2.2 | chr6 | + | 33219323-33219397 | 33219333-33219352 | cacccaggcuggagagcagu | 36 | | Yes |
| CHOP_37 | 0.42 | chr6 | + | 33258608-33258692 | 33258658-33258682 | cacugcaaccuccgccucccagguu | 37 | | Yes |
| CHOP_38 | 1.02 | chr6 | + | 30243415-30243491 | 30243461-30243481 | cacugcacuccagccugggcg | 38 | Yes | Yes |
| CHOP_39 | 0.62 | chr6 | + | 31527950-31528030 | 31527998-31528020 | cagccugggcaacagagcgagac | 39 | Yes | Yes |
| CHOP_40 | −1.68 | chr6 | − | 32893643-32893717 | 32893688-32893707 | cagggucucgcucugucgcc | 40 | | Yes |
| CHOP_41 | 0.72 | chr6 | + | 29188931-29189015 | 29188981-29189005 | caugugucuuuauaguagaaugauu | 41 | Yes | Yes |
| CHOP_42 | 12.11 | chr6 | + | 30773501-30773577 | 30773547-30773567 | ccacugcacuccagccugggc | 42 | Yes | Yes |
| CHOP_43 | 73.62 | chr6 | − | 32441274-32441344 | 32441317-32441334 | ccaggcuggagugcagug | 43 | Yes | Yes |
| CHOP_44 | −3.35 | chr6 | − | 32885544-32885618 | 32885554-32885573 | cccagcuacucgggaggcug | 44 | Yes | Yes |
| CHOP_45 | −1.65 | chr6 | − | 32878435-32878515 | 32878483-32878505 | cccgggaggcggagcuugcagug | 45 | Yes | Yes |
| CHOP_46 | −1.27 | chr6 | − | 32203130-32203210 | 32203178-32203200 | cgggcacaguggcucacgccugu | 46 | | Yes |
| CHOP_47 | −1.63 | chr6 | − | 30902270-30902350 | 30902318-30902340 | cucacgccuguaaucccagcacc | 47 | Yes | Yes |
| CHOP_48 | −6.95 | chr6 | − | 31703910-31703982 | 31703920-31703938 | cucacugcaaccucugccu | 48 | Yes | Yes |
| CHOP_49 | −3.18 | chr6 | + | 28764408-28764488 | 28764418-28764440 | cugaagaucuaaaggucccuggu | 49 | | |
| CHOP_50 | −5.11 | chr6 | − | 28807841-28807923 | 28807851-28807874 | cugaagaucuaaaggucccugguu | 50 | Yes | |
| CHOP_51 | −3.14 | chr6 | − | 30117417-30117487 | 30117427-30117444 | cugaccucgugauccgcc | 51 | | Yes |
| CHOP_52 | 27.11 | chr6 | + | 32326294-32326374 | 32326342-32326364 | gaggacuguauuugugacuaauu | 52 | Yes | Yes |
| CHOP_53 | −13.18 | chr6 | − | 29783083-29783167 | 29783093-29783117 | gagguguuuauaguauucucugaug | 53 | | Yes |
| CHOP_54 | −4.58 | chr6 | − | 33160492-33160564 | 33160502-33160520 | gagucucgcucugucgccc | 54 | Yes | Yes |
| CHOP_55 | 56.6 | chr6 | + | 28709145-28709227 | 28709194-28709217 | gccaagaucgcgccacugcacucc | 55 | Yes | Yes |
| CHOP_56 | 0.33 | chr6 | + | 31901998-31902070 | 31902042-31902060 | gcgcgcggcggcggcggcg | 56 | | Yes |
| CHOP_57 | −1.04 | chr6 | − | 30648625-30648695 | 30648668-30648685 | gcucacgccuguaauccc | 57 | Yes | Yes |
| CHOP_58 | −7.08 | chr6 | + | 28934513-28934599 | 28934564-28934589 | gcugaggcaggagaaucgcuugaacc | 58 | Yes | Yes |
| CHOP_59 | −0.8 | chr6 | − | 28827416-28827494 | 28827463-28827484 | ggggguauagcucagugguaga | 59 | Yes | Yes |
| CHOP_60 | −8.1 | chr6 | − | 32078492-32078566 | 32078537-32078556 | gguuaguacuuggaugggag | 60 | Yes | Yes |
| CHOP_61 | −3.86 | chr6 | + | 31651475-31651545 | 31651485-31651502 | gucgagaucgcgccacug | 61 | Yes | Yes |
| CHOP_62 | −4.83 | chr6 | − | 28777850-28777930 | 28777860-28777882 | uaauuuuuuguauuuuuaguaga | 62 | | Yes |
| CHOP_63 | −2.28 | chr6 | − | 33232017-33232087 | 33232027-33232044 | ucacugcaaccuccgccu | 63 | | Yes |
| CHOP_64 | 30.55 | chr6 | + | 32124871-32124953 | 32124920-32124943 | ucacugcaagcuccgccucccggg | 64 | Yes | Yes |
| CHOP_65 | −3.8 | chr6 | − | 31719620-31719704 | 31719630-31719654 | ugaggcaggagaaucgcuugaaccu | 65 | Yes | Yes |
| CHOP_66 | −7.52 | chr6 | − | 32517790-32517868 | 32517800-32517821 | uugaaagagaggaaaagaagcu | 66 | Yes | |
| CHOP_67 | 0.26 | chr6 | − | 28752429-28752507 | 28752476-28752497 | uuggccaggcuggucucgaacu | 67 | Yes | Yes |
| CHOP_68 | −12.05 | chr6_c | + | 1203973-1204049 | 1204019-1204039 | aauuuuuguauuuuugguaga | 68 | | Yes |
| | | ox_hap | | | | | | | |
| | | 2 | | | | | | | |
| CHOP_69 | 0.33 | chr6_c | − | 4674763-4674837 | 4674808-4674827 | acccaggcuggagugcagug | 69 | | Yes |
| | | ox_hap | | | | | | | |
| | | 2 | | | | | | | |
| CHOP_70 | −1.42 | chr6_c | − | 4739873-4739953 | 4739883-4739905 | aggcaggagaauugcuugaaccc | 70 | Yes | Yes |
| | | ox_hap | | | | | | | |
| | | 2 | | | | | | | |
| CHOP_71 | −0.04 | chr6_c | − | 2173243-2173321 | 2173253-2173274 | agugcaguggcgcgaucucggc | 71 | | Yes |
| | | ox_hap | | | | | | | |
| | | 2 | | | | | | | |
| CHOP_72 | −0.9 | chr6_c | − | 3394317-3394387 | 3394360-3394377 | cccaggcuggaguacagu | 72 | Yes | Yes |
| | | ox_hap | | | | | | | |
| | | 2 | | | | | | | |
| CHOP_73 | −1.72 | chr6_c | − | 2206377-2206453 | 2206423-2206443 | cgacauagcaagacuccaucu | 73 | | |
| | | ox_hap | | | | | | | |
| | | 2 | | | | | | | |
| CHOP_74 | −2.23 | chr6_c | + | 219418-219494 | 219428-219448 | cgcccaggcuggagugcagug | 74 | | Yes |
| | | ox_hap | | | | | | | |
| | | 2 | | | | | | | |
| CHOP_75 | 1.87 | chr6_c | − | 3217879-3217955 | 3217889-3217909 | cgccccaggucucggucccug | 75 | Yes | |
| | | ox_hap | | | | | | | |
| | | 2 | | | | | | | |
| CHOP_76 | −1.48 | chr6_c | − | 2662534-2662620 | 2662544-2662569 | cugaggugggaggaucgcuugagccu | 76 | Yes | Yes |
| | | ox_hap | | | | | | | |
| | | 2 | | | | | | | |
| CHOP_77 | −7.3 | chr6_c | − | 270979-271049 | 271022-271039 | cugggacuacaggcaccc | 77 | Yes | Yes |
| | | ox_hap | | | | | | | |
| | | 2 | | | | | | | |
| CHOP_78 | −3.37 | chr6_c | − | 3229400-3229470 | 3229443-3229460 | gagucuugcucugucgcc | 78 | Yes | Yes |
| | | ox_hap | | | | | | | |
| | | 2 | | | | | | | |
| CHOP_79 | 0.33 | chr6_c | + | 3379533-3379601 | 3379575-3379591 | gcgcggcggcggcggcg | 79 | Yes | Yes |
| | | ox_hap | | | | | | | |
| | | 2 | | | | | | | |
| CHOP_80 | −3.74 | chr6_c | − | 2413252-2413318 | 2413262-2413277 | gcuacucgggaggcug | 80 | Yes | Yes |
| | | ox_hap | | | | | | | |
| | | 2 | | | | | | | |
| CHOP_81 | −8.22 | chr6_c | + | 3497783-3497853 | 3497793-3497810 | gcugagaucacaccacug | 81 | Yes | Yes |
| | | ox_hap | | | | | | | |
| | | 2 | | | | | | | |
| CHOP_82 | −5.49 | chr6_c | + | 4650556-4650630 | 4650601-4650620 | ggccgggcaugguggcucac | 82 | Yes | Yes |
| | | ox_hap | | | | | | | |
| | | 2 | | | | | | | |
| CHOP_83 | 177.22 | chr6_c | − | 226217-226295 | 226264-226285 | ggguguagcucagugguagagc | 83 | Yes | Yes |
| | | ox_hap | | | | | | | |
| | | 2 | | | | | | | |
| CHOP_84 | −5.37 | chr6_c | + | 3413207-3413277 | 3413217-3413234 | gucucgaacuccugaccu | 84 | Yes | Yes |
| | | ox_hap | | | | | | | |
| | | 2 | | | | | | | |
| CHOP_85 | −1.27 | chr6_c | − | 1597329-1597407 | 1597339-1597360 | uccugaccucgugauccgcccg | 85 | | Yes |
| | | ox_hap | | | | | | | |
| | | 2 | | | | | | | |
| CHOP_86 | −2.65 | chr6_c | − | 3089988-3090066 | 3089998-3090019 | ugcugggauuacaggugugagc | 86 | Yes | Yes |
| | | ox_hap | | | | | | | |
| | | 2 | | | | | | | |
| CHOP_87 | −2.52 | chr6_c | − | 4297806-4297894 | 4297858-4297884 | uguagucccagcuacucgggaggcuga | 87 | Yes | Yes |
| | | ox_hap | | | | | | | |
| | | 2 | | | | | | | |
| CHOP_88 | −10.98 | chr6_c | + | 4456674-4456756 | 4456684-4456707 | uugauuugcauuucucugauggcc | 88 | | Yes |
| | | ox_hap | | | | | | | |
| | | 2 | | | | | | | |
| CHOP_89 | −12.15 | chr6_c | + | 55320-55390 | 55363-55380 | uuuguauuuuuaguagag | 89 | | Yes |
| | | ox_hap | | | | | | | |
| | | 2 | | | | | | | |

Identified novel miRNA of the MHC derived from the analysis of RNA-Seq. For each miRNA, the identified locus of both the precursor and mature miRNA is provided in addition to the mature miRNA sequence. Functional evidence including whether or not the biogenesis of the mature miRNA was found to be Dicer dependent and whether or not the mature miRNA was found to be loaded onto argonaute (AGO) is also provided.

SUPPLEMENTAL TABLE 2

| Novel miRNA ID | Annotated miRNA ID | Alignment Score |
|---|---|---|
| CHOP_1 | hsa-miR-1254 | 46.3 |
| CHOP_10 | hsa-miR-1273g-3p | 50.7 |
| CHOP_11 | hsa-miR-1469 | 36.3 |
| CHOP_12 | hsa-miR-1285-3p | 49.0 |
| CHOP_13 | hsa-miR-1285-3p | 44.3 |
| CHOP_14 | hsa-miR-323a-5p | 37.3 |
| CHOP_15 | hsa-miR-6850-3p | 38.0 |
| CHOP_16 | hsa-miR-3978 | 37.7 |
| CHOP_17 | hsa-miR-128-1-5p | 35.3 |
| CHOP_18 | hsa-miR-5001-5p | 40.3 |
| CHOP_19 | hsa-miR-4745-3p | 39.3 |
| CHOP_2 | hsa-miR-1273g-3p | 48.3 |
| CHOP_20 | hsa-miR-5006-5p | 35.3 |
| CHOP_21 | hsa-miR-6759-3p | 47.7 |
| CHOP_22 | hsa-miR-3150b-5p | 44.3 |
| CHOP_23 | hsa-miR-6878-3p | 37.0 |
| CHOP_24 | hsa-miR-1909-3p | 35.3 |
| CHOP_24 | hsa-miR-298 | 35.3 |
| CHOP_25 | hsa-miR-544a | 26.3 |
| CHOP_26 | hsa-miR-6849-3p | 38.7 |
| CHOP_27 | hsa-miR-1273h-3p | 52.3 |
| CHOP_28 | hsa-miR-1303 | 40.3 |
| CHOP_29 | hsa-miR-1285-5p | 40.3 |
| CHOP_3 | hsa-miR-7851-3p | 39.7 |
| CHOP_30 | hsa-miR-7974 | 43.7 |
| CHOP_31 | hsa-miR-4746-3p | 40.0 |
| CHOP_32 | hsa-miR-7974 | 39.3 |
| CHOP_33 | hsa-miR-5589-5p | 35.7 |
| CHOP_34 | hsa-miR-584-3p | 41.0 |
| CHOP_35 | hsa-miR-1273a | 46.0 |
| CHOP_36 | hsa-miR-941 | 40.3 |
| CHOP_37 | hsa-miR-6805-3p | 57.7 |
| CHOP_38 | hsa-miR-1273g-3p | 55.3 |
| CHOP_39 | hsa-miR-1285-3p | 44.3 |
| CHOP_4 | hsa-miR-1273g-3p | 51.0 |
| CHOP_40 | hsa-miR-4512 | 48.0 |
| CHOP_41 | hsa-miR-8057 | 29.0 |
| CHOP_42 | hsa-miR-1273g-3p | 59.7 |
| CHOP_43 | hsa-miR-3135a | 39.7 |
| CHOP_44 | hsa-miR-3194-5p | 42.0 |
| CHOP_45 | hsa-miR-1254 | 46.7 |
| CHOP_46 | hsa-miR-4665-3p | 46.3 |
| CHOP_47 | hsa-miR-3620-3p | 50.3 |
| CHOP_48 | hsa-miR-6511b-3p | 44.3 |
| CHOP_49 | hsa-miR-127-5p | 40.0 |
| CHOP_5 | hsa-miR-6781-5p | 34.3 |
| CHOP_50 | hsa-miR-127-5p | 40.0 |
| CHOP_51 | hsa-miR-1908-3p | 41.3 |
| CHOP_52 | hsa-miR-489-5p | 30.3 |
| CHOP_53 | hsa-miR-653-5p | 29.3 |
| CHOP_54 | hsa-miR-3184-3p | 44.7 |
| CHOP_55 | hsa-miR-4695-3p | 47.7 |
| CHOP_56 | hsa-miR-3960 | 45.3 |
| CHOP_57 | hsa-miR-1470 | 40.7 |
| CHOP_58 | hsa-miR-7974 | 38.7 |
| CHOP_59 | hsa-miR-1225-5p | 37.3 |
| CHOP_6 | hsa-miR-6853-5p | 36.0 |
| CHOP_60 | hsa-miR-5087 | 29.0 |
| CHOP_61 | hsa-miR-196b-3p | 36.0 |
| CHOP_62 | hsa-miR-590-3p | 25.7 |
| CHOP_63 | hsa-miR-6727-3p | 44.0 |
| CHOP_64 | hsa-miR-1273h-3p | 52.7 |
| CHOP_65 | hsa-miR-7974 | 38.3 |
| CHOP_66 | hsa-miR-6795-5p | 30.3 |
| CHOP_66 | hsa-miR-8085 | 30.3 |
| CHOP_67 | hsa-miR-6508-3p | 41.0 |
| CHOP_68 | hsa-miR-508-3p | 24.3 |
| CHOP_69 | hsa-miR-3135a | 42.0 |
| CHOP_7 | hsa-miR-3156-5p | 27.0 |
| CHOP_70 | hsa-miR-7974 | 38.3 |
| CHOP_71 | hsa-miR-143 | 39.3 |
| CHOP_72 | hsa-miR-1266-5p | 36.7 |
| CHOP_72 | hsa-miR-1291 | 36.7 |
| CHOP_72 | hsa-miR-3135a | 36.7 |
| CHOP_73 | hsa-miR-1273a | 48.7 |
| CHOP_74 | hsa-miR-3135a | 44.3 |
| CHOP_75 | hsa-miR-6862-3p | 48.7 |
| CHOP_76 | hsa-miR-7974 | 40.3 |
| CHOP_77 | hsa-miR-4515 | 41.7 |
| CHOP_78 | hsa-miR-636 | 36.7 |
| CHOP_79 | hsa-miR-3960 | 43.7 |
| CHOP_8 | hsa-miR-584-3p | 41.3 |
| CHOP_80 | hsa-miR-3130-3p | 34.7 |
| CHOP_81 | hsa-miR-6857-3p | 37.7 |
| CHOP_82 | hsa-miR-1972 | 46.7 |
| CHOP_83 | hsa-miR-5001-5p | 38.7 |
| CHOP_84 | hsa-miR-3184-3p | 37.0 |
| CHOP_84 | hsa-miR-6793-3p | 37.0 |
| CHOP_85 | hsa-miR-1908-3p | 47.7 |
| CHOP_86 | hsa-miR-619-5p | 47.3 |
| CHOP_87 | hsa-miR-5585-5p | 50.7 |
| CHOP_88 | hsa-miR-7107-3p | 37.3 |
| CHOP_89 | hsa-miR-544a | 22.7 |
| CHOP_9 | hsa-miR-1273g-3p | 55.3 |

SUPPLEMENTAL TABLE 3

| | Disease | | |
|---|---|---|---|
| | Associated | | Novel |
| Associated Disease/Trait | SNP | Genomic Context | miRNA ID |
| Age-related hearing impairment | rs6904029 | non coding transcript | CHOP_34 |
| | | exon variant | |
| Age-related macular degeneration | rs12153855 | intron variant | CHOP_11 |
| | | | CHOP_64 |
| | rs2071277 | intron variant | CHOP_46 |
| Antinuclear antibody levels | rs2395185 | intron variant | CHOP_66 |
| Arthritis (juvenile idiopathic) | rs2395148 | intron variant | CHOP_52 |
| Asthma | rs3129943 | intron variant | CHOP_52 |
| Atopic dermatitis | rs12153855 | intron variant | CHOP_11 |
| | | | CHOP_64 |
| Autism spectrum disorder | rs3132581 | intron variant | CHOP_19 |
| | | | CHOP_25 |
| | | | CHOP_42 |
| | | | CHOP_47 |
| | | | CHOP_9 |
| Bipolar disorder and schizophrenia | rs2524005 | upstream gene variant | CHOP_34 |
| | rs886424 | non coding transcript | CHOP_42 |
| | | exon variant | CHOP_47 |
| | | | CHOP_9 |
| Blood metabolite ratios | rs1046080 | missense variant | CHOP_23 |
| | | | CHOP_4 |
| | | | CHOP_61 |
| Cholesterol, total | rs3177928 | 3′ UTR variant | CHOP_43 |
| Complement C3 and C4 levels | rs11575839 | synonymous variant | CHOP_2 |
| | | | CHOP_33 |
| | | | CHOP_39 |
| | rs2071278 | intron variant | CHOP_11 |
| | | | CHOP_27 |
| | | | CHOP_46 |
| | | | CHOP_60 |
| | | | CHOP_64 |
| | | | CHOP_7 |
| Crohns disease | rs1799964 | upstream gene variant | CHOP_32 |
| | rs9258260 | upstream gene variant | CHOP_53 |
| | rs9271366 | intergenic variant | CHOP_26 |
| | | | CHOP_43 |
| | | | CHOP_52 |
| | | | CHOP_66 |
| Cutaneous lupus erythematosus | rs3094067 | intron variant | CHOP_13 |
| | | | CHOP_16 |
| | | | CHOP_34 |
| | | | CHOP_38 |
| | | | CHOP_51 |
| | | | CHOP_8 |
| | rs3094084 | upstream gene variant | CHOP_25 |
| | rs3131060 | downstream gene variant | CHOP_42 |
| | | | CHOP_47 |
| | | | CHOP_9 |
| | rs9267531 | non coding transcript | CHOP_12 |
| | | exon variant | CHOP_14 |
| | | | CHOP_23 |
| | | | CHOP_28 |
| | | | CHOP_31 |
| | | | CHOP_4 |
| | | | CHOP_48 |
| | | | CHOP_56 |
| | | | CHOP_61 |
| | | | CHOP_65 |
| Diastolic blood pressure | rs805303 | intron variant | CHOP_23 |
| | | | CHOP_61 |
| Disc degeneration (lumbar) | rs10046257 | regulatory region variant | CHOP_40 |
| | rs10214886 | intergenic variant | CHOP_40 |
| | rs1029295 | intergenic variant | CHOP_40 |
| | rs1029296 | intergenic variant | CHOP_40 |
| | rs11969002 | upstream gene variant | CHOP_40 |
| | rs3749982 | upstream gene variant | CHOP_40 |
| | rs6457690 | intergenic variant | CHOP_40 |
| | rs6936004 | intergenic variant | CHOP_40 |
| | rs7744666 | upstream gene variant | CHOP_40 |
| | rs9469300 | upstream gene variant | CHOP_40 |
| Drug-induced liver injury (amoxicillin- | rs2523822 | intergenic variant | CHOP_34 |
| clavulanate) | | | |
| Emphysema imaging phenotypes | rs2070600 | missense variant | CHOP_11 |
| | | | CHOP_12 |
| | | | CHOP_14 |
| | | | CHOP_27 |
| | | | CHOP_28 |
| | | | CHOP_46 |
| | | | CHOP_56 |
| | | | CHOP_6 |
| | | | CHOP_60 |
| | | | CHOP_64 |
| | | | CHOP_65 |
| | | | CHOP_7 |
| Epstein Barr virus nuclear antigen 1 | rs2516049 | intron variant | CHOP_66 |
| IgG levels | | | |
| Febrile seizures (MMR vaccine-related) | rs3130618 | missense variant | CHOP_61 |
| Graves disease | rs4313034 | non coding transcript | CHOP_34 |
| | | exon variant | |
| Hematology traits | rs389884 | non coding transcript | CHOP_11 |
| | | exon variant | CHOP_12 |
| | | | CHOP_14 |
| | | | CHOP_27 |
| | | | CHOP_28 |
| | | | CHOP_46 |
| | | | CHOP_56 |
| | | | CHOP_6 |
| | | | CHOP_60 |
| | | | CHOP_64 |
| | | | CHOP_7 |
| Hepatitis B vaccine response | rs9267665 | intron variant | CHOP_12 |
| | | | CHOP_14 |
| | | | CHOP_23 |
| | | | CHOP_28 |
| | | | CHOP_31 |
| | | | CHOP_4 |
| | | | CHOP_48 |
| | | | CHOP_56 |
| | | | CHOP_61 |
| | | | CHOP_65 |
| Hepatitis C induced liver cirrhosis | rs3129860 | intron variant | CHOP_26 |
| | rs3129860 | intron variant | CHOP_43 |
| | rs3129860 | intron variant | CHOP_52 |
| | rs3129860 | intron variant | CHOP_66 |
| HIV-1 control | rs12198173 | intron variant | CHOP_27 |
| | rs12198173 | intron variant | CHOP_60 |
| | rs12198173 | intron variant | CHOP_7 |
| | rs7756521 | intron variant | CHOP_47 |
| | rs9368699 | 5′ UTR variant | CHOP_56 |
| | rs9368699 | 5′ UTR variant | CHOP_6 |
| Hodgkins lymphoma | rs2395185 | intron variant | CHOP_66 |
| Hypertension | rs805303 | intron variant | CHOP_23 |
| | | | CHOP_61 |
| Idiopathic membranous nephropathy | rs3115663 | non coding transcript | CHOP_61 |
| | | exon variant | |
| | rs3129939 | intron variant | CHOP_52 |
| | rs3130618 | missense variant | CHOP_61 |
| | rs3132580 | missense variant | CHOP_19 |
| | | | CHOP_25 |
| | | | CHOP_42 |
| | | | CHOP_47 |
| | | | CHOP_9 |
| | rs3134792 | intergenic variant | CHOP_2 |
| | | | CHOP_39 |
| | rs389884 | non coding transcript | CHOP_11 |
| | | exon variant | CHOP_12 |
| | | | CHOP_14 |
| | | | CHOP_27 |
| | | | CHOP_28 |
| | | | CHOP_46 |
| | | | CHOP_56 |
| | | | CHOP_6 |
| | | | CHOP_60 |
| | | | CHOP_64 |
| | | | CHOP_7 |
| | rs7775397 | missense variant | CHOP_11 |
| | | | CHOP_26 |
| | | | CHOP_27 |
| | | | CHOP_43 |
| | | | CHOP_46 |
| | | | CHOP_52 |
| | | | CHOP_60 |
| | | | CHOP_64 |
| | | | CHOP_66 |
| | | | CHOP_7 |
| IgG glycosylation | rs16871226 | intron variant | CHOP_15 |
| | | | CHOP_40 |
| | | | CHOP_44 |
| | | | CHOP_45 |
| | rs1794265 | upstream gene variant | CHOP_26 |
| | | | CHOP_43 |
| | | | CHOP_52 |
| | | | CHOP_66 |
| | | | CHOP_15 |
| | rs4711279 | 5′ UTR variant | CHOP_12 |
| | | | CHOP_14 |
| | | | CHOP_28 |
| Immunoglobulin A | rs9271366 | intergenic variant | CHOP_26 |
| | | | CHOP_43 |
| | | | CHOP_52 |
| | | | CHOP_66 |
| Interstitial lung disease | rs3132946 | intron variant | CHOP_46 |
| Laryngeal squamous cell carcinoma | rs2857595 | intergenic variant | CHOP_2 |
| | | | CHOP_32 |
| | | | CHOP_33 |
| | | | CHOP_39 |
| Late-onset myasthenia gravis | rs2071591 | intron variant | CHOP_2 |
| | rs2071591 | intron variant | CHOP_33 |
| LDL cholesterol | rs3177928 | 3′ UTR variant | CHOP_43 |
| Lumiracoxib-related liver injury | rs3129900 | intron variant | CHOP_26 |
| | | | CHOP_43 |
| | | | CHOP_52 |
| | | | CHOP_66 |
| Lung adenocarcinoma | rs3117582 | intron variant | CHOP_12 |
| | | | CHOP_14 |
| | | | CHOP_23 |
| | | | CHOP_28 |
| | | | CHOP_31 |
| | | | CHOP_4 |
| | | | CHOP_48 |
| | | | CHOP_56 |
| | | | CHOP_61 |
| | | | CHOP_65 |
| Lung cancer | rs2395185 | intron variant | CHOP_66 |
| | rs3117582 | intron variant | CHOP_12 |
| | | | CHOP_14 |
| | | | CHOP_23 |
| | | | CHOP_28 |
| | | | CHOP_31 |
| | | | CHOP_4 |
| | | | CHOP_48 |
| | | | CHOP_56 |
| | | | CHOP_61 |
| | | | CHOP_65 |
| Lymphoma | rs9268853 | intron variant | CHOP_66 |
| Marginal zone lymphoma | rs2922664 | upstream gene variant | CHOP_2 |
| | | | CHOP_39 |
| Menopause (age at onset) | rs1046089 | missense variant | CHOP_23 |
| | | | CHOP_61 |
| Metabolic syndrome | rs3099844 | downstream gene variant | CHOP_2 |
| | | | CHOP_39 |
| Multiple sclerosis | rs3129889 | downstream gene variant | CHOP_26 |
| | | | CHOP_43 |
| | | | CHOP_52 |
| | | | CHOP_66 |
| | rs3129934 | intron variant | CHOP_26 |
| | | | CHOP_43 |
| | | | CHOP_52 |
| | | | CHOP_66 |
| | rs3135388 | downstream gene variant | CHOP_26 |
| | | | CHOP_43 |
| | | | CHOP_52 |
| | | | CHOP_66 |
| | rs9271366 | intergenic variant | CHOP_26 |
| | | | CHOP_43 |
| | | | CHOP_52 |
| | | | CHOP_66 |
| Myasthenia gravis | rs9270982 | intron variant | CHOP_26 |
| | | | CHOP_43 |
| | | | CHOP_52 |
| | | | CHOP_66 |
| Myositis | rs3130614 | intron variant | CHOP_2 |
| | | | CHOP_39 |
| Neonatal lupus | rs3099844 | downstream gene variant | CHOP_2 |
| | | | CHOP_39 |
| Parental longevity (mothers age | rs1634726 | intron variant | CHOP_19 |
| at death) | | | CHOP_25 |
| | | | CHOP_35 |
| | | | CHOP_42 |
| | | | CHOP_47 |
| | | | CHOP_57 |
| | | | CHOP_9 |
| Percentage gas trapping | rs2070600 | | CHOP_11 |
| | | | CHOP_12 |
| | | | CHOP_14 |
| | | | CHOP_27 |
| | | | CHOP_28 |
| | | | CHOP_46 |
| | | | CHOP_56 |
| | | | CHOP_6 |
| | | | CHOP_60 |
| | | | CHOP_64 |
| | | | CHOP_65 |
| | | | CHOP_7 |
| Phospholipid levels (plasma) | rs3117181 | intron variant | CHOP_11 |
| | | | CHOP_64 |
| Psoriasis | rs3134792 | intergenic variant | CHOP_2 |
| | | | CHOP_39 |
| Pulmonary function | rs2070600 | missense variant | CHOP_11 |
| | | | CHOP_12 |
| | | | CHOP_14 |
| | | | CHOP_27 |
| | | | CHOP_28 |
| | | | CHOP_46 |
| | | | CHOP_56 |
| | | | CHOP_6 |
| | | | CHOP_60 |
| | | | CHOP_64 |
| | | | CHOP_65 |
| | | | CHOP_7 |
| | rs2857595 | intergenic variant | CHOP_2 |
| | | | CHOP_32 |
| | | | CHOP_33 |
| | | | CHOP_39 |
| Response to angiotensin II receptor | rs7772131 | intron variant | CHOP_19 |
| blocker therapy | | | CHOP_25 |
| | | | CHOP_42 |
| | | | CHOP_47 |
| | | | CHOP_9 |
| Response to antipsychotic treatment | rs12526186 | intron variant | CHOP_42 |
| | | | CHOP_9 |
| Rheumatold arthritis | rs12194148 | upstream gene variant | CHOP_66 |
| | rs12525220 | upstream gene variant | CHOP_26 |
| | | | CHOP_43 |
| | | | CHOP_66 |
| | rs2157337 | upstream gene variant | CHOP_66 |
| | rs6910071 | intron variant | CHOP_52 |
| | rs805297 | intron variant | CHOP_23 |
| | | | CHOP_61 |
| Rheumatoid arthritis (ACPA-negative) | rs2596565 | upstream gene variant | CHOP_2 |
| Schizophrenia | rs1046089 | missense variant | CHOP_39 |
| | | | CHOP_23 |
| | rs3131296 | intron variant | CHOP_61 |
| | | | CHOP_11 |
| | | | CHOP_27 |
| | | | CHOP_46 |
| | | | CHOP_52 |
| | | | CHOP_60 |
| | | | CHOP_64 |
| | | intron variant | CHOP_7 |
| Stevens-Johnson syndrome and toxic | rs2734583 | | CHOP_2 |
| epidermal necrolysis (SJS-TEN) | | intron variant | CHOP_39 |
| Systemic lupus erythematosus | rs1150753 | | CHOP_11 |
| | | | CHOP_27 |
| | | | CHOP_46 |
| | | | CHOP_52 |
| | | | CHOP_60 |
| | | | CHOP_64 |
| | | | CHOP_7 |
| | rs1150754 | intron variant | CHOP_27 |
| | | | CHOP_60 |
| | | | CHOP_7 |
| | rs1270942 | non coding transcript | CHOP_11 |
| | | exon variant | CHOP_12 |
| | | | CHOP_14 |
| | | | CHOP_27 |
| | | | CHOP_28 |
| | | | CHOP_46 |
| | | | CHOP_56 |
| | | | CHOP_6 |
| | | | CHOP_60 |
| | | | CHOP_64 |
| | | | CHOP_7 |
| | rs3131379 | intron variant | CHOP_12 |
| | | | CHOP_14 |
| | | | CHOP_23 |
| | | | CHOP_28 |
| | | | CHOP_31 |
| | | | CHOP_4 |
| | | | CHOP_48 |
| | | | CHOP_56 |
| | | | CHOP_6 |
| | | | CHOP_61 |
| | | | CHOP_65 |
| | rs558702 | intron variant | CHOP_12 |
| | | | CHOP_14 |
| | | | CHOP_23 |
| | | | CHOP_28 |
| | | | CHOP_31 |
| | | | CHOP_4 |
| | | | CHOP_48 |
| | | | CHOP_56 |
| | | | CHOP_6 |
| | | | CHOP_61 |
| | | | CHOP_65 |
| | rs9267531 | non coding transcript | CHOP_12 |
| | | exon variant | CHOP_14 |
| | | | CHOP_23 |
| | | | CHOP_28 |
| | | | CHOP_31 |
| | | | CHOP_4 |
| | | | CHOP_48 |
| | | | CHOP_56 |
| | | | CHOP_61 |
| | | | CHOP_65 |
| Systolic blood pressure | rs805303 | intron variant | CHOP_23 |
| | | | CHOP_61 |
| Type 1 diabetes | rs9268645 | intron variant | CHOP_43 |
| Type 1 diabetes and autoimmune | rs1270942 | non coding transcript | CHOP_11 |
| thyroid diseases | | exon variant | CHOP_12 |
| | | | CHOP_14 |
| | | | CHOP_27 |
| | | | CHOP_28 |
| | | | CHOP_46 |
| | | | CHOP_56 |
| | | | CHOP_6 |
| | | | CHOP_60 |
| | | | CHOP_64 |
| | | | CHOP_7 |
| | rs2523989 | missense variant | CHOP_13 |
| | rs2857595 | intergenic variant | CHOP_2 |
| | | | CHOP_32 |
| | | | CHOP_33 |
| | | | CHOP_39 |
| | rs886424 | non coding transcript | CHOP_42 |
| | | exon variant | CHOP_47 |
| | | | CHOP_9 |
| Ulcerative colitis | rs2395185 | intron variant | CHOP_66 |
| | rs9268853 | intron variant | CHOP_66 |
| | rs9271366 | intergenic variant | CHOP_26 |
| | | | CHOP_43 |
| | | | CHOP_52 |
| | | | CHOP_66 |
| Ulcerative colitis or Crohns disease | rs9271366 | intergenic variant | CHOP_26 |
| | | | CHOP_43 |
| | | | CHOP_52 |
| | | | CHOP_66 |
| Visceral fat | rs13196329 | intron variant | CHOP_26 |
| | | | CHOP_43 |
| | | | CHOP_66 |
| Vitiligo | rs3823355 | upstream gene variant | CHOP_34 |
| Waist-to-hip ratio adjutsted for | rs5020946 | downstream gene variant | CHOP_66 |
| body mass index | | | |

Each novel miRNA (89) that is in LD with a disease associated SNP as annotated by GWAS Catalog is reported along with the associated disease, SNP ID and genomic context of each variant (as annotated by GWAS catalog).

SUPPLEMENTAL TABLE 4

| Gene Symbol | Disease Name | Pubmed ID |
|---|---|---|
| ABCF1 | Graves Disease | 21900946 |
| ABCF1 | Lupus Erythematosus, Systemic | 18204098 |
| ATF6B | Lupus Erythematosus, Systemic | 19851445 |
| BAG6 | Adenocarcinoma of lung (disorder) | 19836008 |
| BAG6 | Carcinoma of lung | 24989925 |
| BAG6 | Carcinoma of lung | 25884493 |
| BAG6 | Degenerative polyarthritis | 25231575 |
| BAG6 | Hypertensive disease | 21909115 |
| BAG6 | Malignant neoplasm of lung | 18978787 |
| BAG6 | Malignant neoplasm of lung | 19654303 |
| BAG6 | Malignant neoplasm of lung | 24989925 |
| BAG6 | Malignant neoplasm of lung | 25884493 |
| BAG6 | Non-Small Cell Lung Carcinoma | 25884493 |
| BAG6 | Osteoarthritis of hip | 25231575 |
| BAG6 | Rheumatoid Arthritis | 21844665 |
| BAG6 | Tuberculosis | 24625963 |
| C2 | Age related macular degeneration | 20385819 |
| C2 | Liver carcinoma | 21105107 |
| C2 | Lupus Erythematosus, Systemic | 18204098 |
| C2 | Lupus Erythematosus, Systemic | 24871463 |
| C2 | Multiple Sclerosis | 17660530 |
| DDX39B | Behcet Syndrome | 20622878 |
| DDX39B | Dermatitis, Atopic | 22197932 |
| GABBR1 | Nasopharyngeal carcinoma | 19664746 |
| HLA-DRA | Asthma | 21804548 |
| HLA-DRA | Asthma | 22694930 |
| HLA-DRA | Azoospermia, Nonobstructive | 22541561 |
| HLA-DRA | Classical Hodgkin's Lymphoma | 22286212 |
| HLA-DRA | Diabetes Mellitus, Insulin-Dependent | 19143813 |
| HLA-DRA | Diabetes Mellitus, Insulin-Dependent | 19430480 |
| HLA-DRA | Leukemia, Lymphocytic, Acute, L1 | 21067287 |
| HLA-DRA | Lupus Erythematosus, Systemic | 18204098 |
| HLA-DRA | Multiple Sclerosis | 17660530 |
| HLA-DRA | Multiple Sclerosis | 19525953 |
| HLA-DRA | Multiple Sclerosis | 22190364 |
| HLA-DRA | Multiple Sclerosis | 23472185 |
| HLA-DRA | Papulopustular Rosacea | 25695682 |
| HLA-DRA | Parkinson Disease | 20711177 |
| HLA-DRA | Parkinson Disease | 21791235 |
| HLA-DRA | Parkinson Disease | 24511991 |
| HLA-DRA | Parkinson Disease | 25720714 |
| HLA-DRA | Rosacea | 25695682 |
| HLA-DRA | Sarcoidosis | 23151485 |
| HLA-DRA | Systemic Scleroderma | 21779181 |
| HLA-DRA | Ulcerative Colitis | 18836448 |
| HLA-DRA | Ulcerative Colitis | 19122664 |
| HLA-DRA | Ulcerative Colitis | 20228798 |
| HLA-DRA | Ulcerative Colitis | 22694930 |
| HLA-DRB5 | Multiple Sclerosis | 24733291 |
| MSH5 | Lupus Erythematosus, Systemic | 18204446 |
| MSH5 | Lymphoma, Large-Cell, Follicular | 24598796 |
| MSH5 | Lymphoma, Non-Hodgkin | 24598796 |
| MSH5 | Malignant neoplasm of lung | 18978787 |
| MUC21 | Behcet Syndrome | 20622878 |
| MUC21 | Hypothyroidism | 22493691 |
| MUC21 | Stevens-Johnson Syndrome | 21801394 |
| NFKBIL1 | Behcet Syndrome | 20622878 |
| NOTCH4 | Age related macular degeneration | 22694956 |
| NOTCH4 | Bipolar Disorder | 21987052 |
| NOTCH4 | HIV Infections | 24784026 |
| NOTCH4 | IGA Glomerulonephritis | 20595679 |
| NOTCH4 | Lupus Erythematosus, Systemic | 18204098 |
| NOTCH4 | Lupus Erythematosus, Systemic | 21408207 |
| NOTCH4 | Lupus Erythematosus, Systemic | 23084292 |
| NOTCH4 | Malignant neoplasm of prostate | 23535732 |
| NOTCH4 | Multiple Sclerosis | 17660530 |
| NOTCH4 | Rheumatoid Arthritis | 19143814 |
| NOTCH4 | Rheumatoid Arthritis | 21505073 |
| NOTCH4 | Schizophrenia | 19571808 |
| NOTCH4 | Schizophrenia | 21987052 |
| NOTCH4 | Schizophrenia | 23053058 |
| NOTCH4 | Schizophrenia | 25142293 |
| NOTCH4 | Systemic Scleroderma | 21779181 |
| TNXB | Age related macular degeneration | 22694956 |
| TNXB | Dermatitis, Atopic | 23886662 |
| TNXB | Dermatitis, Atopic | 25574825 |
| TNXB | Hypertensive disease | 25249183 |
| TNXB | Lupus Erythematosus, Systemic | 21408207 |
| TRIM31 | Behcet Syndrome | 20622878 |
| TRIM31 | Cardiomegaly | 21348951 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### XI. References

All patents, patent applications, and publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventor is not entitled to antedate such disclosure by virtue of prior disclosure or for any other reason. All statements as to the date or representation as to the contents of these documents is based on the information available to the applicants and does not constitute any admission as to the correctness of the dates or contents of these documents.

The following references, to the extent that they provide exemplary procedural or other details supplementary to those set forth herein, are specifically incorporated herein by reference.
Allawi et al., RNA 10: 1153-1161, 2004.
Ambros et al., RNA (New York, N.Y.) 9: 277-279, 2003.
Ambros, Cell, 57(1), 1989.
Ameres & Zamore, Nat Rev Mol Cell Biol: 1-14, 2013.
An et al., Nucleic acids research 41: 727-737, 2013.
Arruda et al., Expert Rev. Mol. Diagn, 2:487-496, 2002.
Baek et al., Nature, 455(7209): 64-71, 2008.
Bartel, Cell, 116(2): 281-297, 2004.
Bartel, Cell, 136(2): 215-233, 2009.
Boudreau et al., Neuron 81: 294-305, 2014.
Brennecke & Cohen, Genome Biol, 4(9):228, 2003.
Brenner et al., Nature Biotechnology, 18:630-634, 2000.
Brodersen, Nat Rev Mol Cell Biol, 10(2): 141-48, 2009.
Camacho et al., BMC bioinformatics 10: 421, 2009.
Chai et al., Biochemical and biophysical research communications 471: 123-128, 2016.
Chakravarthy et al., Journal of molecular biology 404: 392-402, 2010.
Chang et al., Nature, 430(7001):785-89, 2004.
Cheloufi et al., Nature 465: 584-589, 2010.
Chi et al., Nat Struct Mol Biol, 19(3): 321-27, 2012.
Chi et al., Nature, 460(7254): 479-86, 2009.
Chu et al., Journal of cellular biochemistry 115: 847-853, 2014.
Cifuentes et al., Science (New York N.Y.) 328: 1694-1698, 2010.
Clark et al., International journal of immunogenetics 42: 413-422, 2015.
Dapprich et al., BMC genomics 17: 486, 2016.
Didiano & Hobert, Nat Struct Mol Biol, 13(9):849-51, 2006.
Easow et al., RNA, 13(8): 1198-204, 2007.
Ebert & Shape, RNA, 16:2043-2050, 2010
Ebert et al., Nat. Methods, 4:721-726, 2007.
Eis et al., Nat. Biotechnol., 19:673-676, 2001.
Erhard et al., Genome research 24: 906-919, 2014.
Fabian et al., Annu Rev Biochen, 79:351-79, 2010.
Farh et al., Nature 518: 337-343, 2015.
Farh et al., Science, 310(5755): 1817-21, 2005.
Fehlmann et al., RNA biology 13: 1084-1088, 2016.
Feng et al., RNA (New York N.Y.) 18: 2083-2092, 2012.
Friedlander et al., Genome biology 15: R57, 2014.
Gillen et al., BMC genomics 17: 338, 2016.
Griffiths-Jones, S., Nucleic Acids Research, 34(90001):D140-D144, 2006.
Guo et al., PloS one 11: e0154955.
Ha et al., Genes Dev, 10(23): p. 3041-50, 1996.
Hafner et al., Cell, 141(1):129-41, 2010.
Horton et al., Immunogenetics 60: 1-18, 2008.
Horton et al., Nature reviews Genetics 5: 889-899, 2004.
International Patent Publication No. WO 2012/106586
Jima et al., Blood 116: e118-127, 2010.
Johnson et al., Bioinformatics (Oxford. England) 24: 2938-2939, 2008.
Johnston & Hobert, Nature, 426(6968): p. 845-49, 2003.
Jonas & Izaurralde, Nature reviews Genetics 16: 421-433, 2015.
Karali et al., Nucleic acids research 44: 1525-1540, 2016.
Kobayashi & Tomari, Biochimica et biophysica acta 1859: 71-81, 2016.
Kodzius et al., Nature Methods, 3: 211-222, 2006
Kozomara & Griffiths-Jones, Nucleic acids research 42: D68-73, 2014.
Ladewig et al., Genome research 22: 1634-1645, 2012.
Lagos-Quintana et al., Science 294, 853-857, 2001.
Lagos-Quintana et al., Current Biology, 12, 735-739, 2002.
Lagos-Quintana et al., RNA, 9, 175-179, 2003.
Lai et al., Mol Cell, 35(5): 610-25, 2009.
Lau et al., Science 294, 858-861, 2001.
Lee & Ambros Science, 294, 862, 2001.
Lee et al., Cell, 75(5): 843-854, 1993.
Liang et al., Molecular medicine reports 15: 1071-1078, 2017.
Lim et al., Science., March 7; 299(5612): 1540, 2003b.
Lim, et al., Genes & Development, 17, p. 991-1008, 2003a.
Llorens et al., BMC Genomics 14: 104, 2013.
Loher et al., Oncotarget 5: 8790-8802, 2014.
Londin et al., Proceedings of the National Academy of Sciences of the United States of America 112: E1106-1115, 2015.
Lorenz et al., Algorithms for molecular biology: AMB 6: 26, 2011.
Lu et al., Molecular cancer 13: 218, 2014.
Ludwig et al., Nucleic acids research 44: 3865-3877, 2016.
Ma et al., Heliyon 2: e00148, 2016.
MacArthur et al., Nucleic acids research 45: D896-d901, 2017.
Meiri et al., Nucleic acids research 38: 6234-6246, 2010.
Metzker et al., Nature Reviews, 11:31-46, 2010.
Miranda et al., Cell 126: 1203-1217, 2006.
Ng et al., Tumour biology: the journal of the International Society for Oncodevelopmental Biology and Medicine 35: 2591-2598, 2014.
Pareek et al., J. Appl Genetics, 52:413-435, 2011.
Perkel BioTechniques, 50:93-95, 2011.
Pillai et al., Breast cancer research and treatment 146: 85-97, 2014.
Pinero et al., Database: the journal of biological databases and curation 2015: bav028, 2015.
Pinero et al., Nucleic acids research 45: D833-d839, 2017.
Ple et al., PloS one 7: e50746, 2012.
Popovic et al., Blood 113: 3314-3322, 2009.
Reinhart, B. J., et al., Nature, 403(6772):901-6, 2000.
Rigoutsos & Tsirigos, in MicroRNAs in Development and Cancer Molecular Medicine and Medicinal Chemistry Ch. 10, ed. F. J. Slack. Vol. 1, 2010: Imperial College Press
Rigoutsos, Cancer Research, 69(8):3245-3248, 2009.
Ruvkun& Giusto, Nature, 338(6213): 313-19, 1989.
Saha et al., Cell, 88:243-251, 1997.
Salem et al., BMC genomics 17: 566, 2016.
Selbach et al., Nature, 455(7209):58-63, 2008.
Shang et al., Oncogene 33: 3267-3276, 2014.
Singh et al., Chem. Soc. Rev., 9, 2054-2070, 2010.
Skalsky et al., PLoS Pathog, 8(1): e1002484, 2012.
Stark et al., Cell, 123(6): 1133-46, 2005
Stewart et al., Genome research 14: 1176-1187, 2004.
Swami, Nature Reviews Genetics, 11:530-531, 2010
Tay et al., Nature, 455(7216): 1124-1128, 2008.
Taylor et al., Nature structural & molecular biology 20: 662-670, 2013.
Thomas, Nat Struct Mol Biol, 17(10): 1169-74, 2010.
Tran et al., RNA (New York, N.Y.) 21: 775-785, 2015.
Tran Vdu et al., RNA (New York, N.Y.) 21: 775-785, 2015.
U.S. Pat. No. 3,270,960
U.S. Pat. No. 3,773,919
U.S. Pat. No. 6,136,540
Urquhart, et al., Ann. Rev. Pharmacol. Toxicol. 24: 199-236 (1984)
Velculescu et al., Science, 270:484-487, 1995.
Vella et al., Genes Dev, 18(2): 132-37, 2004
Voelkerding et al., Clinical Chemistry, 55:641-658, 2009.
Welter et al., Nucleic acids research 42: D1001-1006, 2014.
Wightman et al., Cell, 75(5):855-862, 1993.
Xia et al., Sci Rep, 2:569, 2012
Yang & Lai, Cell cycle (Georgetown, Ill.) 9: 4455-4460, 2010.
Yuan et al., BMC bioinformatics 7: 85, 2006.
Zhang et al., J. Genet Genomics, 38:95-109, 2011.
Zisoulis et al., Nat Struct Mol Biol, 17(2): 173-79, 2010.

What is claimed:

1. A method of designing an miRNA comprising:
(a) providing a target nucleic acid sequence;
(b) generating in silico a population of putative pre-miRNA hairpin sequences having sequences of 58 to about 110 bases;
(c) performing in silico folding of said putative pre-miRNA hairpin sequences to determine secondary structure and Gibbs minimum free energy (MFE);
(d) filtering in silico said putative pre-miRNA hairpin sequences based on linearity of secondary structure and Gibbs MFE of less than about 20 Kcal/mol;
(e) filtering in silico the putative pre-miRNA hairpin sequences remaining after step (d) to remove pre-miRNA hairpin sequences lacking resemblance with annotated putative pre-miRNA hairpin sequences from the genome of the target nucleic acid sequence;
(f) filtering in silico the putative pre-miRNA hairpin sequences remaining after step (e) to remove sequences that overlap annotated exons of protein coding genes from the genome of the target nucleic acid sequence; and
(g) merging overlapping the pre-miRNA hairpin sequences remaining after step (f) into a single locus.

2. The method of claim 1, further comprising synthesizing at least one miRNA sequence from step (g).

3. The method of claim 2, further comprising introducing said at least one miRNA sequence into a cell, and assessing the effect of said at least one miRNA on expression of said target nucleic acid sequence.

4. The method of claim 3, wherein assessing comprises measuring transcript levels for said target nucleic acid sequence, measuring protein levels for the product encoded by said target nucleic acid sequence, measuring the activity of a protein encoded by said target nucleic acid sequence, determining the interaction of said at least one miRNA sequence with an miRNA-interacting molecule (e.g., Argonaute), or assessing the presence, absence or change of a pathologic phenotype.

5. The method of claim 4, wherein said pathologic phenotype is a disease set forth in Table S3 and Table S4.

6. The method of claim 3, wherein said cell is located in vitro.

7. The method of claim 3, wherein said cell is located in vivo.

8. The method of claim 3, wherein said cell is a human cell.

9. The method of claim 3, wherein said cell is a non-human animal cell.

10. The method of claim 1, wherein said target nucleic acid sequence is an MHC sequence.

11. A method of determining whether a subject has, or is at risk of developing, or is at a given stage of a condition afflicting a tissue of interest, comprising measuring in a biological sample from the tissue of interest expression the level of one or more of the miRNAs, wherein the one or miRNAs comprise a sequence selected from SEQ ID NOS: 1-89, wherein the alteration of the level of said one or more miRNAs as compared to the level of the same one or more miRNA in a reference sample is indicative of the subject either having, or being at risk of developing, or is at a given stage of the condition.

12. The method of claim 11, wherein isoforms of the miRNAs are used.

13. The method of claim 11 or 12, wherein the reference sample represents a normal condition of the tissue.

14. The method of claim 11 or 12, wherein the reference sample represents a recognizable stage of an abnormal condition of the tissue.

15. The method of claims 11-14, wherein the expression level of 2, 3, 4, 5, 10, 15, 20, 25, 30, 40, 50, 60, 70, 80 or all 90 miRNAs are measured.

16. A method of identifying a subject having or at risk of developing an immune or inflammatory disorder comprising (a) assessing the expression level of one or more miRNAs selected from SEQ ID NOS: 1-89 in a sample from said subject, and (b) comparing the expression level of said one or more miRNAs in said sample with a normal sample or predetermined control level, wherein an altered expression level of said one or more miRNAs indicates the existence of or increased risk for an immune or inflammatory disorder.

17. The method of claim 16, wherein the miRNA level is elevated.

18. The method of claim 16, wherein the miRNA level is reduced.

19. The method of claim 16, wherein some miRNA levels are elevated, and some are reduced.

20. The method of claims 16-19, wherein the sample is a blood sample.

21. The method of claims 16-19, wherein said inflammatory disorder is cancer.

22. The method of claims 16-19, wherein said immune disorder is an autoimmune disorder.

23. The method of claims 16-19, wherein said immune disorder is IgA nephropathy or IgA deficiency.

24. The method of claims 16-23, wherein said subject is a non-human animal.

25. The method of claims 16-23, wherein said subject is a human.

26. The method of claims 16-25, further comprising treating a subject having or at risk of developing an immune or inflammatory disorder comprising administering to said subject an agonist or antagonist of an miRNA selected from SEQ ID NOS: 1-89.

27. The method of claim 26, wherein said antagonist is a miR antagomir or antisense molecule.

28. The method of claim 26, wherein said agonist/antagonist is formulated in a lipid delivery vehicle.

29. The methods of claim 26, wherein said agonist/antagonist is a nucleic acid containing at least one non-natural base.

30. The method of claim 26, wherein said agonist/antagonist is administered multiple times.

31. The method of claim 30, wherein said agonist/antagonist is administered daily, every other day, every third day, every fourth day, every fifth day, every sixth day, weekly or monthly.

32. The method of claim 26, wherein said agonist/antagonist is administered continuously over a time period exceeding 24 hours.
